(19) United States
(12) Patent Application Publication
(10) Pub. No.: US 2007/0293484 A1
(43) Pub. Date: Dec. 20, 2007

(54) Thiophene Heteroaryl Amines

(75) Inventors: Huiping Guan, Waltham, MA (US); Connie Li Sun, Palo Alto, CA (US); Congxin Liang, Palm Beach Gardens, FL (US); Joanne Johnson, Albany, NY (US); Lisa H. Bourdon, Schenectady, NY (US); Ren Hua Song, Albany, NY (US); Pavel Zhichkin, Albany, NY (US)

Correspondence Address: PFIZER INC, 10555 SCIENCE CENTER DRIVE, SAN DIEGO, CA, 92121, US

(73) Assignee: SUGEN, INC.

(21) Appl. No.: 11/630,588

(22) PCT Filed: May 9, 2005
(86) PCT No.: PCT/IB05/01341

§ 371 Date: Jul. 30, 2007

Related U.S. Application Data
(60) Provisional application No. 60/573,139, filed on May 20, 2004.

(51) Int. Cl. A61K 31/506; A61K 31/5377; A61K 31/541; A61P 17/06; A61P 29/00; A61P 3/10; A61P 35/00; A61P 37/00; A61P 9/00; C07D 409/04; C07D 409/14; C07D 413/14; C07D 417/14

(52) U.S. Cl. 514/227.8; 514/235.8; 514/252.14; 514/275; 544/120; 544/122; 544/295; 544/331; 544/58.2

(57) ABSTRACT

The present invention relates to thiophene heteroaryl amines and their pharmaceutically acceptable salts that modulate the activity of protein kinases and therefore are expected to be useful in the prevention and treatment of protein kinase related cellular disorders such as cancer.

This application claims the benefit of provisional application Ser. No. 60/573,139, filed May 20, 2004, which is incorporated herein by reference in its entirety.

### FIELD OF INVENTION

The present invention relates to thiophene heteroaryl amines that modulate the activity of protein kinases (“PKs”). The compounds of this invention are therefore useful in treating disorders related to abnormal PK activity. Pharmaceutical compositions comprising these compounds, methods of treating diseases utilizing pharmaceutical compositions comprising these compounds and methods of preparing them are also disclosed.

### BACKGROUND

PKs are enzymes that catalyze the phosphorylation of hydroxy groups on tyrosine, serine and threonine residues of proteins. The consequences of this seemingly simple activity are staggering; cell growth, differentiation and proliferation, i.e., virtually all aspects of cell life in one way or another depend on PK activity. Furthermore, abnormal PK activity has been related to a host of disorders, ranging from relatively non-life threatening diseases such as psoriasis to extremely virulent diseases such as glioblastoma (brain cancer).

The PKs can be conveniently broken down into two classes, the protein tyrosine kinases (PTKs) and the serine-threonine kinases (STKs).

One of the prime aspects of PTK activity is their involvement with growth factor receptors. Growth factor receptors are cell-surface proteins. When bound by a growth factor ligand, growth factor receptors are converted to an active form which interacts with proteins on the inner surface of a cell membrane. This leads to phosphorylation on tyrosine residues of the receptor and other proteins and to the formation inside the cell of complexes with a variety of cytoplasm signaling molecules that, in turn, effect numerous cellular responses such as cell division (proliferation), cell differentiation, cell growth, expression of metabolic effects to the extracellular microenvironment, etc. For a more complete discussion, see Schlessinger and Ullrich, Neuron, 9:303-391 (1992) which is incorporated by reference, including any drawings, as if fully set forth herein.

Growth factor receptors with PTK activity are known as receptor tyrosine kinases (“RTKs”). They comprise a large family of transmembrane receptors with diverse biological activity. At present, at least nineteen (19) distinct subfamilies of RTKs have been identified. An example of these is the subfamily designated the “HER” RTKs, which include EGFR (epithelial growth factor receptor), HER2, HER3 and HER4. These RTKs consist of an extracellular glycosylated ligand binding domain, a transmembrane domain and an intracellular cytoplasm catalytic domain that can phosphorylate tyrosine residues on proteins.

Another RTK subfamily consists of insulin receptor (IR), insulin-like growth factor I receptor (IGF-IR) and insulin receptor related receptor (IRR). IR and IGF-1R interact with insulin, IGF-I and IGF-II to form a heterotetramer of two entirely extracellular glycosylated α subunits and two β subunits which cross the cell membrane and which contain the tyrosine kinase domain.

A third RTK subfamily is referred to as the platelet derived growth factor receptor (“PDGFR”) group, which includes PDGFRα, PDGFRβ, CSFIR, c-kit and c-fms. These receptors consist of glycosylated extracellular domains composed of variable numbers of immunoglobin-like loops and an intracellular domain wherein the tyrosine kinase domain is interrupted by unrelated amino acid sequences.

Another group which, because of its similarity to the PDGFR subfamily, is sometimes subsumed into the later group is the fetus liver kinase (“flk”) receptor subfamily. This group is believed to be made up of kinase insert domain-receptor fetal liver kinase-1 (KDR/FLK-1, VEGF-R2), flk-1R, flk-4 and fms-like tyrosine kinase 1 (fit-1).

A further member of the tyrosine kinase growth factor receptor family is the fibroblast growth factor (“FGF”) receptor subgroup. This group consists of four receptors, FGFR1-4, and seven ligands, FGF1-7. While not yet well defined, it appears that the receptors consist of a glycosylated extracellular domain containing a variable number of immunoglobin-like loops and an intracellular domain in which the tyrosine kinase sequence is interrupted by regions of unrelated amino acid sequences.

Still another member of the tyrosine kinase growth factor receptor family is the vascular endothelial growth factor (VEGF″) receptor subgroup. VEGF is a dimeric glycoprotein similar to PDGF but has different biological functions and target cell specificity in vivo. In particular, VEGF is presently thought to play an essential role is vasculogenesis and angiogenesis.

A more complete listing of the known RTK subfamilies is described in Plowman et al., DN&P, 7(6):334-339 (1994) which is incorporated by reference, including any drawings, as if fully set forth herein.

RTKs, CTKs (“non-receptor tyrosine kinases” or “cellular tyrosine kinases”) and STKs (“serine/threonine kinases”) have all been implicated in a host of pathogenic conditions including, significantly, cancer. Other pathogenic conditions which have been associated with PTKs include, without limitation, psoriasis, hepatic cirrhosis, diabetes, angiogenesis, restenosis, ocular diseases, rheumatoid arthritis and other inflammatory disorders, immunological disorders such as autoimmune disease, cardiovascular disease such as atherosclerosis and a variety of renal disorders.

With regard to cancer, two of the major hypotheses advanced to explain the excessive cellular proliferation that drives tumor development relate to functions known to be PK regulated. That is, it has been suggested that malignant cell growth results from a breakdown in the mechanisms that control cell division and/or differentiation. It has been shown that the protein products of a number of proto-oncogenes are involved in the signal transduction pathways that regulate cell growth and differentiation. These protein products of proto-oncogenes include the extracellular growth factors, transmembrane growth factor PTK receptors (RTKs) discussed above, and cytoplasmic PTKs (CTKs) and cytosolic STKs.

### SUMMARY OF THE INVENTION

The present invention is directed to certain aryl-(5-thiophen-3-yl-pyrimidin-2-yl)-amine derivatives which exhibit PK modulating ability and are therefore useful in treating disorders related to abnormal PK activity. In one embodiment the compounds of the invention have activity against one or both of the following receptors PDGFR and FLK.

In one embodiment, the invention provides a compound of the structure: 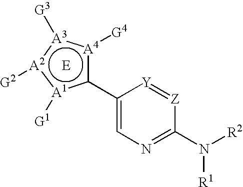
wherein:

one of Y and Z is N and the other is C;

in the ring E, one of A¹, A², A³ and A⁴ is S and the others are C; and

each G¹, G², G³ and G⁴ is independently H or an R⁵ group, except that the one of A¹, A², A³ and A⁴ that is S has no G group attached, and two adjacent G groups can optionally combine to form a 5 or 6 membered aryl, heteroaryl, aliphatic or heteroaliphatic ring;

R¹ is H or C₁₋₆ alkyl;

R² is (i) a six membered aryl or five or six membered heteroaryl, optionally fused with another five or six membered aryl or heteroaryl to form a naphthalene, indene, pentalene or a fused bicyclic heteroaryl, or (ii)-C(═O)NH₂; and wherein each hydrogen in R² independently is optionally substituted by C₁₋₆ alkyl, C₁₋₆ alkenyl, C₁₋₆ akynyl, C₃₋₁₂ cycloalkyl, C₆₋₁₂ aryl, 6 to 12-membered heteroaryl, 3 to 12-membered heteroalicyclic, halogen, hydroxy, C₁₋₆ alkoxy, trihalomethanecarbonyl, sulfonyl, trihalomethanesulfonyl, C-carboxyl, O-carboxyl, C-amido, —OR³, —COR³, —CONR⁸R⁴, —COOR⁸, —NR³R⁴, —CN, —NO₂, —S(O)ₙR³, —S(O₂)NR³R⁴, —NR³R⁴, perfluoro-C₁₋₆ alkyl, —NR³ONR³R⁴, —NR³OR⁴ or —NR³S(O)₂R⁴;

each R³ and R⁴ is independently hydrogen, C₁₋₆ alkyl, C₃₋₁₂ cycloalkyl, C₆₋₁₂ aryl, carbonyl, acetyl, sulfonyl, or trifluoromethanesulfonyl, and R³ and R⁴ are optionally combined to form a 5 or 6-membered heteroalicyclic group;

each R⁵ is independently C₁₋₆ alkyl, C₁₋₆ alkenyl, C₁₋₆ akynyl, C₃₋₁₂ cycloalkyl, C₆₋₁₂ aryl, 6 to 12-membered heteroaryl, 3 to 12-membered heteroalicyclic, halogen, hydroxy, C₁₋₆ alkoxy, trihalomethanecarbonyl, sulfonyl, trihalomethanesulfonyl, C-carboxyl, O-carboxyl, C-amido, —OR³, —COR³, —CONR³R⁴, —COOR³, —NR³R⁴, —CN, —NO₂, —S(O)ₙR³, —S(O₂)NR³R⁴, —NR³R⁴, perfluoro-C₁₋₆ alkyl, —NR³ONR³R⁴, —NR³OR⁴ or —NR³S(O)₂R⁴; and

n is 0, 1 or 2,

or a pharmaceutically acceptable salt, solvate or hydrate thereof.

In a particular aspect of this embodiment, R¹ is H.

In another particular aspect of this embodiment, and in combination with any other particular aspects, R² is C(═O)NH₂, and one of the hydrogen atoms in R² is optionally substituted by C₁₋₆ alkyl, C₁₋₆ alkenyl, C₁₋₆ akynyl, C₃₋₁₂ cycloalkyl, C₆₋₁₂ aryl, 6 to 12-membered heteroaryl, 3 to 12-membered heteroalicyclic, halogen, hydroxy, C₁₋₆ alkoxy, trihalomethanecarbonyl, sulfonyl, trihalomethanesulfonyl, C-carboxyl, O-carboxyl, C-amido, —OR³, —COR³, —CONR³R⁴, —COOR³, —NR³R⁴, —CN, —NO₂, —S(O)ₙR³, —S(O₂)NR³R⁴, —NR³R⁴, perfluoro-C₁₋₆ alkyl, —NR³ONR³R⁴, —NR³OR⁴ or —NR³S(O)₂R⁴.

In another particular aspect of this embodiment, and in combination with any other particular aspects, G¹, G², G³ and G⁴ are H.

In another particular aspect of this embodiment, and in combination with any other particular aspects, R² is a substituted or unsubstituted phenyl.

In another particular aspect of this embodiment, and in combination with any other particular aspects, R² is a phenyl substituted at the 3 or 4 position.

In another particular aspect of this embodiment, and in combination with any other particular aspects, Z is N and Y is C.

In another particular aspect of this embodiment, and in combination with any other particular aspects, Z is C and Y is N.

In another particular aspect of this embodiment, and in combination with any other particular aspects, R² is a five membered heteroaryl selected from the group consisting of thiophene, pyrrole, pyrazole, imidazole, 1,2,3-triazole, 1,2,4-triazole, oxazole, isoxazole, thiazole, isothiazole, 2-sulfonylfuran, 1,2,3-oxadiazole, 1,2,4-oxadiazole, 1,2,5-oxadiazole, 1,3,4-oxadiazole, 1,2,3,4-oxatriazole, 1,2,3,5-oxatriazole, 1,2,3-thiadiazole, 1,2,4-thiadiazole, 1,2,5-thiadiazole, 1,3,4-thiadiazole, 1,2,3,4-thiatriazole, 1,2,3,5-thiatriazole, and tetrazole.

In another particular aspect of this embodiment, and in combination with any other particular aspects, R² is a six membered heteroaryl selected from the group consisting pyridine, pyrazine, pyrimidine, pyridazine, and pyran.

In another particular aspect of this embodiment, and in combination with any other particular aspects, R² is a fused bicyclic heteroaryl selected from the group consisting of benzothiophene, isobenzothiophene, benzofuran, isobenzofuran, chromene, isochromene, indolizine, isoindole, 3H-indole, indole, indazole, purine, 4H-quinolizine, isoquinole, quinole, phthalazine, naphthyridine, quinoxaline, quinazoline, cinnoline, and pteridine.

In another embodiment, the invention provides a compound of the structure: 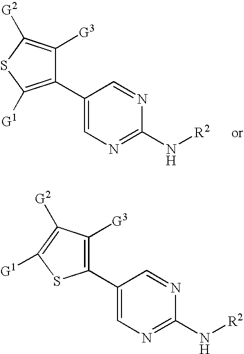
wherein:

each G¹, G² and G³ is independently H or an R⁵ group, and two adjacent G groups can optionally combine to form a 5 or 6 membered aryl, heteroaryl, aliphatic or heteroaliphatic ring;

R² is phenyl or —C(═O)NH₂; and each hydrogen in R² independently is optionally substituted by C₁₋₆ alkyl, C₁₋₆ alkenyl, C₁₋₆ akynyl, C₃₋₁₂ cycloalkyl, C₆₋₁₂ aryl, 6 to 12-membered heteroaryl, 3 to 12-membered heteroalicyclic, halogen, hydroxy, C₁₋₆ alkoxy, trihalomethanecarbonyl, sulfonyl, trihalomethanesulfonyl, C-carboxyl, O-carboxyl, C-amido, —OR³, —COR³, —CONR³R⁴, —COOR³, —NR³R⁴, —CN, —NO₂, —S(O)ₙR³, —S(O₂)NR³R⁴, —NR³R⁴, perfluoro-C₁₋₆ alkyl, —NR³ONR³R⁴, —NR³OR⁴ or —NR³S(O)₂R⁴;

each R³ and R⁴ is independently hydrogen, C₁₋₆ alkyl, C₃₋₁₂ cycloalkyl, C₆₋₁₂ aryl, carbonyl, acetyl, sulfonyl, or trifluoromethanesulfonyl, and R³ and R⁴ are optionally combined to form a 5 or 6-membered heteroalicyclic group;

each R⁵ is independently C₁₋₆ alkyl, C₁₋₆ alkenyl, C₁₋₆ akynyl, C₃₋₁₂ cycloalkyl, C₆₋₁₂ aryl, 6 to 12-membered heteroaryl, 3 to 12-membered heteroalicyclic, halogen, hydroxy, C₁₋₆ alkoxy, trihalomethanecarbonyl, sulfonyl, trihalomethanesulfonyl, C-carboxyl, O-carboxyl, C-amido, —OR³, —COR³, —CONR³R⁴, —COOR³, —NR³R⁴, —CN, —NO₂, —S(O)ₙR³, —S(O₂)NR³R⁴, —NR³R⁴, perfluoro-C₁₋₆ alkyl, —NR³ONR³R⁴, —NR³OR⁴ or —NR³S(O)₂R⁴; and

n is 0, 1 or 2,

or a pharmaceutically acceptable salt, solvate or hydrate thereof.

In a particular aspect of this embodiment, R² is C(═O)NH₂, and one of the hydrogen atoms in R² is optionally substituted by C₁₋₆ alkyl, C₁₋₆ alkenyl, C₁₋₆ akynyl, C₃₋₁₂ cycloalkyl, C₆₋₁₂ aryl, 6 to 12-membered heteroaryl, 3 to 12-membered heteroalicyclic, halogen, hydroxy, C₁₋₆ alkoxy, trihalomethanecarbonyl, sulfonyl, trihalomethanesulfonyl, C-carboxyl, O-carboxyl, C-amido, —OR³, —COR³, —CONR³R⁴, —COOR³, —NR³R⁴, —CN, —NO₂, —S(O)ₙR³, —S(O₂)NR³R⁴, —NR³R⁴, perfluoro-C₁₋₆ alkyl, —NR³ONR³R⁴, —NR³OR⁴ or —NR³S(O)₂R⁴.

In another particular aspect of this embodiment, R² is a substituted or unsubstituted phenyl.

In another embodiment, the invention provides a compound selected from the group consisting of: 4-[(5-thien-3-ylpyrimidin-2-yl)amino]phenol; N-{4-[(5-thien-3-ylpyrimidin-2-yl)amino]phenyl}acetamide; N-(4-morpholin-4-ylphenyl)-5-thien-3-ylpyrimidin-2-amine; 4-amino-N-{4-[(5-thien-3-ylpyrimidin-2-yl)amino]phenyl}benzamide; N-(4-methoxyphenyl)-5-thien-3-ylpyrimidin-2-amine; N-(5-thien-3-ylpyrimidin-2-yl)benzene-1,3-diamine; 3-[(5-thien-3-ylpyrimidin-2-yl)amino]benzoic acid; N-(5-thien-3-ylpyrimidin-2-yl)benzene-1,4-diamine; N-(4-methoxyphenyl)-N′-(5-thien-3-ylpyrimidin-2-yl)urea; N-(4-fluorophenyl)-N′-(5-thien-3-ylpyrimidin-2-yl)urea; 4-[(4-methylpiperazin-1-yl)methyl]-N-{3-[(5-thien-3-ylpyrimidin-2-yl)amino]phenyl}benzamide; 4-[(4-methylpiperazin-1-yl)methyl]-N-{4-[(5-thien-3-ylpyrimidin-2-yl)amino]phenyl}benzamide; N-[4-(5-thien-2-ylpyrimidin-2-ylamino]phenyl]acetamide; N-{3-[(5-thien-2-ylpyrimidin-2-yl)amino]phenyl}acetamide; 4-(5-thiophen-2-yl-pyrimidin-2-ylamino)-phenol; 4-amino-N-{4-[(5-thien-2-ylpyrimidin-2-yl)amino]phenyl}benzamide; 4-[(5-thien-2-ylpyrimidin-2-yl)amino]benzoic acid; N-phenyl-3-[(5-thien-2-ylpyrimidin-2-yl)amino]benzamide; 1-(5-{2-[(3-aminophenyl)amino]pyrimidin-5-yl}thien-2-yl)ethanone; N-[5-(1-benzothien-3-yl)pyrimidin-2-yl]benzene-1,3-diamine; N-(3-(5-(thiophen-3-yl)pyrimidin-2-ylamino)phenyl)-2-(3-((pyrrolidin-1-yl)methyl)pyrrolidin-1-yl)acetamide; 2-[2-(pyrrolidin-1-ylmethyl)pyrrolidin-1-yl]-N-{4-[(5-thien-3-ylpyrimidin-2-yl)amino]phenyl}acetamide; 2-(4-methylpiperazin-1-yl)-N-{4-[(5-thien-3-ylpyrimidin-2-yl)amino]phenyl}acetamide; 2-(4-pyrrolidin-1-ylpiperidin-1-yl)-N-{4-[(5-thien-3-ylpyrimidin-2-yl)amino]phenyl}acetamide; 2-(2-morpholinoethylamino)-N-(4-(5-(thiophen-3-yl)pyrimidin-2-ylamino)phenyl)acetamide; 2-morpholin-4-yl-N-{4-[(5-thien-3-ylpyrimidin-2-yl)amino]phenyl}acetamide; N-(4-(5-(thiophen-3-yl)pyrimidin-2-ylamino)phenyl)-2-(diethylamino)acetamide; 2-(4-hydroxypiperidin-1-yl)-N-{4-[(5-thien-3-ylpyrimidin-2-yl)amino]phenyl}acetamide; 2-pyrrolidin-1-yl-N-{4-[(5-thien-3-ylpyrimidin-2-yl)amino]phenyl}acetamide; 4-pyrrolidin-1-yl-N-{4-[(5-thien-3-ylpyrimidin-2-yl)amino]phenyl}piperidine-1-carboxamide; N-(2-morpholin-4-ylethyl)-N′-{4-[(5-thien-3-ylpyrimidin-2-yl)amino]phenyl}urea; N-[2-(diethylamino)ethyl]-N′-{4-[(5-thien-3-ylpyrimidin-2-yl)amino]phenyl}urea; 3-(pyrrolidin-1-ylmethyl)-N-{4-[(5-thien-3-ylpyrimidin-2-yl)amino]phenyl}pyrrolidine-1-carboxamide; N-{4-[(5-thien-3-ylpyrimidin-2-yl)amino]phenyl}morpholine-4-carboxamide; 4-mothyl-N-{4-[(5-thien-3-ylpyrimidin-2-yl)amino]phenyl}piperazine-1-carboxamide; N-{3-[(5-thien-3-ylpyrimidin-2-yl)amino]phenyl}acetamide; N-isopropyl-N′-(5-thien-3-ylpyrimidin-2-yl)benzene-1,3-diamine; N,N-diethyl-N′-(5-thien-3-ylpyrimidin-2-yl)benzene-1,3-diamine; N-(3-morpholin-4-ylphenyl)-5-thien-3-ylpyrimidin-2-amine; 4-(4-methyl-piperazin-1-ylmethyl)-N-[2-methyl-5-(5-thiophen-3-yl-pyrimidin-2-ylamino)-phenyl]-benzamide; N-(3-(5-(thiophen-3-yl)pyrazin-2-ylamino)phenyl)-2-(diethylamino)acetamide; 2-morpholin-4-yl-N-{3-[(5-thien-3-ylpyrazin-2-yl)amino]phenyl}acetamide; N-{3-[(5-thien-3-ylpyrazin-2-yl)amino]phenyl}morpholine-4-carboxamide; N-(2-morpholin-4-ylethyl)-N′-{3-[(5-thien-3-ylpyrazin-2-yl)amino]phenyl}urea; 4-pyrrolidin-1-yl-N-{3-[(5-thien-3-ylpyrazin-2-yl)amino]phenyl}piperidine-1-carboxamide; N-(3-(5-(thiophen-3-yl)pyrimidin-2-ylamino)phenyl)-2-(3-((pyrrolidin-1-yl)methyl)pyrrolidin-1-yl)acetamide; 4-amino-N-{4-[(5-thien-2-ylpyrimidin-2-yl)amino]phenyl}benzamide; 3-pyrrolidin-1-ylmethyl-pyrrolidine-1-carboxylic acid [3-(5-thien-3-ylpyrimidin-2-yl)amino)-phenyl]-amide; 1-(2-morpholin-4-yl-ethyl)-3-[3-(5-thien-3-ylpyrimidin-2-yl)amino)-phenyl]-urea; 1-(2-diethylamino-ethyl)-3-[3-(5-thien-3-ylpyrimidin-2-yl)amino)-phenyl]-urea; 1-(2-hydroxy-3-morpholin-4-yl-propyl)-3-[3-(5-thiophen-3-yl-pyrimidin-2-ylamino)-phenyl]-urea; 1-[2-(1,1-dioxo-1λ⁸-thiomorpholin-4-yl)ethyl]-3-[3-(5-thiophen-3-yl-pyrimidin-2-ylamino)-phenyl]-urea; 1-[2-(4-methyl-piperazin-1-yl)-ethyl]-3-[3-(5-thiophen-3-yl-pyrimidin-2-ylamino)-phenyl]-urea; 4-pyrrolidin-1-yl-piperidine-1-carboxylic acid [3-(5-thiophen-3-yl-pyrimidin-2-ylamino)-phenyl]-amide; 2-(4-pyrrolidin-1-yl-piperidine-1-yl)-N-[3-(5-thiophen-3-yl-pyrimidin-2-ylamino)-phenyl]-acetamide; 2-(2-morpholin-4-yl-ethylamino)-N-[3-(5-thiophen-3-yl-pyrimidin-2-ylamino)-phenyl]-acetamide; 2-(2-diethylamino-ethylamino)-N-[3-(5-thiophen-3-yl-pyrimidin-2-ylamino)-phenyl]-acetamide; 2-[2-(4-methyl-piperazin-1-yl)-ethylamino]-N-[3-(5-thiophen-3-yl-pyrimidin-2-ylamino)-phenyl]-acetamide; 2-(2-hydroxy-3-morpholin-4-yl-propylamino)-N-[3-(5-thiophen-3-yl-pyrimidin-2-ylamino)-phenyl]-acetamide; 2-[2-(1,1-dioxo-1λ⁶-thiomorpholin-4-yl)-ethylamino]-N-[3-(5-thiophen-3-yl-pyrimidin-2-ylamino)-phenyl]-acetamide; N-[4-(5-thiophen-2-yl-pyrimidin-2-ylamino)-phenyl]-acetamide; 2-morpholin-4-yl-N-[3-(5-thophen-3-yl-pyrimidin-2-ylamino)-phenyl]-acetamide; 2-pyrrolidin-1-yl-N-[3-(5-thiophen-3-yl-pyrimidin-2-ylamino)-phenyl]-acetamide; 5-thiophen-2-yl-pyrimidin-2-ylamine; and 5-thiophen-3-yl-pyrimidin-2-ylamine, or a pharmaceutically acceptable salt, solvate or hydate thereof.

In another embodiment, the invention provides a pharmaceutical composition comprising any of the inventive compounds herein and a pharmaceutically acceptable carrier.

In another embodiment, the invention provides a method for treating FLK-1 or PDGFR mediated disorder in a mammal by administering to the mammal a therapeutically effective amount of a pharmaceutical composition comprising any of the inventive compounds herein and a pharmaceutically acceptable carrier.

In another embodiment, the invention provides a use of any of the inventive compounds herein for the preparation of a medicament useful for treating FLK-1 or PDGFR mediated disorder in a mammal.

In a particular aspect of the method or use, the disorder is a cancer selected from the group consisting of squamous cell carcinoma, astrocytoma, Kaposi's sarcoma, glioblastoma, lung cancer, bladder cancer, head and neck cancer, melanoma, ovarian cancer, prostate cancer, breast cancer, small-cell lung cancer, glioma, colorectal cancer, genitourinary cancer and gastrointestinal cancer.

In another particular aspect of the method or use, the disorder is selected from the group consisting of diabetes, an autoimmune disorder, a hyperproliferation disorder, restenosis, fibrosis, psoriasis, von Heppel-Lindau disease, osteoarthritis, rheumatoid arthritis, angiogenesis, an inflammatory disorder, an immunological disorder and a cardiovascular disorder.

In other embodiments, the invention provides methods of preparing the inventive compounds herein.

In another embodiment, the invention provides methods of identifying a chemical compound that modulates the catalytic activity of a protein kinase by contacting cells expressing the protein kinase with a compound or a salt of the present invention and then monitoring the cells for an effect.

### DETAILED DESCRIPTION

Definitions

Unless otherwise stated the following terms used in the specification and claims have the meanings discussed below:

“Alkyl” refers to a saturated aliphatic hydrocarbon radical including straight chain and branched chain groups of 1 to 20 carbon atoms (whenever a numerical range; e.g. “1-20”, is stated herein, it means that the group, in this case the alkyl group, may contain 1 carbon atom, 2 carbon atoms, 3 carbon atoms, etc. up to and including 20 carbon atoms). More preferably, it is a medium size alkyl having 1 to 10 carbon atoms e.g., methyl, ethyl, propyl, 2-propyl, n-butyl, iso-butyl, tert-butyl, pentyl, and the like. Even more preferably, it is an alkyl of 1 to 6 carbon atoms. Most preferably, it is a lower alkyl having 1 to 4 carbon atoms e.g., methyl, ethyl, propyl, 2-propyl, n-butyl, iso-butyl, or tert-butyl, and the like. Alkyl may be substituted or unsubstituted. When substituted, the substituent group(s) is preferably one or more individually selected from cycloalkyl, aryl, heteroaryl, heteroalicyclic, hydroxy, alkoxy, aryloxy, mercapto, alkylthio, arylthio, cyano, halo, carbonyl, thiocarbonyl, O-carbamyl, N-carbamyl, O-thiocarbamyl, N-thiocarbamyl, C-amido, N-amido, C-carboxy, O-carboxy, nitro, silyl, amino and —NR¹¹R¹², with R¹¹ and R¹² as defined herein.

R¹¹ and R¹² are independently selected from the group consisting of hydrogen, alkyl, cycloalkyl, aryl, carbonyl, acetyl, sulfonyl, trifluoromethanesulfonyl and, combined, a five- or six-member heteroalicyclic ring.

“Cycloalkyl” refers to a 3 to 8 member all-carbon monocyclic ring, an all-carbon 5-member/6-member or 6-member/6-member fused bicyclic ring or a multicyclic fused ring (a “fused” ring system means that each ring in the system shares an adjacent pair of carbon atoms with each other ring in the system) group wherein one or more of the rings may contain one or more double bonds but none of the rings has a completely conjugated pi-electron system. Examples, without limitation, of cycloalkyl groups are cyclopropane, cyclobutane, cyclopentane, cyclopentene, cyclohexane, cyclohexadiene, adamantane, cycloheptane, cycloheptatriene, and the like. A cycloalkyl group may be substituted or unsubstituted. When substituted, the substituent group(s) is preferably one or more individually selected from alkyl, aryl, heteroaryl, heteroalycyclic, hydroxy, alkoxy, aryloxy, mercapto, alkylthio, arylthio, cyano, halo, carbonyl, thiocarbonyl, C-carboxy, O-carboxy, O-carbamyl, N-carbamyl, C-amido, N-amido, nitro, amino and —NR¹¹R¹², with R¹¹ and R¹² as defined above.

“Alkenyl” refers to an alkyl group, as defined herein, consisting of at least two carbon atoms and at least one carbon-carbon double bond. Representative examples include, but are not limited to, ethenyl, 1-propenyl, 2-propenyl, 1-, 2-, or 3-butenyl, and the like.

“Alkynyl” refers to an alkyl group, as defined herein, consisting of at least two carbon atoms and at least one carbon-carbon triple bond. Representative examples include, but are not limited to, ethynyl, 1-propynyl, 2-propynyl, 1-, 2-, or 3-butynyl, and the like.

“Aryl” refers to an all-carbon monocyclic or fused-ring polycyclic (i.e.:, rings which share adjacent pairs of carbon atoms) groups of 1 to 12 carbon atoms having a completely conjugated pi-electron system. Examples, without limitation, of aryl groups are phenyl, naphthalenyl and anthracenyl. The aryl group may be substituted or unsubstituted. When substituted, the substituted group(s) is preferably one or more selected from halo, trihalomethyl, alkyl, hydroxy, alkoxy, aryloxy, mercapto, alkylthio, arylthio, cyano, nitro, carbonyl, thiocarbonyl, C-carboxy, O-carboxy, O-carbamyl, N-carbamyl, O-thiocarbamyl, N-thiocarbamyl, C-amido, N-amido, sulfinyl, sulfonyl, amino and —NR¹¹R¹², with R¹¹ and R¹² as defined herein.

“Heteroaryl” refers to a monocyclic or fused ring (i.e., rings which share an adjacent pair of atoms) group of 5 to 12 ring atoms containing one, two, three or four ring heteroatoms selected from N, O, or S, the remaining ring atoms being C, and, in addition, having a completely conjugated pi-electron system. Examples, without limitation, of unsubstituted heteroaryl groups are pyrrole, furan, thiophene, imidazole, oxazole, thiazole, pyrazole, pyridine, pyrimidine, quinoline, isoquinoline, purine, tetrazole, triazine, and carbazole. The heteroaryl group may be substituted or unsubstituted. When substituted, the substituted group(s) is preferably one or more selected from alkyl, cycloalkyl, halo, trihalomethyl, hydroxy, alkoxy, aryloxy, mercapto, alkylthio, arylthio, cyano, nitro, carbonyl, thiocarbonyl, sulfonamido, C-carboxy, O-carboxy, sulfinyl, sulfonyl, O-carbamyl, N-carbamyl, O-thiocarbamyl, N-thiocarbamyl, C-amido, N-amido, amino and —NR¹¹R¹² with R¹¹ and R¹² as defined above.

A pharmaceutically acceptable heteroaryl is one that is sufficiently stable to be attached to a compound of Formula (I), formulated into a pharmaceutical composition and subsequently administered to a patien in need thereof. Examples, without limitation, of unsubstituted heteroaryl groups are pyrrole, furan, thiophene, imidazole, oxazole, thiazole, pyrazole, pyridine, pyrimidine, quinoline, isoquinoline, purine, tetrazole, triazine, and carbazole as is described about

“Heteroalicyclic” or “heterocycle” refers to a monocyclic or fused ring group having in the ring(s) of 5 to 9 ring atoms in which one or two ring atoms are heteroatoms selected from N, O, or S(O)ₙ (where n is an integer from 0 to 2), the remaining ring atoms being C. The rings may also have one or more double bonds. However, the rings do not have a completely conjugated pi-electron system. Examples, without limitation, of unsubstituted heteroalicyclic groups are pyrrolidino, piperidino, piperazino, morpholino, thiomorpholino, homopiperazino, and the like. The heteroalicyclic ring may be substituted or unsubstituted. When substituted, the substituted group(s) is preferably one or more selected from alkyl, cycloaklyl, halo, trihalomethyl, hydroxy, alkoxy, aryloxy, mercapto, alkylthio, arylthio, cyano, nitro, carbonyl, thiocarbonyl, C-carboxy, O-carboxy, O-carbamyl, N-carbamyl, O-thiocarbamyl, N-thiocarbamyl, sulfinyl, sulfonyl, C-amido, N-amido, amino and —NR¹¹R¹² with R¹¹ and R¹² as defined above. More specifically the term heterocyclyl includes, but is not limited to, tetrahydropyranyl, 2,2-dimethyl-1,3-dioxolane, piperidino, N-methylpiperidin-3-yl, piperazino, N-methylpyrrolidin-3-yl, pyrrolidino, morpholino, thiomorpholino, thiomorpholino-1-oxide, thiomorpholino-1,1-dioxide, 4-ethyloxycarbonylpiperazino, 3-oxopiperazino, 2-imidazolidone, 2-pyrrolidinone, 2-oxohomopiperazino, tetrahydropyrimidin-2-one, and the derivatives thereof. The heterocycle group is optionally substituted with one or two substituents independently selected from halo, lower alkyl, lower alkyl substituted with carboxy, ester hydroxy, or mono or dialkylamino.

“Heterocycloamino” means a saturated cyclic radical of 3 to 8 ring atoms in which at least one of the ring atoms is nitrogen and optionally where one or two additionally ring atoms are heteroatoms selected from N, O, or S(O)ₙ (where n is an integer from 0 to 2), the remaining ring atoms being C, where one or two C atoms may optionally be replaced by a carbonyl group. The heterocycloamino ring may be optionally substituted independently with one, two, or three substituents selected from lower alkyl optionally substituted one or two substituents independently selected from carboxy or ester group, haloalkyl, cyanoalkyl, halo, nitro, cyano, hydroxy, alkoxy, amino, monoalkylamino, dialkylamino, aralkyl, heteroaralkyl, and —COR (where R is alkyl. More specifically the term heterocycloamino includes, but is not limited to, piperidin1-yl, piperazin-1-yl, pyrrolidin-1-yl, morpholin-4-yl, thiomorpholin-4-yl, thiomorpholino-1-oxide, thiomorpholino-1,1-dioxide, 4-ethyloxycarbonylpiperazin-1-yl, 3-oxopiperazin-1-yl, 2-imidazolidon-1-yl, 2-pyrrolidinon-1-yl, 2-oxohomopiperazino, tetrahydropyrimidin-2-one, and the derivatives thereof. Preferably, the heterocycle group is optionally substituted with one or two substituents independently selected from halo, lower alkyl, lower alkyl substituted with carboxy or ester, hydroxy, or mono or dialkylamino. The heterocycloamino group is a subset of the heterocycle group defined above.

“Hydroxy” refers to an —OH group.

“Alkoxy” refers to both an —O-(alkyl) and an —O-(unsubstituted cycloalkyl) group. Representative examples include, but are not limited to, e.g., methoxy, ethoxy, propoxy, butoxy, cyclopropyloxy, cyclobutyloxy, cyclopentyloxy, cyclohexyloxy, and the like.

“Haloalkoxy” refers to both an —O-(haloalkyl) group. Representative examples include, but are not limited to, e.g., trifluoromethoxy, tribromomethoxy, and the like.

“Aryloxy” refers to both an —O-aryl and an —O-heteroaryl group, as defined herein. Representative examples include, but are not limited to, phenoxy, pyridinyloxy, furanyloxy, thienyloxy, pyrimidinyloxy, pyrazinyloxy, and the like, and derivatives thereof.

“Mercapto” refers to an —SH group.

“Alkylthio” refers to both an —S-(alkyl) and an —S-(unsubstituted cycloalkyl) group. Representative examples include, but are not limited to, e.g., methylthio, ethylthio, propylthio, butylthio, cyclopropylthio, cyclobutylthio, cyclopentylthio, cyclohexylthio, and the like.

“Arylthio” refers to both an —S-aryl and an —S-heteroaryl group, as defined herein. Representative examples include, but are not limited to, phenylthio, pyridinylthio, furanylthio, thienylthio, pyrimidinylthio, and the like and derivatives thereof.

“Acyl” or “carbonyl” refers to a —C(O)—R″ group, where R″ is selected from the group consisting of hydrogen, lower alkyl, trihalomethyl, unsubstituted cycloalkyl, aryl optionally substituted with one or more, preferably one, two, or three substituents selected from the group consisting of lower alkyl, trihalomethyl, lower alkoxy, halo and —NR¹¹R¹² groups, heteroaryl (bonded through a ring carbon) optionally substituted with one or more, preferably one, two, or three substitutents selected from the group consisting of lower alkyl, trihaloalkyl, lower alkoxy, halo and —NR¹¹R¹² groups and heteroalicyclic (bonded through a ring carbon) optionally substituted with one or more, preferably one, two, or three substituents selected from the group consisting of lower alkyl, trihaloalkyl, lower alkoxy, halo and —NR¹¹R¹² groups. Representative acyl groups include, but are not limited to, acetyl, trifluoroacetyl, benzoyl, and the like “Aldehyde” refers to an acyl group in which R″ is hydrogen.

“Thioacyl” or “thiocarbonyl” refers to a —C(S)—R″ group, with R″ as defined herein.

A “thiocarbonyl” group refers to a—C(S)—R″ group, with R″ as defined herein.

A “b-carboxy” group refers to a—C(═O)O—R″ group, with R″ as defined herein.

An “O-carboxy” group refers to a—OC(═O)R″ group, with R″ as defined herein.

“Ester” refers to a —C(O)O—R″ group with R″ as defined herein except that R″ cannot be hydrogen.

“Acetyl” group refers to a —C(O)CH₃ group.

“Halo” group refers to fluorine, chlorine, bromine or iodine, preferably fluorine or chlorine.

“Trihalomethyl” group refers to a —CX₃ group wherein X is a halo as defined above.

“Trihalomethanesulfonyl” group refers to a X₃CS(═O)₂— groups with X as defined above.

“Cyano” refers to a —C—N group.

A “sulfinyl” group refers to a—S(═O)—R″ group wherein, in addition to being as defined above, R″ may also be a hydroxy group.

A “sulfonyl” group refers to a—S(═O).sub.2 R″ group wherein, in addition to being as defined above, R″ may also be a hydroxy group.

“S-sulfonamido” refers to a —S(O)₂NR¹¹R¹² group, with R¹¹ and R¹² as defined herein.

“N-sulfonamido” refers to a —NR¹¹S(O)₂R¹² group, with R¹¹ and R¹² as defined herein.

“O-carbamyl” group refers to a —OC(O)NR¹¹R¹² group with R¹¹ and R¹² as defined herein.

“N-carbamyl” refers to an R¹²OC(O)NR¹¹— group, with R¹¹ and R¹² as defined herein.

“O-thiocarbamyl” refers to a —OC(S)NR¹¹R¹² group with R¹¹ and R¹² as defined herein.

“N-thiocarbamyl” refers to a R¹²OC(S)NR¹¹— group, with R¹² and R¹¹ as defined herein.

“Amino” refers to an —R¹¹ and R¹² group, wherein R¹¹ and R¹² are both hydrogen.

“C-amido” refers to a —C(O)NR¹¹R¹² group with R¹¹ and R¹² as defined herein.

“N-amido” refers to a R¹¹C(O)NR¹²— group, with R¹¹ and R¹² as defined herein.

“Nitro” refers to a —NO₂ group.

“Haloalkyl” means an alkyl, preferably lower alkyl as defined above that is substituted with one or more same or different halo atoms, e.g., —CH₂Cl, —CF₃, —CH₂CF₃, —CH₂CCl₃, and the like.

“Hydroxyalkyl” means an alkyl, preferably lower alkyl as defined above that is substituted with one, two, or three hydroxy groups, e.g., hyroxymethyl, 1 or 2-hydroxyethyl, 1,2-, 1,3-, or 2,3-dihydroxypropyl, and the like.

“Aralkyl” means alkyl, preferably lower alkyl as defined above which is substituted with an aryl group as defined above, e.g., —CH₂-phenyl, —(CH₂)₂-phenyl, —(CH₂)₃-phenyl, CH₃CH(CH₃)CH₂-phenyl, and the like and derivatives thereof.

“Heteroaralkyl” group means alkyl, preferably lower alkyl as defined above which is substituted with a heteroaryl group, e.g., —CH₂pyridinyl, —(CH₂)₂pyrimidinyl, —(CH₂)₃imidazolyl, and the like, and derivatives thereof.

“Monoalkylamino” means a radical —NHR where R is an alkyl or unsubstituted cycloalkyl group as defined above, e.g., methylamino, (1-methylethyl)amino, cyclohexylamino, and the like.

“Dialkylamino” means a radical —NRR where each R is independently an alkyl or unsubstituted cycloalkyl group as defined above, e.g., dimethylamino, diethylamino, (1-methylethyl)-ethylamino, cyclohexylmethylamino, cyclopentylmethylamino, and the like.

“Optional” or “optionally” means that the subsequently described event or circumstance may but need not occur, and that the description includes instances where the event or circumstance occurs and instances in which it does not. For example, “heterocycle group optionally substituted with an alkyl group” means that the alkyl may but need not be present, and the description includes situations where the heterocycle group is substituted with an alkyl group and situations where the heterocyclo group is not substituted with the alkyl group.

A “pharmaceutical composition” refers to a mixture of one or more of the compounds described herein, or physiologically/pharmaceutically acceptable salts or prodrugs thereof, with other chemical components, such as physiologically/pharmaceutically acceptable carriers and excipients. The purpose of a pharmaceutical composition is to facilitate administration of a compound to an organism.

A further example of a prodrug might be a short polypeptide, for example, without limitation, a 2-10 amino acid polypeptide, bonded through a terminal amino group to a carboxy group of a compound of this invention wherein the polypeptide is hydrolyzed or metabolized in vivo to release the active molecule. The prodrugs of a compound of Formula (I) are within the scope of this invention.

Additionally, it is contemplated that a compound of Formula (I) would be metabolized by enzymes in the body of the organism such as a human being to generate a metabolite that can modulate the activity of the protein kinases. Such metabolites are within the scope of the present invention.

As used herein, a “physiologically/pharmaceutically acceptable carrier” refers to a carrier or diluent that does not cause significant irritation to an organism and does not abrogate the biological activity and properties of the administered compound.

An “pharmaceutically acceptable excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

As used herein, the term “pharmaceutically acceptable salt” refers to those salts which retain the biological effectiveness and properties of the parent compound. Such salts include:

(i) acid addition salt which is obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methanesulfonic acid, ethanesulfonic acid, p-toluenesulfonic acid, salicylic acid, tartaric acid, citric acid, succinic acid or malonic acid and the like, preferably hydrochloric acid or (L)-malic; or

(2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

“PK” refers to receptor protein tyrosine kinase (RTKs), non-receptor or “cellular” tyrosine kinase (CTKS) and serine-threonine kinases (STKs).

“Method” refers to manners, means, techniques and procedures for accomplishing a given task including, but not limited to, those manners, means, techniques and procedures either known to, or readily developed from known manners, means, techniques and procedures by, practitioners of the chemical, pharmaceutical, biological, biochemical and medical arts.

“Modulation” or “modulating” refers to the alteration of the catalytic activity of RTKs, CTKs and STKs. In particular, modulating refers to the activation of the catalytic activity of RTKs, CTKs and STKs, preferably the activation or inhibition of the catalytic activity of RTKs, CTKs and STKs, depending on the concentration of the compound or salt to which the RTK, CTK or STK is exposed or, more preferably, the inhibition of the catalytic activity of RTKs, CTKs and STKs.

“Catalytic activity” refers to the rate of phosphorylation of tyrosine under the influence, direct or indirect, of RTKs and/or CTKs or the phosphorylation of serine and threonine under the influence, direct or indirect, of STKs.

“Contacting” refers to bringing a compound of this invention and a target PK together in such a manner that the compound can affect the catalytic activity of the PK, either directly, i.e., by interacting with the kinase itself, or indirectly, i.e., by interacting with another molecule on which the catalytic activity of the kinase is dependent. Such “contacting” can be accomplished “in vitro,” i.e., in a test tube, a petri dish or the like. In a test tube, contacting may involve only a compound and a PK of interest or it may involve whole cells. Cells may also be maintained or grown in cell culture dishes and contacted with a compound in that environment. In this context, the ability of a particular compound to affect a PK related disorder, i.e., the IC₅₀ of the compound, defined below, can be determined before use of the compounds in vivo with more complex living organisms is attempted. For cells outside the organism, multiple methods exist, and are well-known to those skilled in the art, to get the PKs in contact with the compounds including, but not limited to, direct cell microinjection and numerous transmembrane carrier techniques.

“In vitro” refers to procedures performed in an artificial environment such as, e.g., without limitation, in a test tube or culture medium.

“In vivo” refers to procedures performed within a living organism such as, without limitation, a mouse, rat or rabbit.

“PK related disorder,” “PK driven disorder,” and “abnormal PK activity” all refer to a condition characterized by inappropriate, i.e., under or, more commonly, over, PK catalytic activity, where the particular PK can be an RTK, a CTK or an STK. Inappropriate catalytic activity can arise as the result of either: (1) PK expression in cells which normally do not express PKs, (2) increased PK expression leading to unwanted cell proliferation, differentiation and/or growth, or, (3) decreased PK expression leading to unwanted reductions in cell proliferation, differentiation and/or growth. Over-activity of a PK refers to either amplification of the gene encoding a particular PK or production of a level of PK activity which can correlate with a cell proliferation, differentiation and/or growth disorder (that is, as the level of the PK increases, the severity of one or more of the symptoms of the cellular disorder increases). Under-activity is, of course, the converse, wherein the severity of one or more symptoms of a cellular disorder increase as the level of the PK activity decreases.

“Treat”, “treating” and “treatment” refer to a method of alleviating or abrogating a PK mediated cellular disorder and/or its attendant symptoms. With regard particularly to cancer, these terms simply mean that the life expectancy of an individual affected with a cancer will be increased or that one or more of the symptoms of the disease will be reduced.

“Organism” refers to any living entity comprised of at least one cell. A living organism can be as simple as, for example, a single eukariotic cell or as complex as a mammal, including a human being.

“Therapeutically effective amount” refers to that amount of the compound being administered which will relieve to some extent one or more of the symptoms of the disorder being treated. In reference to the treatment of cancer, a therapeutically effective amount refers to that amount which has the effect of:
(1) reducing the size of the tumor;
(2) inhibiting (that is, slowing to some extent, preferably stopping) tumor metastasis;
(3) inhibiting to some extent (that is, slowing to some extent, preferably stopping) tumor growth, and/or,
(4) relieving to some extent (or, preferably, eliminating) one or more symptoms associated with the cancer.

“Monitoring” means observing or detecting the effect of contacting a compound with a cell expressing a particular PK. The observed or detected effect can be a change in cell phenotype, in the catalytic activity of a PK or a change in the interaction of a PK with a natural binding partner. Techniques for observing or detecting such effects are well-known in the art.

The above-referenced effect is selected from a change or an absence of change in a cell phenotype, a change or absence of change in the catalytic activity of said protein kinase or a change or absence of change in the interaction of said protein kinase with a natural binding partner in a final aspect of this invention.

“Cell phenotype” refers to the outward appearance of a cell or tissue or the biological function of the cell or tissue. Examples, without limitation, of a cell phenotype are cell size, cell growth, cell proliferation, cell differentiation, cell survival, apoptosis, and nutrient uptake and use. Such phenotypic characteristics are measurable by techniques well-known in the art.

“Natural binding partner” refers to a polypeptide that binds to a particular PK in a cell. Natural binding partners can play a role in propagating a signal in a PK-mediated signal transduction process. A change in the interaction of the natural binding partner with the PK can manifest itself as an increased or decreased concentration of the PK/natural binding partner complex and, as a result, in an observable change in the ability of the PK to mediate signal transduction.

Representative compounds of the present invention are shown in Table 1.

TABLE 1

| Compound No. | Structure | Compound Name(s) |
|---|---|---|
| 1 | 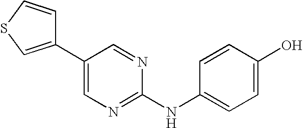 | 4-[(5-thien-3-ylpyrimidin-2- yl)amino]phenol |
| 2 | 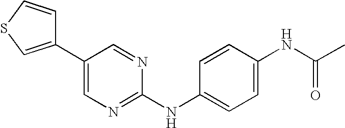 | N-{4-[(5-thien-3-ylpyrimidin-2- yl)amino]phenyl}acetamide |
| 3 | 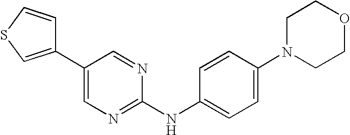 | N-(4-morpholin-4-ylphenyl)-5-thien- 3-ylpyrimidin-2-amine |
| 4 | 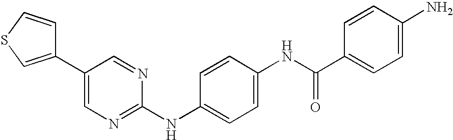 | 4-amino-N-{4-[(5-thien-3-ylpyrimidin- 2-yl)amino]phenyl}benzamide |
| 5 | 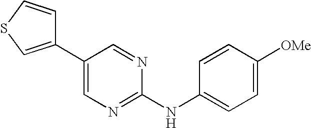 | N-(4-methoxyphenyl)-5-thien-3- ylpyrimidin-2-amine |
| 6 | 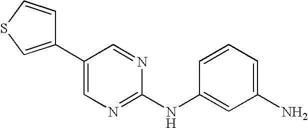 | N-(5-thien-3-ylpyrimidin-2- yl)benzene-1,3-diamine |
| 7 | 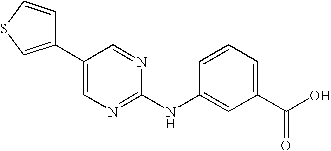 | 3-[(5-thien-3-ylpyrimidin-2- yl)amino]benzoic acid |
| 8 | 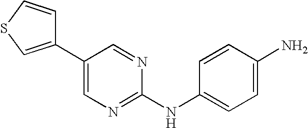 | N-(5-thien-3-ylpyrimidin-2- yl)benzene-1,4-diamine |
| 9 | 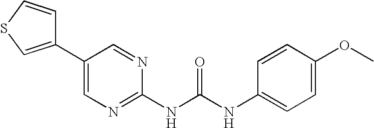 | N-(4-methoxyphenyl)-N′-(5-thien-3- ylpyrimidin-2-yl)urea |
| 10 | 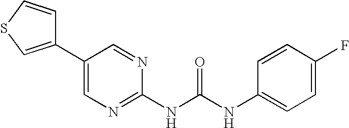 | N-(4-fluorophenyl)-N′-(5-thien-3- ylpyrimidin-2-yl)urea |
| 11 | 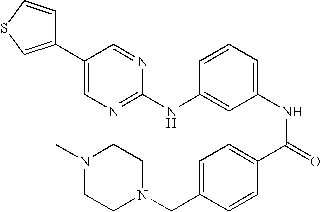 | 4-[(4-methylpiperazin-1-yl)methyl]- N-{3-[(5-thien-3-ylpyrimidin-2- yl)amino]phenyl}benzamide |
| 12 | 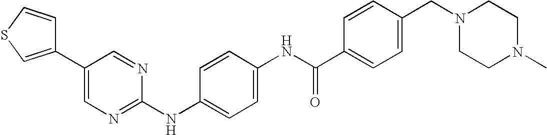 | 4-[(4-methylpiperazin-1-yl)methyl]- N-{4-[(5-thien-3-ylpyrimidin-2- yl)amino]phenyl}benzamide |
| 13 | 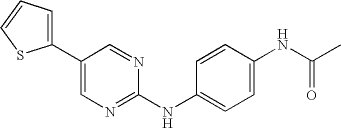 | N-[4-(5-thien-2-ylpyrimidin-2- ylamino]phenyl]acetamide |
| 14 | 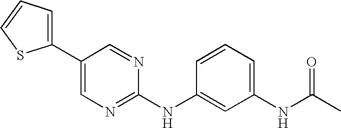 | N-{3-[(5-thien-2-ylpyrimidin-2- yl)amino]phenyl}acetamide |
| 15 | 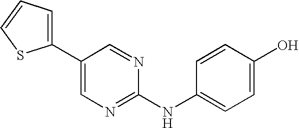 | 4-(5-thiophen-2-yl-pyrimidin-2- ylamino)-phenol |
| 16 | 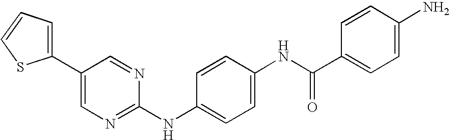 | 4-amino-N-{4-[(5-thien-2-ylpyrimidin- 2-yl)amino]phenyl}benzamide |
| 17 | 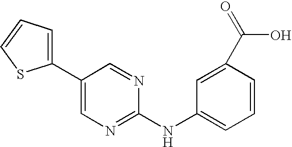 | 3-[(5-thien-2-ylpyrimidin-2- yl)amino]benzoic acid |
| 18 | 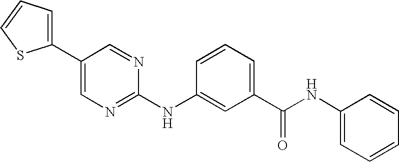 | N-phenyl-3-[(5-thien-2-ylpyrimidin-2- yl)amino]benzamide |
| 19 | 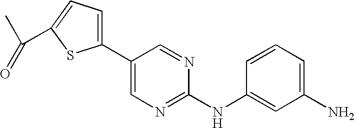 | 1-(5-{2-[(3- aminophenyl)amino]pyrimidin-5- yl}thien-2-yl)ethanone |
| 20 | 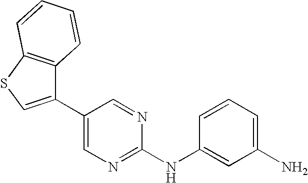 | N-[5-(1-benzothien-3-yl)pyrimidin-2- yl]benzene-1,3-diamine |
| 21 | 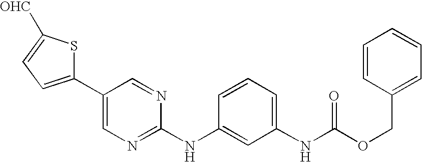 | {3-[5-(5-Formyl-thiophen-2-yl)- pyrimidin-2-ylamino]-phenyl}- carbamic acid benzyl ester |
| 22 | 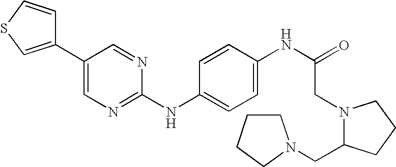 | 2-[2-(pyrrolidin-1-ylmethyl)pyrrolidin- 1-yl]-N-{4-[(5-thien-3-ylpyrimidin-2- yl)amino]phenyl}acetamide |
| 23 | 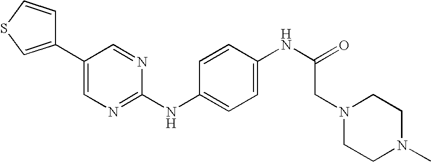 | 2-(4-methylpiperazin-1-yl)-N-{4-[(5- thien-3-ylpyrimidin-2- yl)amino]phenyl}acetamide |
| 24 | 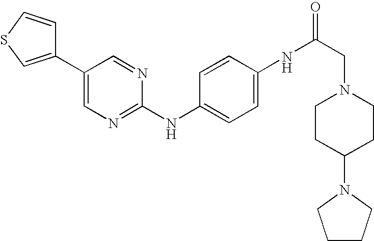 | 2-(4-pyrrolidin-1-ylpiperidin-1-yl)-N- {4-[(5-thien-3-ylpyrimidin-2- yl)amino]phenyl}acetamide |
| 25 | 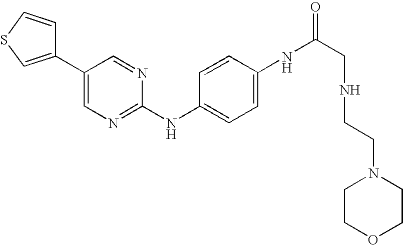 | 2-(2-morpholinoethylamino)-N-(4-(5- (thiophen-3-yl)pyrimidin-2- ylamino)phenyl)acetamide |
| 26 | 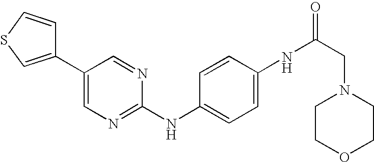 | 2-morpholin-4-yl-N-{4-[(5-thien-3- ylpyrimidin-2- yl)amino]phenyl}acetamide |
| 27 | 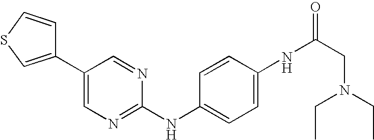 | N-(4-(5-(thiophen-3-yl)pyrimidin-2- ylamino)phenyl)-2- (diethylamino)acetamide |
| 28 | 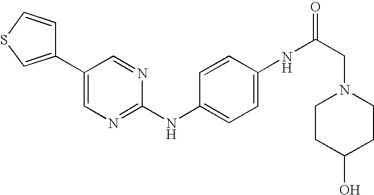 | 2-(4-hydroxypiperidin-1-yl)-N-{4-[(5- thien-3-ylpyrimidin-2- yl)amino]phenyl}acetamide |
| 29 | 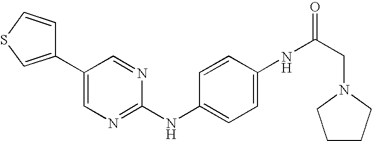 | 2-pyrrolidin-1-yl-N-{4-[(5-thien-3- ylpyrimidin-2- yl)amino]phenyl}acetamide |
| 30 | 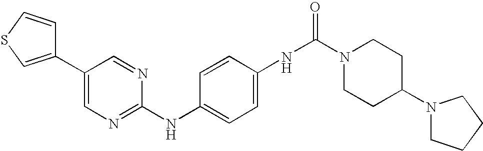 | 4-pyrrolidin-1-yl-N-{4-[(5-thien-3- ylpyrimidin-2- yl)amino]phenyl}piperidine-1- carboxamide |
| 31 | 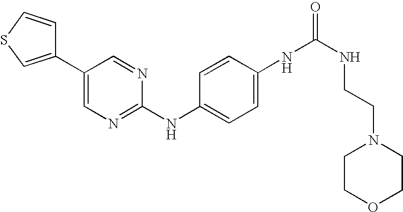 | N-(2-morpholin-4-ylethyl)-N′-{4-[(5- thien-3-ylpyrimidin-2- yl)amino]phenyl}urea |
| 32 | 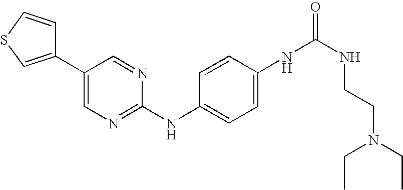 | N-[2-(diethylamino)ethyl]-N′-{4-[(5- thien-3-ylpyrimidin-2- yl)amino]phenyl}urea |
| 33 | 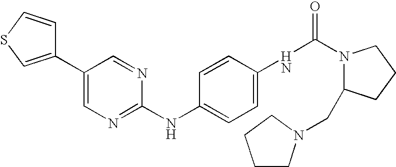 | 2-(pyrrolidin-1-ylmethyl)-N-{4-[(5- thien-3-ylpyrimidin-2- yl)amino]phenyl}pyrrolidine-1- carboxamide |
| 34 | 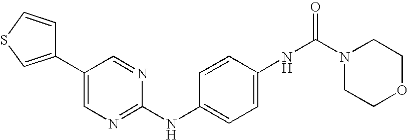 | N-{4-[(5-thien-3-ylpyrimidin-2- yl)amino]phenyl}morpholine-4- carboxamide |
| 35 | 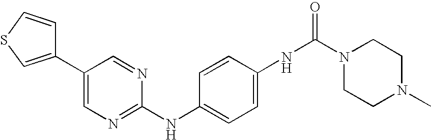 | 4-methyl-N-{4-[(5-thien-3- ylpyrimidin-2- yl)amino]phenyl}piperazine-1- carboxamide |
| 36 | 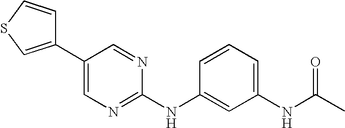 | N-{3-[(5-thien-3-ylpyrimidin-2- yl)amino]phenyl}acetamide |
| 37 | 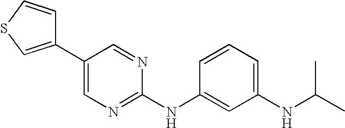 | N-isopropyl-N′-(5-thien-3- ylpyrimidin-2-yl)benzene-1,3- diamine |
| 38 | 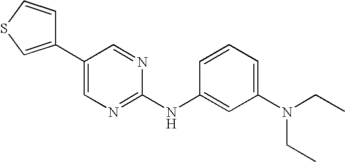 | N,N-diethyl-N′-(5-thien-3-ylpyrimidin- 2-yl)benzene-1,3-diamine |
| 39 | 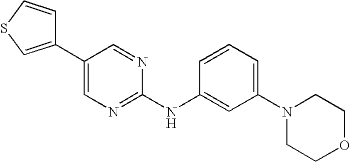 | N-(3-morpholin-4-ylphenyl)-5-thien- 3-ylpyrimidin-2-amine |
| 40 | 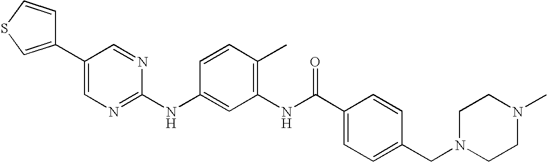 | 4-(4-methyl-piperazin-1-ylmethyl)-N- [2-methyl-5-(5-thiophen-3-yl- pyrimidin-2-ylamino)-phenyl]- benzamide |
| 41 | 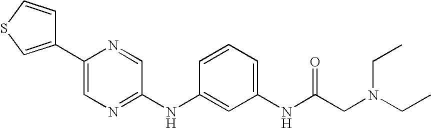 | N-(3-(5-(thiophen-3-yl)pyrazin-2- ylamino)phenyl)-2- (diethylamino)acetamide |
| 42 | 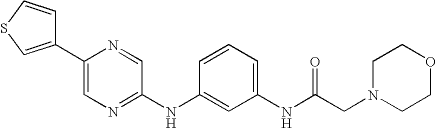 | 2-morpholin-4-yl-N-{3-[(5-thien-3- ylpyrazin-2- yl)amino]phenyl}acetamide |
| 43 | 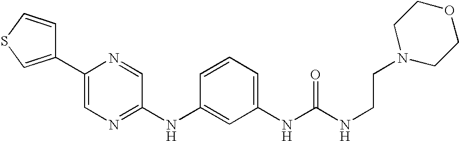 | 1-(3-(5-(thiophen-3-yl)pyrazin-2- ylamino)phenyl)-3-(2- morpholinoethyl)urea |
| 44 | 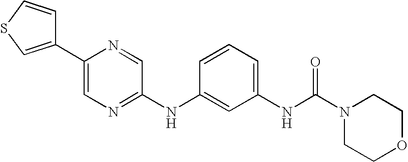 | N-{3-[(5-thien-3-ylpyrazin-2- yl)amino]phenyl}morpholine-4- carboxamide |
| 45 | 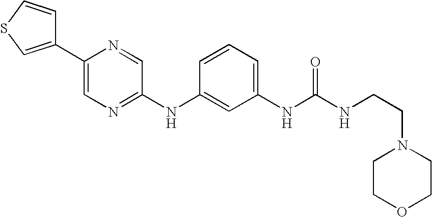 | N-(2-morpholin-4-ylethyl)-N′-{3-[(5- thien-3-ylpyrazin-2- yl)amino]phenyl}urea |
| 46 | 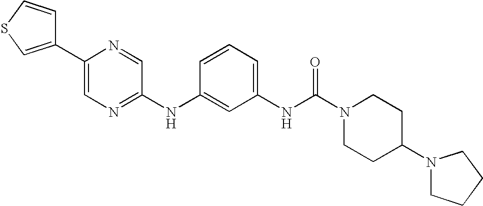 | 4-pyrrolidin-1-yl-N-{3-[(5-thien-3- ylpyrazin-2- yl)amino]phenyl}piperidine-1- carboxamide |
| 47 | 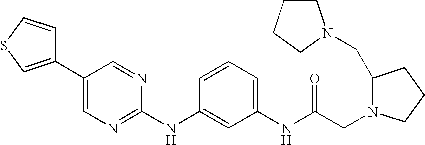 | N-(3-(5-(thiophen-3-yl)pyrimidin-2- ylamino)phenyl)-2-(2-((pyrrolidin-1- yl)methyl)pyrrolidin-1-yl)acetamide |
| 48 | 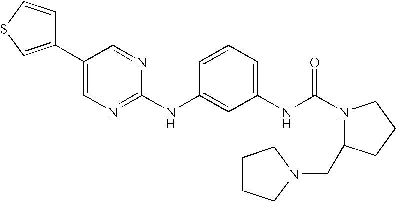 | 2-pyrrolidin-1-ylmethyl-pyrrolidine-1- carboxylic acid [3-(5-thien-3- ylpyrimidin-2-yl)amino)-phenyl]- amide |
| 49 | 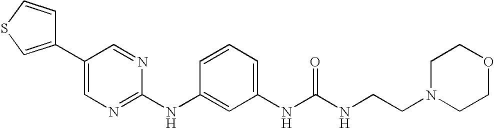 | 1-(2-morpholin-4-yl-ethyl)-3-[3-(5- thien-3-ylpyrimidin-2-yl)amino)- phenyl]-urea |
| 50 | 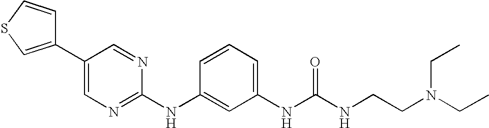 | 1-(2-diethylamino-ethyl)-3-[3-(5- thien-3-ylpyrimidin-2-yl)amino)- phenyl]-urea |
| 51 | 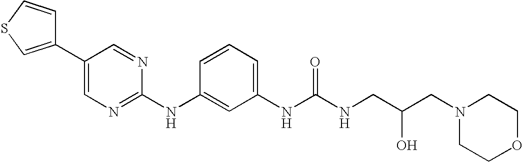 | 1-(2-hydroxy-3-morpholin-4-yl- propyl)-3-[3-(5-thiophen-3-yl- pyrimidin-2-ylamino)-phenyl]-urea |
| 52 | 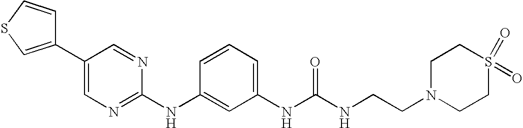 | 1-[2-(1,1-dioxo-1λ⁶-thiomorpholin-4- yl)ethyl]-3-[3-(5-thiophen-3-yl- pyrimidin-2-ylamino)-phenyl]-urea |
| 53 | 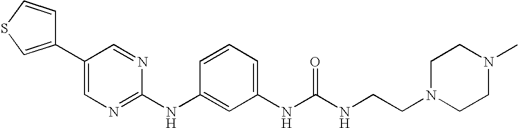 | 1-[2-(4-methyl-piperazin-1-yl)-ethyl]- 3-[3-(5-thiophen-3-yl-pyrimidin-2- ylamino)-phenyl]-urea |
| 54 | 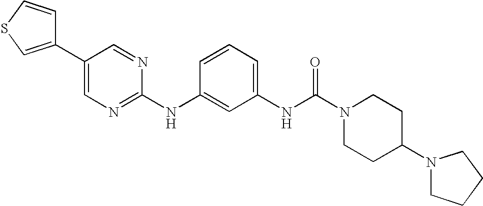 | 4-pyrrolidin-1-yl-piperidine-1- carboxylic acid [3-(5-thiophen-3-yl- pyrimidin-2-ylamino)-phenyl]-amide |
| 55 | 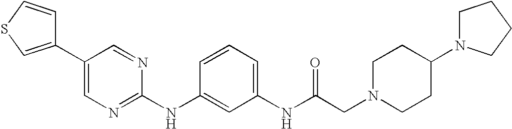 | 2-(4-pyrrolidin-1-yl-piperidine-1-yl)- N-[3-(5-thiophen-3-yl-pyrimidin-2- ylamino)-phenyl]-acetamide |
| 56 | 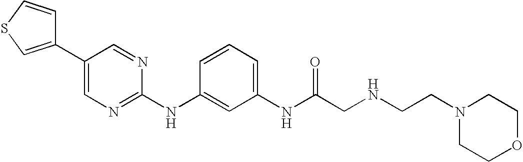 | 2-(2-morpholin-4-yl-ethylamino)-N- [3-(5-thiophen-3-yl-pyrimidin-2- ylamino)-phenyl]-acetamide |
| 57 | 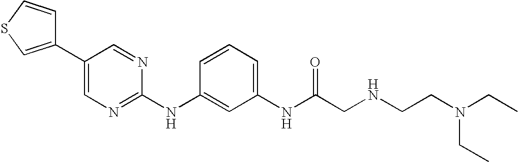 | 2-(2-diethylamino-ethylamino)-N-[3- (5-thiophen-3-yl-pyrimidin-2- ylamino)-phenyl]-acetamide |
| 58 | 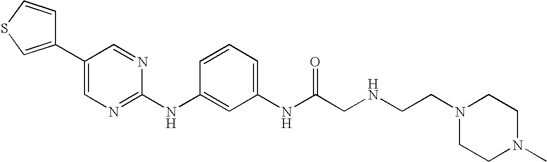 | 2-[2-(4-methyl-piperazin-1-yl)- ethylamino]-N-[3-(5-thiophen-3-yl- pyrimidin-2-ylamino)-phenyl]- acetamide |
| 59 | 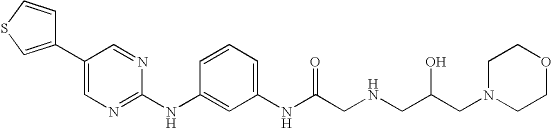 | 2-(2-hydroxy-3-morpholin-4-yl- propylamino)-N-[3-(5-thiophen-3-yl- pyrimidin-2-ylamino)-phenyl]- acetamide |
| 60 | 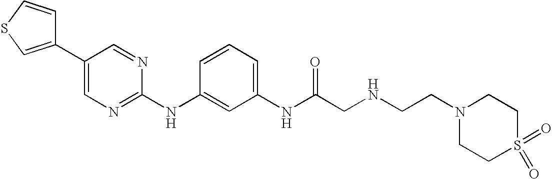 | 2-[2-(1,1-dioxo-1λ⁶-thiomorpholin-4- yl)-ethylamino]-N-[3-(5-thiophen-3- yl-pyrimidin-2-ylamino)-phenyl]- acetamide |
| 61 | 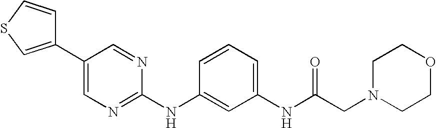 | 2-morpholin-4-yl-N-[3-(5-thiophen-3- yl-pyrimidin-2-ylamino)-phenyl]- acetamide |
| 62 | 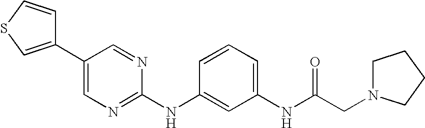 | 2-pyrrolidin-1-yl-N-[3-(5-thiophen-3- yl-pyrimidin-2-ylamino)-phenyl]- acetamide |

Preferred compounds of the present invention display acitivity against a variety of proteins kinases. In particular, preferred compounds display activity against PDGFR and/or FLK-1. The PKs whose catalytic activity is modulated by the compounds of this invention include protein tyrosine kinases of which there are three types, receptor tyrosine kinases (RTKs) and cellular tyrosine kinases (CTKs), and serine-threonine kinases (STKs). RTK mediated signal transduction is initiated by extracellular interaction with a specific growth factor (ligand), followed by receptor dimerization, transient stimulation of the intrinsic protein tyrosine kinase activity and phosphorylation. Binding sites are thereby created for intracellular signal transduction molecules and lead to the formation of complexes with a spectrum of cytoplasmic signaling molecules that facilitate the appropriate cellular response (e.g., cell division, metabolic effects on the extracellular microenvironment, etc.). See, Schlessinger and Ullrich, 1992, Neuron 9:303-391.

Activity of particular compounds of the invention was determined as described in the Examples herein, and is shown in Table 2.

TABLE 2

| | |
|---|---|
| Example | IC₅₀ (μM) |

| No. | FLK-1 | PDGF-R |
|---|---|---|
| 1 | 4.50 | 0.52 |
| 2 | 3.74 | 0.07 |
| 3 | >20 | 0.15 |
| 4 | >20 | 0.50 |
| 5 | >20 | 0.55 |
| 6 | 1.53 | 0.06 |
| 7 | 16.19 | 7.39 |
| 8 | 4.14 | 0.55 |
| 9 | >20 | >20 |
| 10 | >20 | >20 |
| 11 | 0.73 | 0.22 |
| 12 | 2.52 | 0.26 |
| 13 | 9.86 | 0.44 |
| 14 | 4.17 | 0.94 |
| 15 | 4.61 | 1.70 |
| 16 | >20 | 0.99 |
| 17 | >20 | 15.21 |
| 18 | >20 | >20 |
| 19 | 8.60 | 2.40 |
| 20 | >20 | >20 |
| 21 | | |
| 22 | 4.50 | 0.41 |
| 23 | 5.10 | 0.55 |
| 24 | | |
| 25 | 5.40 | 0.47 |
| 26 | 5.60 | 1.10 |
| 27 | 6.00 | 0.55 |
| 28 | 4.90 | 0.57 |
| 29 | 4.00 | 0.43 |
| 30 | 6.20 | 0.50 |
| 31 | 5.20 | 0.78 |
| 32 | 5.70 | 0.53 |
| 33 | 4.55 | 0.66 |
| 34 | 4.70 | 1.00 |
| 35 | 5.40 | 0.47 |
| 36 | 3.50 | 0.66 |
| 37 | 3.70 | 0.56 |
| 38 | 3.00 | 1.80 |
| 39 | 2.00 | 0.15 |
| 40 | 10.00 | 2.60 |
| 41 | >20 | >20 |
| 42 | >20 | >20 |
| 43 | | |
| 44 | | |
| 45 | | |
| 46 | | |
| 47 | 2.30 | 0.18 |
| 48 | 3.50 | 0.21 |
| 49 | 1.90 | 0.14 |
| 50 | 1.70 | 0.16 |
| 51 | 2.00 | 0.12 |
| 52 | 1.60 | 0.36 |
| 53 | 2.30 | 0.22 |
| 54 | 2.30 | 0.18 |
| 55 | 2.70 | 0.19 |
| 56 | 2.40 | 0.09 |
| 57 | 3.10 | 0.23 |
| 58 | 2.50 | 0.08 |
| 58 | 4.00 | 0.43 |
| 59 | 2.70 | 0.09 |
| 60 | 2.40 | 0.06 |
| 61 | 2.50 | 0.94 |
| 62 | 2.80 | 0.20 |

It has been that tyrosine phosphorylation sites on growth factor receptors function as high-affinity binding sites for SH2 (src homology) domains of signaling molecules. Fantl et al., 1992, Cell 69:413-423, Songyang et al., 1994, Mol. Cell. Biol. 14:2777-2785), Songyang et al., 1993, Cell 72:767-778, and Koch et al., 1991, Science 252:668-678. Several intracellular substrate proteins that associate with RIKs have been identified. They may be divided into two principal groups: (1) substrates that have a catalytic domain, and (2) substrates which lack such domain but which serve as adapters and associate with catalytically active molecules. Songyang et al., 1993, Cell 72:767-778. The specificity of the interactions between receptors and SH2 domains of their substrates is determined by the amino acid residues immediately surrounding the phosphorylated tyrosine residue. Differences in the binding affinities between SH2 domains and the amino acid sequences surrounding the phosphotyrosine residues on particular receptors are consistent with the observed differences in their substrate phosphorylation profiles. Songyang et al., 1993, Cell 72:767-778. These observations suggest that the function of each RTK is determined not only by its pattern of expression and ligand availability but also by the array of downstream signal transduction pathways that are activated by a particular receptor. Thus, phosphorylation provides an important regulatory step which determines the selectivity of signaling pathways recruited by specific growth factor receptors, as well as differentiation factor receptors.

STKs, being primarily cytosolic, affect the internal biochemistry of the cell, often as a down-line response to a PTK event. STKs have been implicated in the signaling process which initiates DNA synthesis and subsequent mitosis leading to cell proliferation.

Thus, PK signal transduction results in, among other responses, cell proliferation, differentiation, growth and metabolism. Abnormal cell proliferation may result in a wide array of disorders and diseases, including the development of neoplasia such as carcinoma, sarcoma, glioblastoma and hemangioma, disorders such as leukemia, psoriasis, arteriosclerosis, arthritis and diabetic retinopathy and other disorders related to uncontrolled angiogenesis and/or vasculogenesis.

A precise understanding of the mechanism by which the compounds of this invention inhibit PKs is not required in order to practice the present invention. However, while not hereby being bound to any particular mechanism or theory, it is believed that the compounds interact with the amino acids in the catalytic region of PKs. PKs typically possess a bi-lobate structure wherein ATP appears to bind in the cleft between the two lobes in a region where the amino acids are conserved among PKs. Inhibitors of PKs are believed to bind by non-covalent interactions such as hydrogen bonding, van der Waals forces and ionic interactions in the same general region where the aforesaid ATP binds to the PKs. The compounds disclosed herein thus have utility in in vitro assays for such proteins as well as exhibiting in vivo therapeutic effects through interaction with such proteins.

Additionally, the compounds of the present invention provide a therapeutic approach to the treatment of many kinds of solid tumors, including but not limited to carcinomas, sarcomas including Kaposi's sarcoma, erythroblastoma, glioblastoma, meningioma, astrocytoma, melanoma and myoblastoma. Treatment or prevention of non-solid tumor cancers such as leukemia are also contemplated by this invention. Indications may include, but are not limited to brain cancers, bladder cancers, ovarian cancers, gastric cancers, pancreas cancers, colon cancers, blood cancers, lung cancers and bone cancers.

Further examples, without limitation, of the types of disorders related to inappropriate PK activity that the compounds described herein may be useful in preventing, treating and studying, are cell proliferative disorders, fibrotic disorders and metabolic disorders.

Cell proliferative disorders, which may be prevented, treated or further studied by the present invention include cancer, blood vessel proliferative disorders and mesangial cell proliferative disorders.

Blood vessel proliferative disorders refer to disorders related to abnormal vasculogenesis (blood vessel formation) and angiogenesis (spreading of blood vessels). While vasculogenesis and angiogenesis play important roles in a variety of normal physiological processes such as embryonic development, corpus luteum formation, wound healing and organ regeneration, they also play a pivotal role in cancer development where they result in the formation of new capillaries needed to keep a tumor alive. Other examples of blood vessel proliferation disorders include arthritis, where new capillary blood vessels invade the joint and destroy cartilage, and ocular diseases, like diabetic retinopathy, where new capillaries in the retina invade the vitreous, bleed and cause blindness.

Two structurally related RTKs have been identified to bind VEGF with high affinity: the fms-like tyrosine 1 (flt-I) receptor (Shibuya et al., 1990, Oncogene, 5:519-524; De Vries et al., 1992, Science, 255:989-991) and the KDR/FLK-1 receptor, also known as VEGF-R2. Vascular endothelial growth factor (VEGF) has been reported to be an endothelial cell specific mitogen with in vitro endothelial cell growth promoting activity. Ferrara & Henzel, 1989, Biochein. Biophys. Res. Comm., 161:851-858; Vaisman et al., 1990, J. Biol. Chem., 265:19461-19566. Information set forth in U.S. application Ser. Nos. 08/193,829, 08/038,596 and 07/975,750, strongly suggest that VEGF is not only responsible for endothelial cell proliferation, but also is the prime regulator of normal and pathological angiogenesis. See generally, Klagsburn & Soker, 1993, Current Biology, 3(10)699-702; Houck, et al., 1992, J. Biol. Chem., 267:26031-26037.

Normal vasculogenesis and angiogenesis play important roles in a variety of physiological processes such as embryonic development, wound healing, organ regeneration and female reproductive processes such as follicle development in the corpus luteum during ovulation and placental growth after pregnancy. Folkman & Shing, 1992, J. Biological Chem., 267(16):10931-34. Uncontrolled vasculogenesis and/or angiogenesis has been associated with diseases such as diabetes as well as with malignant solid tumors that rely on vascularization for growth. Klagsburn & Soker, 1993, Current Biology, 3(10):699-702; Folkham, 1991, J. Natl. Cancer Inst., 82:4-6; Weidner, et al., 1991, New Engl. J. Med., 324:1-5.

The surmised role of VEGF in endothelial cell proliferation and migration during angiogenesis and vasculogenesis indicates an important role for the KDR/FLK-1 receptor in these processes. Diseases such as diabetes mellitus (Folkman, 198, in XI^{th} Congress of Thrombosis and Haemostasis (Verstraeta, et al., eds.), pp. 583-596, Leuven University Press, Leuven) and arthritis, as well as malignant tumor growth may result from uncontrolled angiogenesis. See e.g., Folkman, 1971, N. Engl. J. Med., 285:1182-1186. The receptors to which VEGF specifically binds are an important and powerful therapeutic target for the regulation and modulation of vasculogenesis and/or angiogenesis and a variety of severe diseases which involve abnormal cellular growth caused by such processes. Plowman, et al., 1994, DN&P, 7(6):334-339. More particularly, the KDR/FLK-1 receptor's highly specific role in neovascularization make it a choice target for therapeutic approaches to the treatment of cancer and other diseases which involve the uncontrolled formation of blood vessels.

Thus, the present invention provides compounds capable of regulating and/or modulating tyrosine kinase signal transduction including KDR/FLK-1 receptor signal transduction in order to inhibit or promote angiogenesis and/or vasculogenesis, that is, compounds that inhibit, prevent, or interfere with the signal transduced by KDR/FLK-1 when activated by ligands such as VEGF. Although it is believed that the compounds of the present invention act on a receptor or other component along the tyrosine kinase signal transduction pathway, they may also act directly on the tumor cells that result from uncontrolled angiogenesis.

Although the nomenclature of the human and murine counterparts of the generic “flk-I” receptor differ, they are, in many respects, interchangeable. The murine receptor, Flk-1, and its human counterpart, KDR, share a sequence homology of 93.4% within the intracellular domain. Likewise, murine FLK-I binds human VEGF with the same affinity as mouse VEGF, and accordingly, is activated by the ligand derived from either species. Millauer et al., 1993, Cell, 72:835-846; Quinn et al., 1993, Proc. Natl. Acad. Sci. USA, 90:7533-7537. FLK-1 also associates with and subsequently tyrosine phosphorylates human RTK substrates (e.g., PLC-γ or p85) when co-expressed in 293 cells (human embryonal kidney fibroblasts).

Models which rely upon the FLK-1 receptor therefore are directly applicable to understanding the KDR receptor. For example, use of the murine FLK-1 receptor in methods which identify compounds that regulate the murine signal transduction pathway are directly applicable to the identification of compounds which may be used to regulate the human signal transduction pathway, that is, which regulate activity related to the KDR receptor. Thus, chemical compounds identified as inhibitors of KDR/FLK-1 in vitro, can be confirmed in suitable in vivo models. Both in vivo mouse and rat animal models have been demonstrated to be of excellent value for the examination of the clinical potential of agents acting on the KDR/FLK-1 induced signal transduction pathway.

Thus, the present invention provides compounds that regulate, modulate and/or inhibit vasculogenesis and/or angiogenesis by affecting the enzymatic activity of the KDR/FLK-1 receptor and interfering with the signal transduced by KDR/FLK-1. Thus the present invention provides a therapeutic approach to the treatment of many kinds of solid tumors including, but not limited to, glioblastoma, melanoma and Kaposi's sarcoma, and ovarian, lung, mammary, prostate, pancreatic, colon and epidermoid carcinoma. In addition, data suggests the administration of compounds which inhibit the KDR/Flk-1 mediated signal transduction pathway may also be used in the treatment of hemangioma, restenois and diabetic retinopathy.

Furthermore, this invention relates to the inhibition of vasculogenesis and angiogenesis by other receptor-mediated pathways, including the pathway comprising the VEGF receptor.

Receptor tyrosine kinase mediated signal transduction is initiated by extracellular interaction with a specific growth factor (ligand), followed by receptor dimerization, transient stimulation of the intrinsic protein tyrosine kinase activity and autophosphorylation. Binding sites are thereby created for intracellular signal transduction molecules which leads to the formation of complexes with a spectrum of cytoplasmic signalling molecules that facilitate the appropriate cellular response, e.g., cell division and metabolic effects to the extracellular microenvironment. See, Schlessinger and Ullrich, 1992, Neuron, 9:1-20.

The close homology of the intracellular regions of KDR/FLK-1 with that of the PDGF-β receptor (50.3% homology) and/or the related flt-I receptor indicates the induction of overlapping signal transduction pathways. For example, for the PDGF-β receptor, members of the src family (Twamley et al., 1993, Proc. Natl. Acad. Sci. USA, 90:7696-7700), phosphatidylinositol-3′-kinase (Hu et al., 1992, Mol. Cell. Biol., 12:981-990), phospholipase cy (Kashishian & Cooper, 1993, Mol. Cell. Biol., 4:49-51), ras-GTPase-activating protein, (Kashishian et al., 1992, EMBO J., 11:1373-1382), PTP-ID/syp (Kazlauskas et al., 1993, Proc. Natl. Acad. Sci. USA, 10 90:6939-6943), Grb2 (Arvidsson et al., 1994, Mol. Cell. Biol., 14:6715-6726), and the adapter molecules Shc and Nck (Nishimura et al., 1993, Mol. Cell. Biol., 13:6889-6896), have been shown to bind to regions involving different autophosphorylation sites. See generally, Claesson-Welsh, 1994, Prog. Growth Factor Res., 5:37-54. Thus, it is likely that signal transduction pathways activated by KDR/FLK-1 include the ras pathway (Rozakis et al., 1992, Nature, 360:689-692), the PI-3′-kinase, the src-mediated and the plcy-mediated pathways. Each of these pathways may play a critical role in the angiogenic and/or vasculogenic effect of KDR/FLK-1 in endothelial cells. Consequently, a still further aspect of this invention relates to the use of the organic compounds described herein to modulate angiogenesis and vasculogenesis as such processes are controlled by these pathways.

Conversely, disorders related to the shrinkage, contraction or closing of blood vessels, such as restenosis, are also implicated and may be treated or prevented by the methods of this invention.

Fibrotic disorders refer to the abnormal formation of extracellular matrices. Examples of fibrotic disorders include hepatic cirrhosis and mesangial cell proliferative disorders. Hepatic cirrhosis is characterized by the increase in extracellular matrix constituents resulting in the formation of a hepatic scar. An increased extracellular matrix resulting in a hepatic scar can also be caused by a viral infection such as hepatitis. Lipocytes appear to play a major role in hepatic cirrhosis. Other fibrotic disorders implicated include atherosclerosis.

Mesangial cell proliferative disorders refer to disorders brought about by abnormal proliferation of mesangial cells. Mesangial proliferative disorders include various human renal diseases such as glomerulonephritis, diabetic nephropathy and malignant nephrosclerosis as well as such disorders as thrombotic microangiopathy syndromes, transplant rejection, and glomerulopathies. The RTK PDGFR has been implicated in the maintenance of mesangial cell proliferation. Floege et al., 1993, Kidney International 43:47 S-54S.

Many cancers are cell proliferative disorders and, as noted previously, PKs have been associated with cell proliferative disorders. Thus, it is not surprising that PKs such as, for example, members of the RTK family have been associated with the development of cancer. Some of these receptors, like EGFR (Tuzi et al., 1991, Br. J. Cancer 63:227-233, Torp et al., 1992, APMIS 100:713-719) HER2/neu (Slamon et al., 1989, Science 244:707-712) and PDGF-R (Kumabe et al., 1992, Oncoqene, 7:627-633) are over-expressed in many tumors and/or persistently activated by autocrine loops. In fact, in the most common and severe cancers these receptor over-expressions (Akbasak and Suner-Akbasak et al., 1992, J. Neurol. Sci., 111:119-133, Dickson et al., 1992, Cancer Treatment Res. 61:249-273, Korc et al., 1992, J. Clin. Invest. 90:1352-1360) and autocrine loops (Lee and Donoghue, 1992, J. Cell. Biol., 118:1057-1070, Korc et al., supra, Akbasak and Suner-Akbasak et al., sura) have been demonstrated. For example, EGFR has been associated with squamous cell carcinoma, astrocytoma, glioblastoma, head and neck cancer, lung cancer and bladder cancer. HER2 has been associated with breast, ovarian, gastric, lung, pancreas and bladder cancer. PDGFR has been associated with glioblastoma and melanoma as well as lung, ovarian and prostate cancer. The RTK c-met has also been associated with malignant tumor formation. For example, c-met has been associated with, among other cancers, colorectal, thyroid, pancreatic, gastric and hepatocellular carcinomas and lymphomas. Additionally c-met has been linked to leukemia. Over-expression of the c-met gene has also been detected in patients with Hodgkins disease and Burkitts disease.

The association between abnormal PK activity and disease is not restricted to cancer. For example, RTKs have been associated with diseases such as psoriasis, diabetes mellitus, endometriosis, angiogenesis, atheromatous plaque development, Alzheimer's disease, restenosis, von Hippel-Lindau disease, epidermal hyperproliferation, neurodegenerative diseases, age-related macular degeneration and hemangiomas. For example, EGFR has been indicated in corneal and dermal wound healing. Defects in Insulin-R and IGF-1R are indicated in type-II diabetes mellitus. A more complete correlation between specific RTKs and their therapeutic indications is set forth in Plowman et al., 1994, DN&P 7:334-339.

Pharmaceutical Compositions and Use

A compound of the present invention or a physiologically acceptable salt thereof, can be administered as such to a human patient or can be administered in pharmaceutical compositions in which the foregoing materials are mixed with suitable carriers or excipient(s). Techniques for formulation and administration of drugs may be found in “Remington's Pharmacological Sciences,” Mack Publishing Co., Easton, Pa., latest edition.

Routes of Administration

Suitable routes of administration may include, without limitation, oral, intraoral, rectal, transmucosal or intestinal administration or intramuscular, epicutaneous, parenteral, subcutaneous, transdermal, intramedullary, intrathecaly, direct intraventricular, intravenous, intravitreal, intraperitoneal, intranasal, intramuscular, intradural, intrarespiratory, nasal inhalation or intraocular injections. The preferred routes of administration are oral and parenteral. Alternatively, one may administer the compound in a local rather than systemic manner, for example, via injection of the compound directly into a solid tumor, often in a depot or sustained release formulation. Furthermore, one may administer the drug in a targeted drug delivery system, for example, in a liposome coated with tumor-specific antibody. The liposomes will be targeted to and taken up selectively by the tumor.

Composition/Formulation

Pharmaceutical compositions of the present invention may be manufactured by processes well known in the art, e.g., by means of conventional mixing, dissolving, granulating, dragee-making, levigating, emulsifying, encapsulating, entrapping, lyophilizing processes or spray drying. Pharmaceutical compositions for use in the methods of the present invention may be prepared by any methods of pharmacy, but all methods include the step of bringing in association the active ingredient with the carrier which constitutes one or more necessary ingredients. In particular, pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

Dosage forms include tablets, troches, dispersions, suspensions, solutions, capsules, patches, syrups, elixirs, gels, powders, magmas, lozenges, ointments, creams, pastes, plasters, lotions, discs, suppositories, nasal or oral sprays, aerosols and the like.

For injection, the compounds of the invention may be formulated in aqueous solutions, preferably in physiologically compatible buffers such buffers with or without a low concentration of surfactant or cosolvent, or physiological saline buffer. For transmucosal administration, penetrants appropriate to the barrier to be permeated are used in the formulation. Such penetrants are generally known in the art.

For oral administration, the compounds can be formulated by combining the active compounds with pharmaceutically acceptable carriers well known in the art. Such carriers enable the compounds of the invention to be formulated as tablets, pills, lozenges, dragees, capsules, liquids, gels, syrups, slurries, suspensions and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars, including lactose, sucrose, mannitol, or sorbitol, cellulose preparations such as, for example, maize starch, wheat starch, rice starch and potato starch and other materials such as gelatin, gum tragacanth, methyl cellulose, hydroxypropylmethyl-cellulose, sodium carboxymethylcellulose, and/or polyvinyl-pyrrolidone (PVP). If desired, disintegrating agents may be added, such as cross-linked polyvinyl pyrrolidone, agar, or alginic acid. A salt such as sodium alginate may also be used.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with a filler such as lactose, a binder such as starch, and/or a lubricant such as talc or magnesium stearate and, optionally, stabilizers. In soft capsules, the active compounds may be dissolved or suspended in suitable liquids, such as fatty oils, liquid paraffin, liquid polyethylene glycols, cremophor, capmul, medium or long chain mono- di- or triglycerides. Stabilizers may be added in these formulations, also.

For administration by inhalation, the compounds for use according to the present invention are conveniently delivered in the form of an aerosol spray using a pressurized pack or a nebulizer and a suitable propellant, e.g., without limitation, dichlorodifluoromethane, trichlorofluoromethane, dichlorotetrafluoroethane or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of the compound and a suitable powder base such as lactose or starch.

The compounds may also be formulated for parenteral administration, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulating materials such as suspending, stabilizing and/or dispersing agents.

Pharmaceutical compositions for parenteral administration include aqueous solutions of a water soluble form, such as, without limitation, a salt, of the active compound. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances which increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions.

Alternatively, the active ingredient may be in powder form for constitution with a suitable vehicle, e.g., sterile, pyrogen-free water, before use.

The compounds may also be formulated in rectal compositions such as suppositories or retention enemas, using, e.g., conventional suppository bases such as cocoa butter or other glycerides.

In addition to the formulations described previously, the compounds may also be formulated as depot preparations. Such long acting formulations may be administered by implantation (for example, subcutaneously or intramuscularly) or by intramuscular injection. A compound of this invention may be formulated for this route of administration with suitable polymeric or hydrophobic materials (for instance, in an emulsion with a pharmacologically acceptable oil), with ion exchange resins, or as a sparingly soluble derivative such as, without limitation, a sparingly soluble salt.

Alternatively, other delivery systems for hydrophobic pharmaceutical compounds may be employed. Liposomes and emulsions are well known examples of delivery vehicles or carriers for hydrophobic drugs. In addition, certain organic solvents such as dimethylsulfoxide also may be employed, although often at the cost of greater toxicity.

Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions herein also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Many of the PK modulating compounds of the invention may be provided as physiologically acceptable salts wherein the claimed compound may form the negatively or the positively charged species. Examples of salts in which the compound forms the positively charged moiety include, without limitation, quaternary ammonium (defined elsewhere herein), salts such as the hydrochloride, sulfate, carbonate, lactate, tartrate, maleate, succinate, malate, acetate and methylsulfonate (CH₃SO₃), wherein the nitrogen atom of the quaternary ammonium group is a nitrogen of the selected compound of this invention which has reacted with the appropriate acid. Salts in which a compound of this invention forms the negatively charged species include, without limitation, the sodium, potassium, calcium and magnesium salts formed by the reaction of a carboxylic acid group in the compound with an appropriate base (e.g. sodium hydroxide (NaOH), potassium hydroxide (KOH), Calcium hydroxide (Ca(OH)₂), etc.).

Dosage

Pharmaceutical compositions suitable for use in the present invention include compositions wherein the active ingredients are contained in an amount sufficient to achieve the intended purpose, i.e., the modulation of PK activity or the treatment or prevention of a PK-related disorder.

More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

For any compound used in the methods of the invention, the therapeutically effective amount or dose can be estimated initially from cell culture assays. Then, the dosage can be formulated for use in animal models so as to achieve a circulating concentration range that includes the IC₅₀ as determined in cell culture (i.e., the concentration of the test compound which achieves a half-maximal inhibition of PDGFR activity). Such information can then be used to more accurately determine useful doses in humans.

Toxicity and therapeutic efficacy of the compounds described herein can be determined by standard pharmaceutical procedures in cell cultures or experimental animals, e.g., by determining the IC₅₀ and the LD₅₀ (both of which are discussed elsewhere herein) for a subject compound. The data obtained from these cell culture assays and animal studies can be used in formulating a range of dosage for use in humans. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide plasma levels of the active species which are sufficient to maintain the kinase modulating effects. These plasma levels are referred to as minimal effective concentrations (MECs). The MEC will vary for each compound but can be estimated from in vitro data, e.g., the concentration necessary to achieve 50-90% inhibition of a kinase may be ascertained using the assays described herein. Dosages necessary to achieve the MEC will depend on individual characteristics and route of administration. HPLC assays or bioassays can be used to determine plasma concentrations.

Dosage intervals can also be determined using MEC value. Compounds should be administered using a regimen that maintains plasma levels above the MEC for 10-90% of the time, preferably between 30-90% and most preferably between 50-90%. Typically, therapeutically effective amounts of compounds of the invention may range from approximately 10 mg/m² to 1000 mg/m² per day, preferably 25 mg/m² to 500 mg/m² per day.

In cases of local administration or selective uptake, the effective local concentration of the drug may not be related to plasma concentration and other procedures known in the art may be employed to determine the correct dosage amount and interval.

The amount of a composition administered will, of course, be dependent on the subject being treated, the severity of the affliction, the manner of administration, the judgment of the prescribing physician, etc.

### EXAMPLES

The following preparations and examples are given to enable those skilled in the art to more clearly understand and to practice the present invention. They should not be considered as limiting the scope of the invention, but merely as being illustrative and representative thereof.

General Synthetic Procedure

The following general methodology may be employed to prepare the compounds of this invention:

It will be appreciated by those skilled in the art that other synthetic pathways for forming the compounds of the invention are available and that the following is offered by way of example and not limitation. 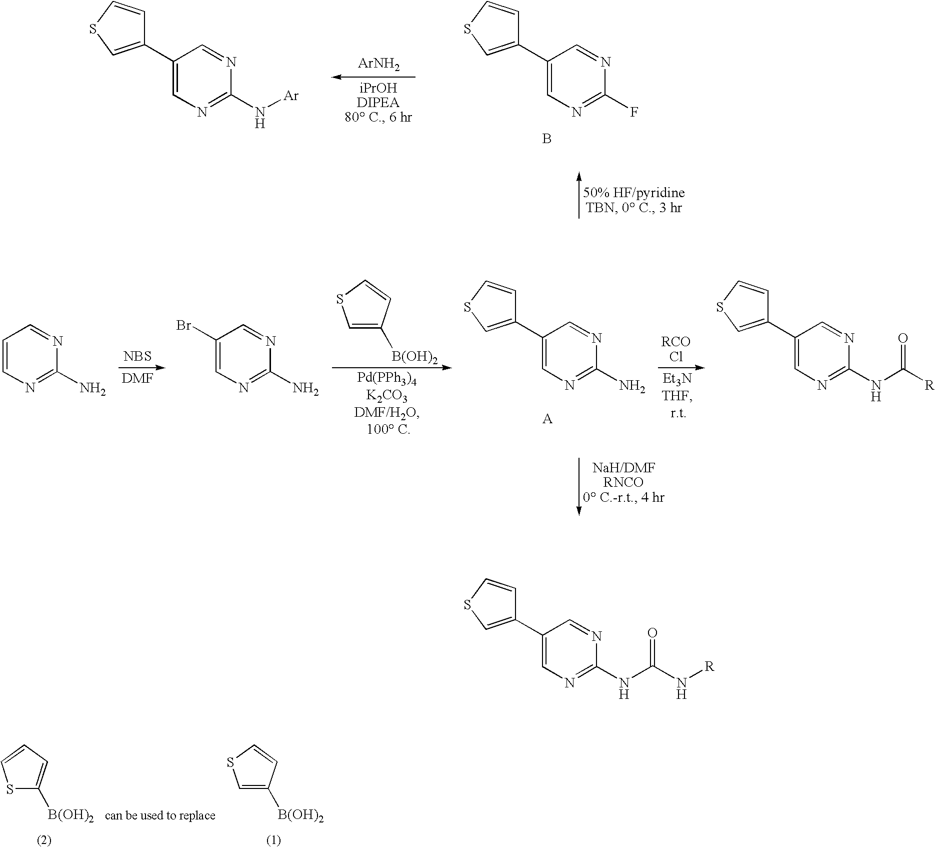
Compounds I-18 were made by the methods in this scheme.

### 5-Thiophen-3-yl-pyrimidin-2-ylamine (A1)

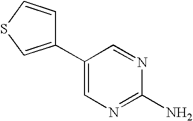

2-amino-pyrimidine (1.9 g, 20 mmol) was suspended in 40 mL of dichloromethane and 40 mL of acetonitrile. N-Bromosuccinimide (5.34 g, 30 mmol) was added with stirring. The mixture was stirred for 72 hours at room temperature. The mixture was washed with sodium bisulfite solution, water and chloroform. The precipitate was collected by vacuum filtration and washed with acetone. The solids were dried under vacuum to give 3.2 g (91% yield) of 2-amino-5-bromo-pyrimidine as a white solid. ¹H-NMR (dimethylsulfoxide-d₆) δ 8.28 (s, 2H, aromatic), 6.87 (s, 2H, NH₂). MS (m/z) 174 [M+1].

2-Amino-5-bromo-pyrimidine (710 mg, 4.1 mmol), 780 mg (6.1 mmol) of thiophene-3-boronic acid, 375 mg (0.33 mmol) of tetrakis(triphenylphosphine)palladium(0), 1700 mg (12.3 mmol) of potassium carbonate, 12 mL of dimethylformamide and 1 mL of water were heated overnight at 100° C. The mixture was cooled and vacuum filtered to remove the inorganic precipitate. The solvent was removed by rotary evaporation and the residue purified by flash chromatography on silica gel eluting with dichlomethane:methanol 30:1 to give 5-thiophen-3-yl-pyrimidin-2-ylamine. ¹H-NMR (dimethylsulfoxide-d₆) δ 8.60 (s, 2H, aromatic), 7.75 (m, 1H, aromatic), 7.61 (m, 1H, aromatic), 7.50 (dd, 1H, aromatic), 6.70 (br s, 2H, NH₂). MS (m/z) 178 [M+1].

### 5-Thiophen-2-yl-pyrimidin-2-ylamine (A2)

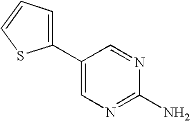

Using thiophene-2-boronic acid to replace thiophene-3-boronic acid, the title compound was made following a similar method as described for the synthesis of Al. ¹H-NMR (dimethylsulfoxide-d₆) δ 8.51 (s, 2H), 7.44 (dd, 1H), 7.34 (dd, 1H), 7.08 (dd, 1H), 6.87 (brs, 2H). MS (m/z) 178 [M+1].

### 2-Fluoro-5-thiorhen-3-yl-pyrimidine (B1)

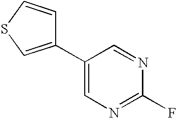

5-Thiophen-3-yl-pyrimidin-2-ylamine (400 mg, 2.3 mmol) was dissolved in 1.2 mL of dry pyridine and cooled to −70° C. Hydrogen fluoride (70% in pyridine, 3.1 mL) was slowly added keeping the temperature below −30° C. tert-Butyl nitrite (0.2 mL, 3.2 mmol) was slowly added at −40° C. and the mixture stirred for 5 minutes. The mixture was allowed to warm to 0° C. and stirred for 1 hour. The mixture was dropped carefully into 40 mL mixture of saturated sodium bicarbonate solution and ice. The pH was adjusted to 9 with saturated sodium bicarbonate solution and the mixture extracted with 50 mL of chloroform. The chloroform extract was dried over anhydrous sodium sulfate and rotary evaporated to dryness to give 2-fluoro-5-thiophen-3-yl-pyrimidine. ¹H-NMR (CDCl₃) δ 8.78 (s, 2H, aromatic), 7.56 (m, 1H, aromatic), 7.45 (m, 1H, aromatic), 7.28 (m, 1H, aromatic).

### 2-Fluoro-5-thiophen-2-yl-pyrimidine (B2)

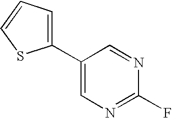

The title compound is made from 5-thiophen-2-yl-pyrimidin-2-ylamine following the similar method for making 2-fluoro-5-thiophen-3-yl-pyrimidine. ¹H-NMR (CDCl₃) δ 8.71 (s, 2H), 7.34 (m, 1H), 7.26 (m, 1H), 7.06 (dd, 1H).

### Example 1

### 4-(5-Thiophen-3-yl-pyrimidin-2-ylamino)-phenol

2-Fluoro-5-thiophen-3-yl-pyrimidine (75 mg, 0.41 mmol), 112 mg (1.03 mmol) of 4-aminophenol and 0.19 mL (1.10 mmol) of diisopropylethylamine in 3 mL of isopropanol were heated at 70° C. overnight. The solvent was removed by rotary evaporation and the residue purified by flash chromatography on silica gel eluting with dichloromethane:methanol 80:1 and then dichloromethane:methanol 60:1 to give 4-(5-thiophen-3-yl-pyrimidin-2-ylamino)-phenol. ¹H-NMR (dimethylsulfoxide-d₆) δ 9.38 (s, 1H), 9.02 (s, 1H), 8.77 (s, 2H), 7.84 (m, 1H), 7.66 (m, 1H), 7.56 (dd, 1H), 7.48 (m, 2H), 6.68 (m, 2H).

### Example 2

### N-[4-(5-Thiophen-3-yl-pyrimidin-2-ylamino)-phenyl]-acetamide

2-Fluoro-5-thiophen-3-yl-pyrimidine (75 mg, 0.41 mmol), 155 mg (1.03 mmol) of 4-N-acetyl-1,4-diaminobenzene and 0.19 mL (1.10 mmol) of diisopropylethylamine in 3 mL of isopropanol were heated at 70° C. overnight. The solvent was removed by rotary evaporation and the residue purified by flash chromatography on silica gel eluting with dichloromethane:methanol 50:1 and then dichloromethane:methanol 30:1 to give N-[4-(5-thiophen-3-yl-pyrimidin-2-ylamino)-phenyl]-acetamide. H-NMR (dimethylsulfoxide-d₆) δ 9.78 (s, 1H), 9.63 (s, 1H), 8.83 (s, 2H), 7.88 (m, 1H), 7.65 (multiplets, 2H), 7.58 (dd, 1H), 7.45 (m, 1H), 6.68 (m, 2H), 2.00 (s, 3H). MS (m/z) 310 [M+].

### Example 3

### (4-Morpholin-4-yl-phenyl)-(5-thiophen-3-yl-pyrimidin-2-yl)-amine

2-Fluoro-5-thiophen-3-yl-pyrimidine (120 mg, 0.66 mmol), 352 mg (2.0 mmol) of 4-(morpholin-4-yl)-aniline and 0.23 mL (1.4 mmol) of diisopropylethylamine in 4 mL of isopropanol were heated at 90° C. overnight. The solvent was removed by rotary evaporation and the residue purified by flash chromatography on silica gel eluting with dichloromethane:methanol 80:1, 70:1 and then 50:1 to give (4-morpholin-4-yl-phenyl)-(5-thiophen-3-yl-pyrimidin-2-yl)-amine. ¹H-NMR (dimethylsulfoxide-d₆) δ 9.42 (s, 1H), 8.74 (s, 2H), 7.80 (m, 1H), 7.60 (m, 1H), 7.54 (multiplets, 3H), 6.84 (m, 2H), 3.68 (m, 4H), 2.95 (m, 4H). MS (m/z) 338 [M+].

### Example 4

### 4-Amino-N-[4-(5-thiophen-3-yl-pyrimidin-2-ylamino)-phenyl]-benzamide

2-Fluoro-5-thiophen-3-yl-pyrimidine (120 mg, 0.66 mmol), 449 mg (2.0 mmol) of 4,4′-diaminobenzanilide and 0.23 mL (1.4 mmol) of diisopropylethylamine in 4 mL of isopropanol were heated at 90° C. overnight. The solvent was removed by rotary evaporation and the residue purified by flash chromatography eluting with dichloromethane:methanol 40:1 then 30:1 and then 20:1 to give 4-amino-N-[4-(5-thiophen-3-yl-pyrimidin-2-ylamino)-phenyl]-benzamide. ¹H-NMR (dimethylsulfoxide-d₆) δ 9.65 (s, 2H), 8.83 (s, 2H), 7.89 (m, 1H), 7.65 (multiplets, 8H), 6.58 (m, 2H), 5.70 (s, 2H). MS (m/z) 387 [M+].

### Example 5

### (4-Methoxy-phenyl)-(5-thiophen-3-yl-pyrimidin-2-yl)-amine

2-Fluoro-5-thiophen-3-yl-pyrimidine (100 mg, 0.55 mmol), 203 mg (1.65 mmol) of 4-methoxyaniline and 0.19 mL (1.10 mmol) of diisopropylethylamine in 2 mL of isopropanol were heated at 80° C. for 6 hours. The solvent was removed by rotary evaporation and the residue purified by flash chromatography on silica gel eluting with dichloromethane:methanol 80:1 and then dichloromethane:methanol 60:1 to give (4-methoxy-phenyl)-(5-thiophen-3-yl-pyrimidin-2-yl)-amine. ¹H-NMR (dimethylsulfoxide-d₆) δ 9.52 (s, 1H), 8.80 (s, 2H), 7.86 (m, 1H, aromatic), 7.60 (multiplets, 4H), 6.86 (d, 2H), 3.70 (s, 3H). MS (m/z) 284 [M+1].

### Example 6

### N-(5-Thiophen-3-yl-pyrimidin-2-yl)-benzene-1,3-diamine

2-Fluoro-5-thiophen-3-yl-pyrimidine (100 mg, 0.55 mmol)), 178 mg of benzene-1,3-diamine (1.65 mmol) and 0.19 mL of diisopropylethylamine (1.10 mmol) in 2 mL of isopropanol were heated at 80° C. for 6 hours. The solvent was removed by rotary evaporation and the residue purified by flash chromatography on silica gel eluting with dichloromethane:methanol 80:1 and then dichloromethane:methanol 60:1 to give N-(5-thiophen-3-yl-pyrimidin-2-yl)-benzene-1,3-diamine. ¹H-NMR (dimethylsulfoxide-d₆) δ 9.40 (s, 1H), 8.80 (s, 2H), 7.88 (m, 1H), 7.65 (m, 1H), 7.57 (m, 1H), 7.04 (m, 1H), 6.87 (m, 2H), 6.18 (m, 1H, NH), 4.93 (br s, 2H, NH₂). MS (m/z) 269 [M+1].

### Example 7

### 3-(5-Thiophen-3-yl-pyrimidin-2-ylamino)-benzoic acid

2-Fluoro-5-thiophen-3-yl-pyrimidine (100 mg, 0.55 mmol)), 175 mg of 3-amino-benzoic acid (1.65 mmol) and 0.19 mL of diisopropylethylamine (1.10 mmol) in 2 mL of isopropanol were heated at 80° C. for 6 hours. The solvent was removed by rotary evaporation and the residue purified by flash chromatography on silica gel eluting with dichloromethane:methanol:water 40:1:0.1 to give 3-(5-thiophen-3-yl-pyrimidin-2-ylamino)-benzoic acid. ¹H-NMR (dimethylsulfoxide-d₆) δ 12.80 (br s, 1H), 9.93 (s, 1H,), 8.91 (s, 2H), 8.46 (m, 1H), 8.46 (m, 1H), 7.95 (multiplets, 2H), 7.68 (m, 1H), 7.52 (m, 1H), 7.49 (m, 1H). MS (m/z) 296 [M−1].

### Example 8

### N-(5-Thiophen-3-yl-pyrimidin-2-yl)-benzene-1,4-diamine

2-Fluoro-5-thiophen-3-yl-pyrimidine (100 mg, 0.55 mmol)), 175 mg of 1,4-phenylene-diamine

(1.65 mmol) and 0.19 mL of diisopropylethylamine (1.10 mmol) in 2 mL of isopropanol were heated at 80° C. for 6 hours. The solvent was removed by rotary evaporation and the residue purified by flash chromatography on silica gel eluting with dichloromethane:methanol 70:1 and then dichloromethane:methanol 60:1 to give N-(5-thiophen-3-yl-pyrimidin-2-yl)-benzene-1,4-diamine. ¹H-NMR (dimethylsulfoxide-d₆) δ 9.20 (s, 1H), 8.72 (s, 2H), 7.82 (m, 1H), 7.63 (m, 1H), 7.53 (dd, 1H), 7.32 (d, 2H), 6.50 (d, 2H), 4.74 (s, 2H). MS (m/z) 269 [M+1].

### Example 9

### 1-(4-Methoxy-phenyl)-3-(5-thiophen-3-yl-pyrimidin-2-yl)-urea

2-Amino-5-thiophen-3-yl-pyrimidine (A1) (88 mg, 0.50 mmol) in 1 mL of dimethylformamide was treated with 20 mg (0.50 mmol) of sodium hydride in 0.5 mL of dimethylformamide at 0° C. 4-Methoxyphenylisocyanate (62 mg, 0.55 mmol) was added at 0° C. and the mixture allowed to warm to room temperature and stirred for 3 hours. The white solid was collected by vacuum filtration and washed with 1 mL of methanol to give 1-(4-methoxy-phenyl)-3-(5-thiophen-3-yl-pyrimidin-2-yl)-urea. ¹H-NMR (dimethylsulfoxide-d₆) δ 11.18 (br s, 1H), 10.15 (br s, 1H), 9.03 (s, 2H), 8.03 (s, 1H), 7.72 (m, 1H), 7.63 (m, 1H), 7.46 (d, 2H), 6.95 (d, 2H), 3.73 (s, 3H). MS (m/z) 327 [M+1].

### Example 10

### 1-(4-Fluoro-phenyl)-3-(5-thiophen-3-yl-pyrimidin-2-yl)-urea

2-Amino-5-thiophen-3-yl-pyrimidine (A1) (177 mg, 1.0 mmol) in 2 mL of dimethylformamide was treated with 40 mg (1.0 mmol) of sodium hydride in 1 mL of dimethylformamide at 0° C. 4-Fluorophenylisocyanate (103 mg, 1.1 mmol) in 0.2 mL of dimethylformamide was added at 0° C. and the mixture allowed to warm to room temperature and stirred for 3 hours. The white solid was collected by vacuum filtration and washed with 1 mL of methanol to give 1-(4-fluoro-phenyl)-3-(5-thiophen-3-yl-pyrimidin-2-yl)-urea. ¹H-NMR (dimethylsulfoxide-d₆) δ 9.06 (s, 1H), 9.04 (s, 2H), 8.07 (m, 0.5H), 8.02 (m, 1H), 7.72 (m, 1H), 7.68 (m, 0.5H), 7.64 (m, 1H), 7.60 (m, 2H), 7.17 (m, 2H).

### Example 11

### 4-(4-Methyl-piperazin-1-ylmethyl)-N-[3-(5-thiophen-3-yl-pyrimidin-2-ylamino)-phenyl]-benzamide

4-Chloromethylbenzoic acid (1.7 g, 10 mmol) and 2.78 mL (20 mmol) of triethylamine in 50 mL of dimethylformamide were stirred with 1.22 mL (11 mmol) of N-methylpiperazine overnight at room temperature. The white solid was collected by vacuum filtration and washed with dichloromethane. The combined filtrates were rotary evaporated to dryness to give 4-(4-methyl-piperazin-1-ylmethyl)-benzoic acid. N-(5-Thiophen-3-yl-pyrimidin-2-yl)-benzene-1,3-diamine (40 mg, 0.15 mmol), triethylamine (0.112 mL, 0.80 mmol), BOP reagent (265 mg, 0.6 mmol) and 93 mg (0.4 mmol) of 4-(4-methyl-piperazin-1-ylmethyl)-benzoic acid in 1 mL of dimethylformamide were stirred overnight at room temperature. The solvent was evaporated and the residue purified by flash chromatography eluting with dichloromethane:methanol 20:1 and then dichloromethane:methanol 15:1 to give of 4-(4-methyl-piperazin-1-ylmethyl)-N-[3-(5-thiophen-3-yl-pyrimidin-2-ylamino)-phenyl]-benzamide. ¹H-NMR (dimethylsulfoxide-d₆) δ 10.11 (s, 1H), 9.68 (s, 1H), 8.80 (s, 2H), 7.85 (m, 3H), 7.62 (m, 1H), 7.57 (m, 1H), 7.42 (d, 1H), 7.38 (d, 2H), 7.26 (d, 1H), 7.18 (m, 1H), 3.45 (s, 2H), 2.50 (m, 8H), 2.30 (s, 3H). MS (m/z)-485 [M+1].

### Example 12

### 4-(4-Methyl-piperazin-1-ylmethyl)-N-[4-(5-thiophen-3-yl-pyrimidin-2-ylamino)-phenyl]-benzamide

4-Chloromethylbenzoic acid (1.7 g, 10 mmol) and 2.78 mL (20 mmol) of triethylamine in 50 mL of dimethylformamide was stirred with 1.22 mL (11 mmol) of N-methylpiperazine overnight at room temperature. The white solid was collected by vacuum filtration and washed with dichloromethane. The combined filtrates were rotary evaporated to dryness to give 4-(4-methyl-piperazin-1-ylmethyl)-benzoic acid. N-(5-Thiophen-3-yl-pyrimidin-2-yl)-benzene-1,4-diamine (55 mg, 0.20 mmol), triethylamine (0.112 mL, 0.80 mmol), BOP reagent (265 mg, 0.6 mmol) and 93 mg (0.4 mmol) of 4-(4-methyl-piperazin-1-ylmethyl)-benzoic acid in 1 mL of dimethylformamide were stirred overnight at room temperature. The solvent was evaporated and the residue purified by flash chromatography eluting with dichloromethane:methanol 20:1 and then dichlormehane:methanol 15:1 to give 4-(4-methyl-piperazin-1-ylmethyl)-N-[4-(5-thiophen-3-yl-pyrimidin-2-ylamino)-phenyl]-benzamide. ¹H-NMR (dimethylsulfoxide-d₆) δ 10.03 (s, 1H), 9.65 (s, 1H), 8.80 (s, 2H), 7.85 (m, 3H), 7.67 (m, 2H), 7.63 (m, 2H), 7.57 (d, 1H), 7.38 (d, 2H), 3.45 (s, 2H), 2.50 (m, 8H), 2.30 (s, 3H). MS (m/z) 485 [M+1].

### Example 13

### N-[4-(5-Thiophen-3-yl-pyrimidin-2-ylamino)-phenyl]-acetamide

2-Fluoro-5-thiophen-2-yl-pyrimidine (110 mg, 0.55 mmol), 247 mg of N-(4-aminophenyl)-acetamide (1.65 mmol) and 0.19 mL of diisopropylethylamine (1.10 mmol) in 3 mL of isopropanol were heated at 90° C. for 6 hours. The solvent was removed by rotary evaporation and the residue purified by flash chromatography on silica gel eluting with dichloromethane:methanol 60:1 to give N-[4-(5-thiophen-3-yl-pyrimidin-2-ylamino)-phenyl]-acetamide. ¹H-NMR (dimethylsulfoxide-d₆) δ 9.80 (s, 1H), 9.73 (s, 1H), 8.73 (s, 2H), 7.63 (m, 2H), 7.53 (m, 1H), 7.48 (m, 3H), 7.14 (br s, 1H), 2.00 (s, 3H). MS (m/z) 311 [M+1].

### Example 14

### N-[3-(5-Thiophen-3-yl-pyrimidin-2-ylamino)-phenyl]-acetamide

2-Fluoro-5-thiophen-2-yl-pyrimidine (110 mg, 0.55 mmol), 247 mg of N-(3-aminophenyl)-acetamide (1.65 mmol) and 0.19 mL of diisopropylethylamine (1.10 mmol) in 3 mL of isopropanol were heated at 90° C. for 6 hours. The solvent was removed by rotary evaporation and the residue purified by flash chromatography on silica gel eluting with dichloromethane:methanol 60:1 to give N-[3-(5-thiophen-3-yl-pyrimidin-2-ylamino)-phenyl]-acetamide. ¹H-NMR (dimethylsulfoxide-d₆) δ 9.85 (s, 1H), 9.81 (s, 1H), 8.73 (s, 2H), 7.93 (m, 1H), 7.55 (d, 1H), 7.50 (d, 1H), 7.39 (d, 1H), 7.23 (d, 1H), 7.15 (m, 2H), 2.05 (s, 3H). MS (m/z) 311 [M+1].

### Example 15

### 4-(5-Thiophen-3-yl-pyrimidin-2-ylamino)-phenol

2-Fluoro-5-thiophen-2-yl-pyrimidine (110 mg, 0.55 mmol), 180 mg of 4-aminophenol (1.65 mmol) and 0.19 mL of diisopropylethylamine (1.10 mmol) in 3 mL of isopropanol were heated at 90° C. for 6 hours. The solvent was removed by rotary evaporation and the residue purified by flash chromatography on silica gel eluting with dichloromethane:methanol 60:1 to give 4-(5-thiophen-3-yl-pyrimidin-2-ylamino)-phenol. ¹H-NMR (dimethylsulfoxide-d₆) δ 8.58 (br s, 1H), 8.47 (s, 2H), 8.20 (br s, 1H), 7.35 (m, 2H), 7.19 (dd, 1H), 7.11 (dd, 1H), 6.99 (m, 1H), 6.71 (m. 1H), 6.69 (m, 1H). MS (m/z)268 [M−1].

### Example 16

### 4-Amino-N[4-(5-thiophen-3-yl-pyrimidin-2-ylamino)-phenyl]-benzamide

2-Fluoro-5-thiophen-2-yl-pyrimidine (110 mg, 0.55 mmol), 374 mg of 4-amino-N-(4-amino-phenyl)-benzamide (1.65 mmol) and 0.19 mL of diisopropylethylamine (1.10 mmol) in 3 mL of isopropanol were heated at 90° C. for 6 hours. The solvent was removed by rotary evaporation and the residue purified by flash chromatography on silica gel eluting with dichloromethane:methanol 60:1 to give 4-amino-N-[4-(5-thiophen-3-yl-pyrimidin-2-ylamino)-phenyl]-benzamide. ¹H-NMR (dimethylsulfoxide-d₆) δ 9.85 (s, 1H), 9.76 (s, 1H), 8.85 (s, 2H), 7.77 (m, 6H), 7.59 (dd, 2H), 7.25 (dd, 1H), 6.68 (dd, 2H), 5.80 (s, 2H). MS (m/z) 388 [M+1].

### Example 17

### 3-(5-Thiophen-3-yl-pyrimidin-2-ylamino)-benzoic acid

2-Fluoro-5-thiophen-2-yl-pyrimidine (110 mg, 0.55 mmol), 226 mg of 3-aminobenzoic acid (1.65 mmol) and 0.19 mL of diisopropylethylamine (1.10 mmol) in 3 mL of isopropanol were heated at 90° C. for 6 hours. The solvent was removed by rotary evaporation and the residue purified by flash chromatography on silica gel eluting with dichloromethane:methanol:water 30:1:0.1 to give 3-(5-thiophen-3-yl-pyrimidin-2-ylamino)-benzoic acid. ¹H-NMR (dimethylsulfoxide-d₆) δ12.82 (br s, 1H), 10.00 (s, 1H), 8.80 (s, 2H), 8.40 (m, 1H), 7.95 (m, 1H), 7.53 (m, 3H), 7.40 (m, 1H), 7.15 (m, 1H). MS (m/z) 298 [M+1].

### Example 18

### N-Phenyl-3-(5-thiophen-3-yl-pyrimidin-2-ylamino)-benzamide

3-(5-Thiophen-2-yl-pyrimidin-2-ylamino)-benzoic acid, 0.027 mL (0.30 mmol) of aniline, triethylamine (0.042 mL, 0.30 mmol), BOP reagent (88 mg, 0.2 mmol) in 1 mL of dimethylformamide were stirred overnight at room temperature. The solvent was evaporated and the residue triturated in refluxing methanol, collected by vacuum filtration and washed with methanol to give N-phenyl-3-(5-thiophen-3-yl-pyrimidin-2-ylamino)-benzamide. ¹H-NMR (dimethylsulfoxide-d₆) δ 10.20 (s, 1H), 10.02 (s, 1H), 8.81 (s, 2H), 8.29 (s, 1H), 7.95 (d, 1H), 7.76 (d, 2H), 7.55 (d, 1H), 7.52 (m, 2H), 7.43 (t, 1H), 7.32 (t, 2H), 7.25 (dd, 1H), 7.08 (t, 1H). MS (m/z) 373 [M+1].

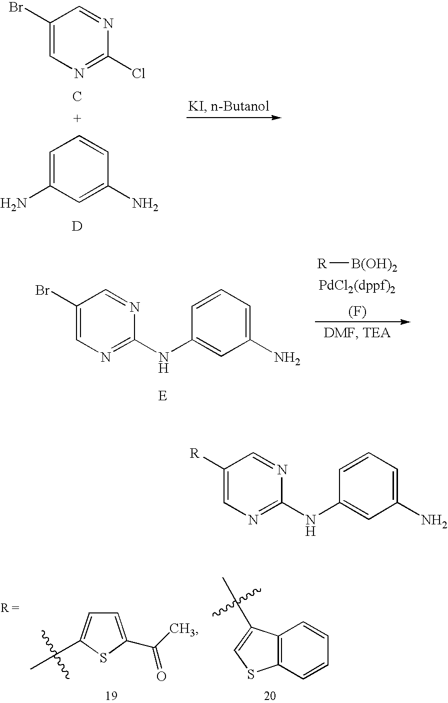

### N-(5-Bromo-pyrimidin-2-yl)-benzene-1,3-diamine (E)

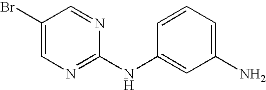

A stirring reaction mixture of C (4.57 g, 23.6 mmol), D (15.30 g, 140.0 mmol) and KI (3.92 g, 23.6 mmol) in n-butanol (40 mL) was heated to 80° C. for 4 h. The mixture was cool down to ambient temperature then was diluted with MeOH (10 mL) and filtered. The filtrate was evaporated to dryness under vacuum. The residue was purified using column chromatography (silica gel, 1:1:1 hexane/CH₂Cl₂/EtOAc). The fractions containing product (R_{f}=0.5, 1:1 hexane/EtOAc) were collected and evaporated under vacuum to yield E as a light yellow solid (2.91 g, 11.0 mmol, 46%). ¹H NMR (DMSO-d₆, 300 MHz) δ 4.97 (brs, 2H), 6.21 (d, 1H, J, 9.0), 6.83-6.94 (m, 3H), 8.52 (s, 2H), 9.52 (s, 2H).

### Example 19

### 1-{5-[2-(3-Amino-phenylamino)-pyrimidin-5-yl]-thiophen-2-yl}-ethanone

To a dry microwave tube, E (240 mg, 1.42 mmol), F (340 mg, 1.28 mmol), PdCl₂(dppf)₂ (104 mg, 0.128 mmol), and triethylamine (0.480 mL, 3.46 mmol) were charged in DMF (3 mL). The reaction mixture was purged with N₂ for 10 min then sealed and put into microwave reactor at 120° C. for 20 min. The reaction mixture was cooled down to ambient temperature and evaporated to dryness. The residue was purified using reverse phase Prep HPLC to give product 19 as a light yellow solid (50 mg, 0.16 mmol, 11% yield). ¹H NMR (CDCl₃, 300 MHz) δ 2.57 (s, 3H), 3.73 (brs, 2H), 6.41-6.44 (d, 1H, J=7.8 Hz), 6.86-6.90 (d, 1H, J=8.1 Hz), 7.10-7.24 (t, 1H, J=7.9 Hz), 7.19-7.21 (m, 2H), 7.23-7.24 (, 1H, J=3.9 Hz), 7.66-7.67 (d, 1H, J=3.9 Hz), 8.58 (s, 2H). MS (m/z) 311 [M+1]

### Example 20

### N-(5-Thiophen-2-yl-pyrimidin-2-yl)-benzene-1,3-diamine

To a dry microwave tube, E (240 mg, 1.42 mmol), F (227 mg, 1.28 mmol), PdCl₂(dppf)₂ (104 mg, 0.128 mmol), and triethylamine (0.480 mL, 3.46 mmol) were charged in DMF (3 mL). The reaction mixture was purged with N₂ for 10 min then sealed and put into microwave reactor at 120° C. for 20 min. The reaction mixture was cooled down to ambient temperature and evaporated to dryness. The residue was purified using reverse phase Prep HPLC to give product 19 as an off white solid (13 mg, 0.04 mmol, 14% yield). ¹H NMR (CDCl₃, 300 MHz) δ 3.73 (brs, 2H), 6.40-6.44 (d, 2H), 6.90-6.94 (d, 1H), 7.11-7.46 (m, 6H), 7.82-7.95 (m, 2H), 8.64 (s, 2H). MS (m/z) 319 [M+1]

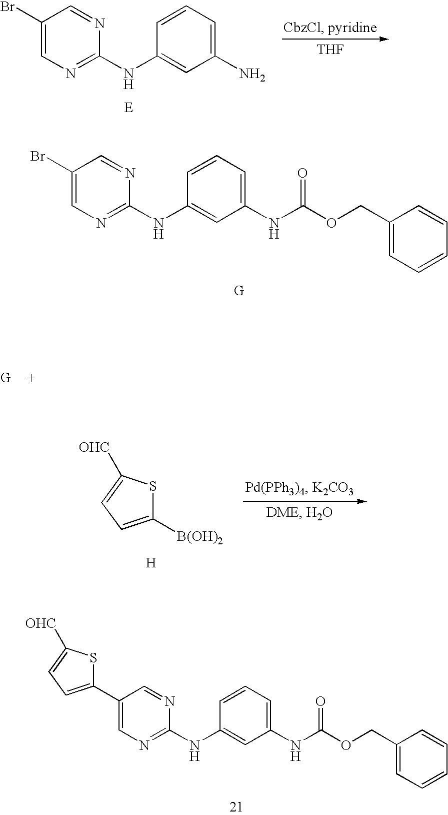

### Benzyl 3-(5-bromopyrimidin-2-ylamino)phenyl carbamate (G)

To a stirred solution of compound E (1.0 g, 3.77 mmol), pyridine (608 μL, 7.54 mmol) in anhydrous THF (20 mL) under N₂ was added benzyl chloroformate (0.64 mL, 4.53 mmol). The reaction mixture was stirred for 0.5 h at ambient temperature. 10 mL of saturated NH₄Cl aqueous solution was added to quench the reaction. The reaction mixture was filtered, the filtrate was evaporated to dryness. The residue was purified using column chromatography (silica gel, 1:1:1 hexane/CH₂Ci2/EtOAc). The fractions containing product (R_{f}=0.5, 1:1 hexane/EtOAc) were collected and evaporated to dryness under vacuum to yield product G as a white solid (1.34 g, 3.4 mmol, 89%). ¹H NMR (CDCl₃, 300 MHz) δ 5.19 (s, 2H), 6.69 (brs, 1H), 7.05-7.08 (m, 1H), 7.18 (brs, 1H), 7.23-7.24 (m, 1H), 7.32-7.42 (m, 6H), 7.82 (m, 1H), 8.42 (s, 2H).

### Example 21

### {3-[5-(5-Formyl-thiophen-2-yl)-pyrimidin-2-ylamino]-phenyl}-carbamic acid benzyl ester

A mixture of G (200 mg, 0.50 mmol), H (118 mg, 0.76 mmol), Pd(PPh₃)₄ (116 mg, 0.10 mmol), and K₂CO₃ (138 mg, 1.00 mmol) in DME (3 mL) and H₂O (1 mL) was charged in a micro wave tube. After purged with N₂ for 10 min, the reaction mixture was sealed and put into microwave reactor at 120° C. for 20 min. The reaction mixture was cool down to room temperature and diluted with EtOAc, then filtered. The filtrate was evaporated to dryness under vacuum. The residue was purified using column chromatography (silica gel, 1:1 hexane/EtOAc). The fractions containing product (R_{f}=0.4, 1:1 hexane/EtOAc) were collected and evaporated to dryness under vacuum to yield product 21 as a pale yellow solid (31 mg, 0.07 mmol, 14%). ¹H NMR (CDCl₃, 300 MHz) δ 5.22 (s, 2H), 6.70 (brs, 1H), 7.05-7.10 (m, 1H), 7.28-7.43 (m, 9H), 7.75-7.76 (d, 1H), 7.93 (brs, 1H), 8.71 (s, 2H), 9.90 (s, 1H). MS (m/z) 431 [M+1].

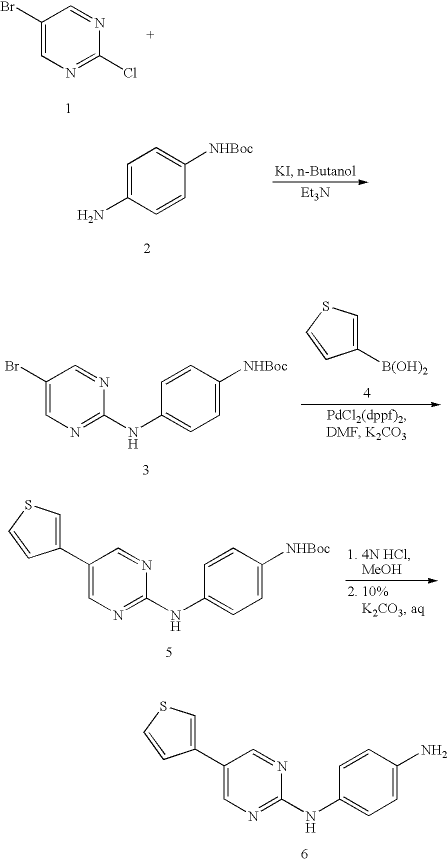

### Preparation of Compound (3)

To a solution of 1 (3.56 g, 18.3 mmol) in n-butanol (40 mL), TEA (5.0 mL, 36.6 mmol), 2 (3.83 g, 18.3 mmol) and KI (3.04 g, 18.36 mmol) were added. Stirred and heated to 90° C. for 21 h, the reaction mixture was cooled to room temperature, and filtered. The filter cake was washed with EtOH and subsequently triturated with a 10% K₂CO₃ aqueous solution (2×5 mL) and water (5 mL). After dried on the vacuum line, compound 3 was yielded as a light yellow solid (3.30 g, 9.0 mmol, 49%). ¹H NMR (CDCl₃, 300 MHz) 1.51 (s, 9H), 6.40 (brs, 1H), 7.01 (brs, 1H), 7.30-7.35 (d, 2H, J, 8.9), 7.44-7.49 (d, 2H, J, 8.9), 8.38 (s, 2H).

### Preparation of Compound (6)

To a dry 10 mL round bottom flask, a mixture of 3 (i83 mg, 0.50 mmol), 4 (96 mg, 0.75 mmol) and Pd (PPh₃)₄ (83 mg, 0.07 mmol) and K₂CO₃ (207 mg, 1.50 mmol) in DMF (2 mL) was bubbled with N₂ for 10 min and was then stirred and heated to 100° C. for 15 hours. The resulting mixture was evaporated to dryness. The crude material containing 5 was dissolved in MeOH (3 mL), and 4N HCl aq solution (5 mL) was added. After stirred and heated at 65° C. for 2.5 h, the reaction mixture was cooled to room temperature. Evaporated the MeOH off, the mixture was filtered. The filtrate was extracted with diethyl ether (2×5 mL) then the aqueous phase was basified with 10% K₂CO₃ aqueous solution to pH 8. Collected precipitate by filtering, compound 6 was obtained as an orange solid (108 mg, 81% pure, 0.33 mmol, 65%). ¹H NMR (CDCl₃, 300 MHz) δ 3.58 (brs, 2H), 6.66-6.73 (d, 2H, J, 11.8), 7.28-7.30 (dd, 2H, J, 5.1, 1.5), 7.31-7.37 (m, 3H), 7.42-7.44 (dd, 1H, J, 5.0, 3.0), 8.59 (s, 2H).

### Examples 22-29

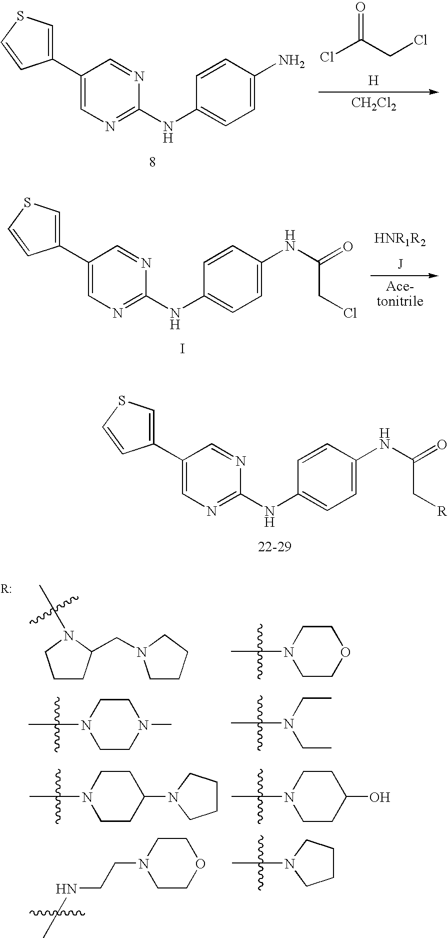

### Compound (I) N-(4-(5-(thiophen-3-yl)pyrimidin-2-ylamino)phenyl)-2-chloroacetamide

To a suspension of 8 (366 mg, 1.36 mmol) in anhydrous CH₂Cl₂ (40 mL), H was added under nitrogen. After stirring at room temperature for 5 min, the reaction mixture was evaporated to dryness. The residue was triturated with CH₂Cl₂ (2 mL) and filtered. The filter cake was triturated added with a saturated Na₂CO₃ aqueous solution (10 mL). After filteration, washed with water (2×2 mL), and dried on the vacuum line, compound I was obtained as a tan solid. ¹H NMR (CDCl₃, 300 MHz) δ 4.13 (s, 2H), 7.14 (brs, 1H), 7.31-7.33 (dd, 1H), 7.40-7.42 (dd, 1H), 7.44-7.46 (dd, 1H), 7.51-7.54 (d, 2H), 7.64-7.66 (d, 2H), 8.19 (brs, 1H), 8.65 (s, 2H).

### Example 22

### 2-(2-Pyrrolidin-1-ylmethyl-pyrrolidin-1-yl)-N-[4-(5-thiophen-3-yl-pyrimidin-2-ylamino)-phenyl]-acetamide

A solution of Compounds (I) (50 mg, 0.15 mmol) and J (142 μL, 0.87 mmol) in acetonitrile (10 mL) was heated to reflux for 13 h under N₂. The resulting mixture was evaporated to dryness. The residue was triturated with 10% K₂CO₃ aqueous solution (5 mL). The mixture was partitioned between EtOAc and water. The combined organic phases was dried (Na₂SO₄), filtered, and evaporated to dryness. The residue was purified using column chromatography (silica gel, 5% MeOH/CH₂Cl₂). The fractions containing product were evaporated to dryness under vacuum to yield 22 as a pale yellow foam. ¹H NMR (CDCl₃, 300 MHz) δ1.5-2.2 (m, 10H), 2.21-3.10 (m, 7H), 3.20 (brs, 2H), 3.49-3.60 (brs, 1H), 7.09 (brs, 1H), 7.30-7.32 (dd, 1H), 7.39-7.41 (dd, 1H), 7.43-7.46 (dd, 1H,), 7.58 (brs, 3H), 8.63 (s, 2H), 9.53 (brs, 1H). MS (m/z) 463 [M+1].

Compounds 23-29 were prepared with a similar procedure.

### Example 23

### 2-(4-methylpiperazin-1-yl)-N-[4-[(5-thien-3-ylpyrimidin-2-yl)amino]phenyl]acetamide

A solution of 1 (50 mg, 0.15 mmol) and J (87 mg, 0.87 mmol) in acetonitrile (10 mL) was heated to reflux for 13 h under N₂ The resulting mixture was evaporated to dryness. The residue was triturated with 10% K₂CO₃ aqueous solution (5 mL. The mixture was partitioned between EtOAc and water. The combined organic phases was dried (Na₂SO₄), filtered, and evaporated to dryness. The residue was purified using column chromatography (silica gel, 5% MeOH/CH₂Cl₂). The fractions containing product were evaporated to dryness under vacuum to yield 23, 74% yield as a light yellow solid. ¹H NMR (CDCl₃, 300 MHz) δ 2.33 (s, 3H), 2.52 (brs, 4H), 2.67 (brs, 4H), 3.14 (s, 2H), 7.11 (brs, 1H), 7.30-7.33 (dd, 1H, J, 4.8, 1.5), 7.40-7.41 (dd, 1H, J, 3.0, 1.5), 7.43-7.46 (dd, 1H, J, 5.1, 3.0), 7.54-7.62 (m, 4H), 8.64 (s, 2H), 9.06 (brs, 1H). MS (m/z) 497 [M+1].

### Example 24

### 2-(4-Pyrrolidin-1-yl-piperidin-1-yl)-N-[4-(5-thiophen-3-yl-pyrimidin-2-ylamino)-phenyl]-acetamide

A solution of 1(50 mg, 0.15 mmol) and J (133 mg, 0.87 mmol) in acetonitrile (10 mL) was heated to reflux for 13 h under N₂. The resulting mixture was evaporated to dryness. The residue was triturated with 10% K₂CO₃ aqueous solution (5 mL. The mixture was partitioned between EtOAc and water. The combined organic phases was dried (Na₂SO₄), filtered, and evaporated to dryness. The residue was purified using column chromatography (silica gel, 5% MeOH/CH₂Cl₂). The fractions containing product were evaporated to dryness under vacuum to yield 24, 89% yield as a off white solid. ¹H NMR (CDCl₃, 300 MHz) δ 1.19 (brs, 2H), 1.68 (brs, 3H), 1.83 (brs, 2H), 1.97-2.01 (d, 2H), 2.27-2.34 (t, 2H), 2.60 (brs, 4H), 2.92-2.96 (d, 2H), 3.10 (s, 2H), 7.10 (brs, 1H), 7.31-7.33 (dd, 1H), 7.40-7.41 (dd, 1H), 7.43-7.46 (dd, 1H), 7.52-7.61 (m, 4H), 8.64 (s, 2H), 9.18 (brs, 1H). MS (m/z) 463 [M+1].

### Example 25

### 2-(2-Morpholin-4-yl-ethylamino)-N-[4-(5-thiophen-3-yl-pyrimidin-2-ylamino)-phenyl]-acetamide

A solution of 1(50 mg, 0.15 mmol) and J (113 mg, 0.87 mmol) in acetonitrile (10 mL) was heated to reflux for 13 h under N₂. The resulting mixture was evaporated to dryness. The residue was triturated with 10% K₂CO₃ aqueous solution (5 mL. The mixture was partitioned between EtOAc and water. The combined organic phases was dried (Na₂SO₄), filtered, and evaporated to dryness. The residue was purified using column chromatography (silica gel, 5% MeOH/CH₂Cl₂). The fractions containing product were evaporated to dryness under vacuum to yield 25, 63% yield as a white solid. ¹H NMR (DMSO-d₆, 300 MHz) δ 2.34-2.41 (m, 7H), 2.62-2.66 (t, 2H), 3.26 (s, 2H), 3.54-3.57 (m, 4H), 7.51-7.54 (d, 2H), 7.59-7.61 (dd, 1H), 7.60-7.71 (m, 3H), 7.89-7.90 (dd, 1H), 8.85 (s, 2H), 9.66 (s, 1H), 9.74 (brs, 1H). MS (m/z) 439 [M+1].

### Example 26

### 2-Morpholin-4-yl-N-[4-(5-thiophen-3-yl-pyrimidin-2-ylamino)-phenyl]-acetamide

A solution of 1(50 mg, 0.15 mmol) and J (100 mg, 0.87 mmol) in acetonitrile (10 mL) was heated to reflux for 13 h under N₂. The resulting mixture was evaporated to dryness. The residue was triturated with 10% K₂CO₃ aqueous solution (5 mL. The mixture was partitioned between EtOAc and water. The combined organic phases was dried (Na₂SO₄), filtered, and evaporated to dryness. The residue was purified using column chromatography (silica gel, 5% MeOH/CH₂Cl₂). The fractions containing product were evaporated to dryness under vacuum to yield 26, 81% yield as a white solid. ¹H NMR (CDCl₃, 300 MHz) δ 2.62-2.65 (m, 4H), 3.15 (s, 2H), 3.77-3.80 (m, 4H), 7.11 (brs, 1H), 7.30-7.33 (dd, 1H), 7.40-7.41 (dd, 1H), 7.43-7.46 (dd, 1H), 7.54-7.63 (m, 4H), 8.64 (s, 2H), 8.99 (brs, 1H).

### Example 27

### 2-Diethylamino-N-[4-(5-thiophen-3-yl-pyrimidin-2-ylamino)-phenyl]-acetamide

A solution of 1(50 mg, 0.15 mmol) and J (100 mg, 0.87 mmol) in acetonitrile (10 mL) was heated to reflux for 13 h under N₂. The resulting mixture was evaporated to dryness. The residue was triturated with 10% K₂CO₃ aqueous solution (5 mL. The mixture was partitioned between EtOAc and water. The combined organic phases was dried (Na₂SO₄), filtered, and evaporated to dryness. The residue was purified using column chromatography (silica gel, 5% MeOH/CH₂Cl₂). The fractions containing product were evaporated to dryness under vacuum to yield 27, 84% yield as a light yellow solid. ¹H NMR (DMSO-d₆, 300 MHz) δ1.00-1.05 (t, 6H), 2.56-2.63 (q, 4H), 3.10 (brs, 2H), 7.45-7.58 (m, 2H), 7.58-7.63 (dd, 1H), 7.66-7.71 (m, 3H), 7.89-7.90 (dd, 1H), 8.85 (s, 2H), 9.49 (s, 1H), 9.66 (s, 1H). MS (m/z) 382 [M+1].

### Example 28

### 2-(4-Hydroxy-piperidin-1-yl)-N-[4-(5-thiophen-3-yl-pyrimidin-2 ylamino)-phenyl]-acetamide

A solution of 1(50 mg, 0.15 mmol) and J (87 mg, 0.87 mmol) in acetonitrile (10 mL) was heated to reflux for 13 h under N₂ The resulting mixture was evaporated to dryness. The residue was triturated with 10% K₂CO₃ aqueous solution (5 mL. The mixture was partitioned between EtOAc and water. The combined organic phases was dried (Na₂SO₄), filtered, and evaporated to dryness. The residue was purified using column chromatography (silica gel, 5% MeOH/CH₂Cl₂). The fractions containing product were evaporated to dryness under vacuum to yield 28, 85% yield as a light yellow solid. ¹H NMR (DMSO-d₆, 300 MHz) δ 1.40-1.60 (m, 2H), 1.70-1.85 (m, 2H), 2.18-2.31 (m, 2H), 2.69-2.85 (m, 2H), 3.05 (s, 2H), 3.40-3.55 (m, 1H), 4.57-4.58 (d, 1H), 7.52-7.58 (m, 2H), 7.60-7.62 (dd, 1H), 7.67-7.71 (m, 3H), 7.90-7.91 (dd, 1H), 8.86 (s, 2H), 9.53 (s, 1H), 9.68 (s, 1H). MS (m/z) 410 [M+1].

### Example 29

### 2-Pyrrolidin-1-yl-N-[4-(5-thiophen-3-yl-pyrimidin-2-ylamino)-phenyl]-acetamide

A solution of 1(50 mg, 0.15 mmol) and J (82 mg, 0.87 mmol) in acetonitrile (10 mL) was heated to reflux for 13 h under N₂ The resulting mixture was evaporated to dryness. The residue was triturated with 10% K₂CO₃ aqueous solution (5 mL. The mixture was partitioned between EtOAc and water. The combined organic phases was dried (Na₂SO₄), filtered, and evaporated to dryness. The residue was purified using column chromatography (silica gel, 5% MeOH/CH₂Cl₂). The fractions containing product were evaporated to dryness under vacuum to yield 29, 73% yield as a light yellow solid. ¹H NMR (DMSO-d₆, 300 MHz) δ 1.72-1.77 (m, 4H), 2.56-2.61 (m, 4H), 3.21 (s, 2H), 7.51-7.57 (m, 2H), 7.59-7.61 (dd, 1H), 7.66-7.70 (m, 3H), 7.89-7.90 (dd, 1H), 8.85 (s, 2H), 9.54 (s, 1H), 9.65 (s, 1H). MS (m/z) 380 [M+1].

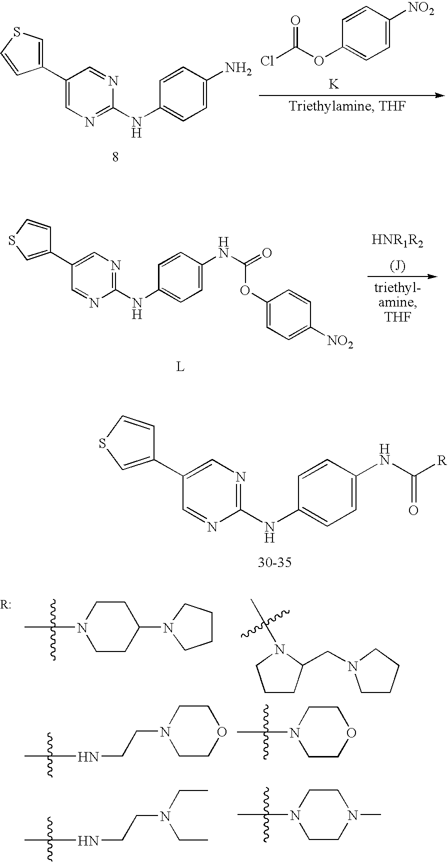

### 4-nitrophenyl 4-(5-(thiophen-3-yl)pyrimidin-2-ylamino)phenylcarbamate (L)

To a solution of 8 (370 mg, 1.38 mmol) in anhydrous THF (10 mL), was added a solution of K (278 mg, 1.38 mmol) in THF (10 mL) dropwise at 0° C. to 5° C. under N₂. After addition, the reaction mixture was allowed to warm up to room temperature and stirred at room temperature for 2 h. The resulting mixture was evaporated to dryness. The residue was triturated with a saturated aqueous NaHCO₃ solution (10 mL). The precipitate was collected by filtering. After drying on the vacuum line, L was obtained as a yellow solid (604 mg, 81% pure, 1.13 mmol, 82%). ¹H NMR (DMSO-d₆, 300 MHz) δ 6.54-6.57 (d, 2H), 7.17-7.20 (d, 2H), 7.52-7.70 (m, 3H), 7.78-7.81 (d, 2H), 7.91-7.93 (dd, 1H), 7.95-7.98 (d, 2H), 8.89 (s, 2H), 9.87 (s, 1H).

### Example 30

### 4-Pyrrolidin-1-yl-piperidine-1-carboxylic acid [4-(5-thiophen-3-yl-pyrimidin-2-ylamino)-phenyl]-amide

A solution of L (81 mg, 0.19 mmol) and J (36 mg, 0.23 mmol), and triethylamine (32 mL, 0.23 mmol) in acetonitrile (10 mL) was stirred at room temperature for 4 h. The resulting mixture was evaporated to dryness. The residue was purified using column chromatography (silica gel, 30% MeOH/CH₂Cl₂). The fractions containing product were evaporated to dryness under vacuum to yield 30 (73 mg, 0.16 mmol, 87%) as a pale yellow solid. ¹H NMR (DMSO-d₆, 300 MHz) δ 0.92-0.97 (t, 1H, J, 6.9), 1.20-1.45 (m, 2H), 1.61-1.73 (m, 4H), 1.79-1.90 (m, 2H), 2.17 (brs, 1H), 2.82-2.90 (t, 2H, J, 11.1), 3.97-4.02 (d, 2H, J, 13.2), 7.32-7.35 (d, 2H, J, 9.0), 7.58-7.62 (m, 3H), 7.66-7.69 (dd, 1H, J, 4.8, 3.0), 7.87-7.89 (dd, 1H, J, 3.0, 1.3), 8.35 (brs, 1H), 8.83 (s, 2H), 9.55 (s, 1H). MS (m/z) 449 [M+1].

Compounds 31-35 were also prepared with a similar procedure.

### Example 31

### 1-(2-Morpholin-4-yl-ethyl)-3-[4-(5-thiophen-3-yl-pyrimidin-2-ylamino)-phenyl]-urea

A solution of L (81 mg, 0.19 mmol) and J (29 mg, 0.23 mmol), and triethylamine (32 μL, 0.23 mmol) in acetonitrile (10 mL) was stirred at room temperature for 4 h. The resulting mixture was evaporated to dryness. The residue was purified using column chromatography (silica gel, 30% MeOH/CH₂Cl₂). The fractions containing product were evaporated to dryness under vacuum to yield 31, 87% yield as a white solid. ¹H NMR (DMSO-d₆, 300 MHz) δ 2.35-2.39 (m, 6H), 3.17-3.23 (m, 2H), 3.57-3.60 (m, 4H), 5.97-6.01 (t, 1H), 7.28-7.31 (d, 2H), 7.57-7.65 (m, 3H), 7.58-7.61 (dd, 1H), 7.87-7.89 (dd, 1H), 8.47 (s, 1H), 8.82 (s, 2H), 9.54 (s, 1H). MS (m/z) 425 [M+1].

### Example 32

### 1-(2-Diethylamino-ethyl)-3-[4-(5-thiorhen-3-yl-pyrimidin-2-ylamino)-phenyl]-urea

A solution of L (81 mg, 0.19 mmol) and J (26 mg, 0.23 mmol), and triethylamine (32 μL, 0.23 mmol) in acetonitrile (10 mL) was stirred at room temperature for 4 h. The resulting mixture was evaporated to dryness. The residue was purified using column chromatography (silica gel, 30% MeOH/CH₂Cl₂). The fractions containing product were evaporated to dryness under vacuum to yield 32, 56% yield as a white solid. ¹H NMR (DMSO-d₆, 300 MHz) δ 0.97-0.99 (t, 6H), 2.43-2.52 (m, 6H), 3.10-3.14 (m, 2H), 5.92-5.96 (m, 1H), 7.27-7.30 (d, 2H), 7.55-7.65 (m, 3H), 7.66-7.68 (dd, 1H), 7.87-7.88 (dd, 1H), 8.51 (brs, 1H), 8.82 (s, 2H), 9.52 (s, 1H). MS (m/z) 411 [M+1].

### Example 33

### 2-Pyrrolidin-1-ylmethyl-pyrrolidine-1-carboxylic acid [4-(5-thiophen-3-yl-pyrimidin-2-ylamino)-phenyl]-amide

A solution of L (81 mg, 0.19 mmol) and J (35 mg, 0.23 mmol), and triethylamine (32 μL, 0.23 mmol) in acetonitrile (10 mL) was stirred at room temperature for 4 h. The resulting mixture was evaporated to dryness. The residue was purified using column chromatography (silica gel, 30% MeOH/CH₂Cl₂). The fractions containing product were evaporated to dryness under vacuum to yield 33, 92% yield as a light yellow solid. ¹H NMR (DMSO-d₆, 300 MHz) δ 1.60-1.85 (m, 7H), 1.95-2.05 (m, 1H), 2.45-2.85 (m, 6H), 3.20-3.30 (m, 1H), 3.50-3.62 (m, 1H), 3.90-4.04 (brs, 1H), 7.24-7.27 (d, 2H), 7.55-7.65 (m, 3H), 7.66-7.69 (dd, 1H), 7.88-7.89 (dd, 1H), 8.83 (s, 2H), 9.56 (s, 1H). MS (m/z) 449 [M+1].

### Example 34

### Morpholine-4-carboxylic acid [4-(5-thiophen-3-yl-pyrimidin-2-ylamino)-phenyl]-amide

A solution of L (81 mg, 0.19 mmol) and J (20 mg, 0.23 mmol), and triethylamine (32 μL, 0.23 mmol) in acetonitrile (10 mL) was stirred at room temperature for 4 h. The resulting mixture was evaporated to dryness. The residue was purified using column chromatography (silica gel, 30% MeOH/CH₂Cl₂). The fractions containing product were evaporated to dryness under vacuum to yield 34, 99% yield as a white solid. ¹H NMR (DMSO-d₆, 300 MHz) δ 3.39-3.42 (m, 4H), 3.58-3.62 (m, 4H), 7.33-7.36 (d, 2H), 7.59-7.67 (m, 3H), 7.66-7.69 (dd, 1H), 7.88-7.89 (dd, 1H), 8.41 (brs, 1H), 8.84 (s, 2H). MS (m/z) 382 [M+1].

### Example 35

### 4-Methyl-piperazine-1-carboxylic acid [4-(5-thiophen-3-yl-pyrimidin-2-ylamino)-phenyl]-amide

A solution of L (81 mg, 0.19 mmol) and J (20 mg, 0.23 mmol), and triethylamine (32 μL, 0.23 mmol) in acetonitrile (10 mL) was stirred at room temperature for 4 h. The resulting mixture was evaporated to dryness. The residue was purified using column chromatography (silica gel, 30% MeOH/CH₂Cl₂). The fractions containing product were evaporated to dryness under vacuum to yield 35, 77% yield as a yellow solid. ¹H NMR (DMSO-d₆, 300 MHz) δ 2.08 (s, 3H), 2.29-2.32 (m, 4H), 3.40-3.43 (m, 4H), 7.33-7.36 (d, 2H), 7.59-7.63 (m, 3H), 7.66-7.69 (dd, 1H), 7.88-7.89 (dd, 1H), 8.37 (brs, 1H), 8.84 (s, 2H), 9.58 (s, 1H). MS (m/z) 395 [M+1].

### Example 36

### N-[3-(5-Thiophen-3-yl-pyrimidin-2-ylamino)-phenyl]-acetamide

A 25-mL, one-neck, round bottom flask equipped with a magnetic stirrer was charged with E (50 mg, 0.93 mmol), acetic anhydride (104 mg, 1.02 mmol), pyridine (162 mg, 2.05 mmol) and anhydrous THF (5.0 mL). After stirring at ambient temperature for 2 hours the reaction was quenched with a 25% aqueous ammonium chloride solution (5 mL) and extracted with ethyl acetate (30 mL). The organic layer was dried over sodium sulfate and concentrated under reduced pressure. Trituration with 1:1:2 ethyl acetate/hexanes/methylene chloride mixture followed by filtration afforded a 75% yield (217 mg) of 36 as an off-white solid. ¹HNMR (400 MHz, DMSO-d₆) δ 8.76 (s, 2H), 8.10 (s, 1H), 7.69 (s, 1H), 7.57 (m, 1H), 7.47 (m, 1H), 7.39 (m, 1H), 7.24 (m, 2H), 2.14 (s, 3H). MS m/z 311 [M⁺+1].

### Example 37

### N-Isopropyl-N′-(5-thiophen-3-yl-pyrimidin-2-yl)-benzene-1,3-diamine

A 25-mL, three-neck, round bottom flask equipped with a magnetic stirrer was charged with E (250 mg, 0.93 mmol), 3 Å molecular sieves (250 mg), acetone (54 mg, 0.93 mmol), and anhydrous THF (1.5 mL). After stirring at ambient temperature for 2 hours, sodium triacetoxyborohydride (237 mg, 1.12 mmol) was added and the reaction stirred for an additional 18 hours. The reaction was quenched with a solution of saturated sodium bicarbonate (3.0 mL) and extracted with ethyl acetate (30 mL). The organic layer was dried over sodium sulfate, filtered and concentrated under reduced pressure. Trituration of the residue with methanol (2.0 mL) followed by filtration afforded a 22% yield (65 mg) of 37 as an off-white solid. ¹HNMR (400 MHz, DMSO-d₆) δ 8.69 (s, 2H), 7.48 (m, 1H), 7.44 (m, 1H), 7.34 (d, 1H), 7.16 (multiplets, 2H), 6.87 (d, 1H), 6.33 (d, 1H), 3.69 (q, 1H), 1.09 (d, 6H). MS m/z 311 [M⁺+1].

### Example 38

### N,N-Diethyl-N-(5-thiophen-3-yl-pyrimidin-2-yl)-benzene-1,3-diamine

A 25-mL, three-neck, round bottom flask equipped with a magnetic stirrer was charged with E (250 mg, 0.93 mmol), 3 Å molecular sieves (250 mg), acetaldehyde (103 mg, 2.33 mmol), and anhydrous THF (1.5 mL). After stirring at ambient temperature for 2 hours, sodium triacetoxyborohydride (493 mg, 2.33 mmol) was added and the reaction stirred for an additional 18 hours. The reaction was then quenched with a solution of saturated sodium bicarbonate (3.0 mL) and extracted with ethyl acetate (30 mL). The organic layer was dried over sodium sulfate, filtered and concentrated under reduced pressure. Trituration of the residue with hexanes (2.0 mL) followed by filtration afforded a 43% yield (129 mg) of 38 as yellow solid. ¹HNMR (400 MHz, DMSO-d₆) δ 8.68 (s, 2H), 7.44 (m, 2H), 7.30 (m, 2H), 7.13 (multiplets, 2H), 6.86 (d, 1H), 6.43 (d, 1H), 3.40 (q, 4H), 1.20 (t, 6H). MS m/z 325 [M⁺+1]. Yield 43%.

### Example 39

### (3-Morpholin-4-yl-phenyl)-(5-thiophen-3-yl-pyrimidin-2-yl)-amine

A 5-mL reaction vial equipped with a magnetic stirrer was charged with 3-Iodo-phenylamine (438 mg, 2.00 mmol), morpholine (348 mg, 4.00 mmol), copper iodide (38 mg, 0.20 mmol), potassium phosphate (850 mg, 4.0 mmol), ethylene glycol (248 mg, 4.00 mmol), and isopropanol (2.0 mL). After stirring at 90° C. for 18 h the reaction was concentrated under reduced pressure, dissolved in methylene chloride (3.0 mL) and washed with water (3.0 mL). The organic layer was then dried over sodium sulfate, filtered, concentrated under reduced pressure and the residue purified by column chromatography (methanol/methylene chloride) affording a 48% yield (172 mg) of 3-morpholin-4-yl-phenylamine as a colorless oil.

A 25-mL, pear shaped flask equipped with a magnetic stirrer and reflux condenser was charged with 3-Morpholin-4-yl-phenylamine (175 mg, 0.97 mmol), 2-Fluoro-5-thiophen-3-yl-pyrimidine (172 mg, 0.97 mmol), triethylamine (98 mg, 0.97 mmol), and 1-butanol (2.0 mL). After heating at 100° C. for 18 h the reaction was concentrated under reduced pressure and the residue purified by column chromatography (hexanes/ethyl acetate) affording 39 in a 46% yield (152 mg) as a white solid. ¹HNMR (400 MHz, DMSO-d₆) δ 9.59 (s, 1H), 8.92 (s, 2H), 7.94 (m, 1H), 7.70 (m, 1H), 7.63 (d, 1H), 7.45 (m, 1H), 7.27 (m, 1H), 7.13 (m, 1H), 6.54 (m, 1H), 3.76 (m, 4H), 3.09 (m, 4H). MS m/z 339 [M⁺+1].

### Example 40

### 4-(4-Methyl-piperazin-1-ylmethyl)-N-[2-methyl-5-(5-thiophen-3-yl-pyrimidin-2-ylamino)-phenyl]-benzamide

6-Methyl-3-(5-thiophen-3-yl-pyrimidin-2-yl-amino aniline: A 50-mL, three-neck, round bottom flask equipped with a magnetic stirrer and reflux condenser was charged with 2-Fluoro-5-thiophen-3-yl-pyrimidine (500 mg, 2.77 mmol), 4-Methyl-benzene-1,3-diamine (1.01 g, 8.32 mmol) and isopropanol (10 mL). After stirring for 18 hours at 90° C. the reaction was concentrated under reduced pressure and the residue purified by column chromatography (hexane/ethyl acetate) affording a 50% yield of 6-Methyl-3-(5-thiophen-3-yl-pyrimidin-2-yl-amino aniline as a white solid.

A 50-mL, three-neck, round bottomed flask equipped with a magnetic stirrer was charged with 4-Methyl-N1-(5-thiophen-3-yl-pyrimidin-2-yl)-benzene-1,3-diamine (371 mg, 1.31 mmol), 4-(4-Methyl-piperazin-1-ylmethyl)-benzoic acid (402 mg, 1.31 mmol), N,N-diisopropylethylamine (171 mg, 1.57 mmol), and anhydrous DMF (3.0 mL). To the resulting mixture were added 1-(3-dimethylaminopropyl)-3-ethylcarbodiimide hydrochloride (302 mg, 1.57 mmol) and 1-hydroxy-7-azabenzotriazole (89 mg, 0.655 mmol). After stirring for 20 h at ambient temperature, the reaction mixture was evaporated to dryness, purified by column chromatography (methanol/methylene chloride), and then triturated with acetonitrile, filtered and the filter cake dried under vacuum affording an 72% yield of Example 40 as a white solid. ¹HNMR (400 MHz, DMSO-d₆) δ 8.76 (s, 2H), 7.99 (d, 2H), 7.85 (s, 1H), 7.66 (s, 1H), 7.53 (m, 4H), 7.43 (d, 1H), 7.23 (d, 1H), 3.70 (m, 2H), 2.93 (m, 4H), 2.69 (m, 4H), 2.58 (s, 3H), 2.29 (s, 3H). MS m/z 499 [M⁺+1].

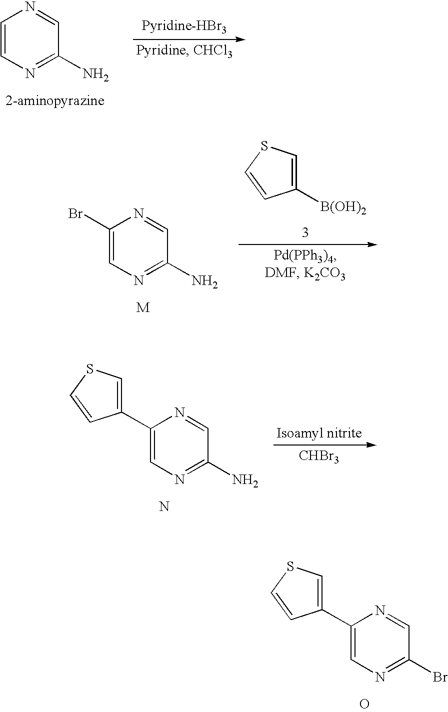

### 5-bromopyrazin-2-amine (M)

To a solution of 2-aminopyrazine (1.00 g, 10.0 mmol) in chloroform (50 mL) and pyridine (0.8 mL, 10.0 mmol), solid pyridine-HBr₃ (3.37 g, 10.0 mmol) was added in portions over 10 min. The reaction mixture was stirred at room temperature for 2 h. Saturated NaHCO₃ aqueous solution (25 mL) was added to this reaction mixture carefully (pH 7 to 8) and stirred for 10 min. Organic layer was separated, washed with water (15 mL×3) (filtered if necessary), dried with Na₂SO₄, filtered, and evaporated to dryness. The residue was purified using column chromatography (silica gel, 1:1:8 hexane/CH₂Cl₂/EtOAc). The fractions containing product were evaporated to dryness under vacuum to yield compound M as a pale yellow solid (601 mg, 3.45 mmol, 35%). ¹H NMR (DMSO-d₆, 300 MHz) δ 6.63 (bs, 2H), 7.67 (d, 1H, J, 1.4), 8.02 (d, 1H, J, 1.4).

### 5-(thiorhen-3-yl)pyrazin-2-amine (N)

In a dry 100 mL round bottom flask, a mixture of M (2.30 g, 13 mmol), thiophene-3-boronic acid (2.54 g, 20.0 mmol), Pd(PPhS)₄ (2.29 g, 2.0 mmol) and K₂CO₃ (5.47 g, 40.0 mmol) in DMF (40 mL) was bubbled with N₂ for 10 min, then the mixture was stirred at 100° C. for 16 hours. The resulting mixture was evaporated to dryness. The residue was purified using column chromatography (silica gel, 2:1:7 hexane/CH₂Cl₂/EtOAc). The fractions containing product were evaporated to dryness under vacuum to yield compound N as a light brown solid (1.63 g, 9.22 mmol, 71%). ¹H NMR (DMSO-d₆, 300 MHz) δ 6.46 (bs, 2H), 7.55-7.65 (m, 2H), 7.85 (bs, 1H), 7.90 (d, 1H, J, 1.4), 8.42 (d, 1H, J, 1.4).

### 2-bromo-5-(thiophen-3-yl)pyrazine (O)

To a hot (95° C. to 100° C.) solution of N (1.63 g, 9.22 mmol) in bromoform (150 mL), isoamyl nitrite (3.22 mL, 24.0 mmol) was added in one portion under nitrogen. After the reaction mixture was stirred and refluxed for 4 h, another portion of isoamyl nitrite (3.22 mL, 24.0 mmol) was added then continued to reflux over night. After evaporated to dryness, the residue of the reaction mixture was purified using column chromatography (silica gel, 6:1 hexane/MTBE). The fractions containing product were evaporated to dryness under vacuum to yield compound O as a light yellow solid (1.40 g, 5.8 mmol, 63%). ¹H NMR (CDCl₃, 300 MHz) δ 7.45 (dd, 1H, J, 6.0, 3.0), 7.64 (dd, 1H, J, 5.1, 1.3), 7.99 (dd, 1H, J, 3.0, 1.3), 8.65 (d, 1H, J, 1.4), 8.66 (d, 1H, J, 1.4).

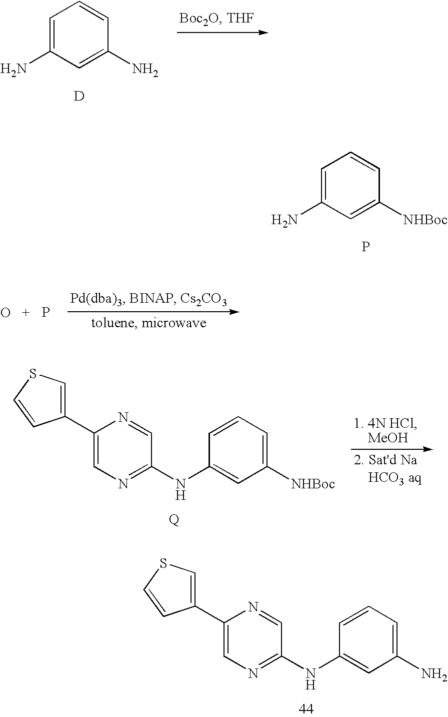

### tert-butyl 3-aminophenylcarbamate (P)

To a solution of D (3.24 g, 30.0 mmol) in THF (20 mL), Boc₂O (2.18 g, 10.0 mmol) was added under N₂. The reaction mixture was stirred at room temperature for 24 h. MTBE (50 mL) was added to dilute, then the mixture was washed with water (20 mL×3). Organic layer was combined and dried with Na₂SO₄, filtered, and evaporated to dryness. The residue was purified using column chromatography (silica gel, 1:1 hexane/EtOAc). The fractions containing product were evaporated to dryness under vacuum to yield compound P as a white solid (1.92 g, 9.2 mmol, 92%). ¹H NMR (CDCl₃, 300 MHz) δ 1.50 (s, 9H), 3.65 (bs, 2H), 6.35 (dd, 1H, J, 7.8, 3.0), 6.35 (bs, 1H), 6.53 (dd, 1H, J, 8.1, 2.0), 6.97 (m, 1H), 7.03 (t, 1H, J, 8.4).

### N-(5-Thiophen-3-yl-pyrazin-2-yl)-benzene-1,3-diamine

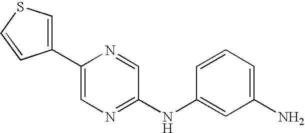

To a dry microwave tube, a mixture of 0 (136 mg, 0.56 mmol), P (125 mg, 0.60 mmol), Pd₂(dba)₃ (46 mg, 0.05 mmol), BINAP (47 mg, 0.075 mmol), and Cs₂CO₃ (228 mg, 0.70 mmol) in toluene (1 mL) was charged and bubbled with N₂ for 10 min. This tube was put into microwave reactor at 120° C. for 1 h. After evaporated to dryness, the residue was purified using column chromatography (silica gel, 5:3:2 hexane/MTBE/CH₂Cl₂). The fractions containing product were evaporated to dryness under vacuum to yield compound Q as a brown foam. This brown foam was dissolved in methanol (20 mL) and a 4N HCl (20 mL) aqueous solution was added. After stirred at 65° C. for 1 h, the reaction mixture was evaporated and basified to pH 8 with saturated NaHCO₃ aqueous solution. The precipitate was collected by filtration. After washed with water and dried on the vacuum line, compound 44 was provided as a dark brown solid (25 mg, 0.09 mmol, 17%). ¹H NMR (CDCl₃, 300 MHz) δ 3.75 (bs, 2H), 6.42 (d, 1H, J, 7.8), 6.51 (bs, 1H), 6.73 (d, 1H, J, 7.2), 6.89 (s, 1H), 7.13 (t, 1H, J, 7.8), 7.41 (dd, 1H, J, 4.8, 3.0), 7.57 (d, 1H, J, 6.0), 7.75 (d, 1H, J, 3.0), 8.28 (s, 1H), 8.47 (s, 1H).

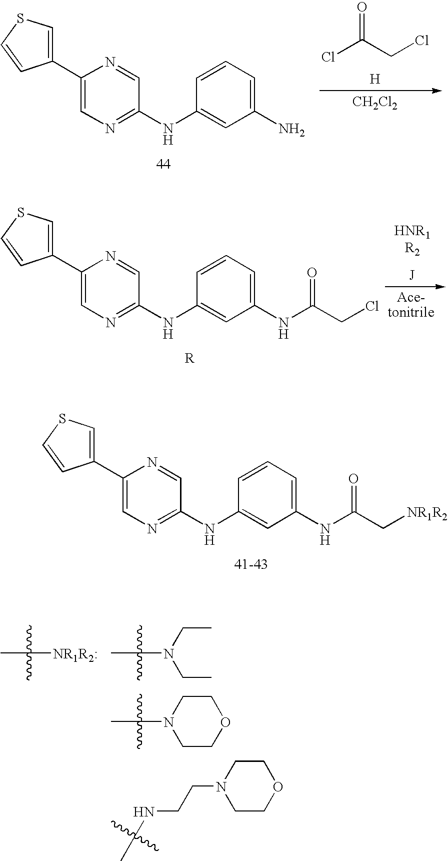

### Preparation of Compound (R)

To a suspension of 44 (40 mg, 0.15 mmol) in anhydrous CH₂Cl₂ (4 mL), H (13 μL, 016 mmol) was added under nitrogen. After stirred at room temperature for 10 min, saturated NaHCO₃ (4 mL) was added and stirred for 10 min. Organic layer was separated, dried with Na₂SO₄, filtered, and evaporated to dryness. The residue was purified using column chromatography (silica gel, 1:1 hexane/EtOAc). The fractions containing product were evaporated to dryness under vacuum to yield compound R as a yellow solid. ¹H NMR (CDCl₃, 300 MHz) δ 4.12 (s, 1H), 4.20 (s, 2H), 6.62 (s, 1H), 7.15-7.19 (m, 1H), 7.30-7.31 (m, 1H), 7.41 (dd, 1H, J, 5.1, 3.0), 7.60 (dd, 1H, J, 5.4, 1.2), 7.78 (dd, 1H, J, 3.0, 1.2), 7.94 (m, 1H), 8.24 (bs, 1H), 8.26 (d, 1H, J, 1.5), 8.52 (d, 1H, J, 1.5).

### Example 41

### 2-Diethylamino-N-[3-(5-thiophen-3-yl-pyrazin-2-ylamino)-phenyl]-acetamide

A solution of R (57 mg, 0.17 mmol) and J (diethyl amine 124 μL) in acetonitrile (10 mL) was heated to reflux for 4.5 h under N₂. The resulting mixture was evaporated to dryness. The residue was purified using column chromatography (silica gel, 3% MeOH/CH₂Cl₂). The fractions containing product were evaporated to dryness under vacuum to yield compound 45 as a pale yellow solid (62 mg, 0.16 mmol, 95%). ¹H NMR (CDCl₃, 300 MHz) δ 1.10 (t, 6H, J, 7.2), 2.66 (q, 4H, J, 7.2), 3.15 (s, 2H), 6.62 (bs, 1H), 7.09-7.13 (m, 1H), 7.29-7.31 (m, 2H), 7.40 (dd, 1H, J, 5.0, 3.0), 7.59 (dd, 1H, J, 5.1, 1.2), 7.76 (dd, 1H, J, 3.0, 1.2), 7.90 (bs, 1H), 8.28 (d, 1H, J, 1.4), 8.51 (d, 1H, J, 1.4), 9.38 (bs, 1H. ¹HNMR (400 MHz, DMSO-d₆) δ 9.48 (s, 1H), 8.53 (s, 1H), 8.30 (s, 1H), 7.93 (s, 1H), 7.60 (d, 1H), 7.43 (m, 1H), 7.31 (m, 2H), 6.64 (m, 1H), 3.20 (s, 2H), 2.66 (q, 4H), 1.15 (t, 6H). MS m/z 382 [M⁺+1]. Yield 81%.

### Example 42

### 2-Morpholin-4-yl-N-[3-(5-thiophen-3-yl-pyrazin-2-ylamino)-phenyl]-acetamide

A solution of R (57 mg, 0.17 mmol) and J (104 mg, 1.2 mmol) in acetonitrile (10 mL) was heated to reflux for 4.5 h under N₂. The resulting mixture was evaporated to dryness. The residue was purified using column chromatography (silica gel, 3% MeOH/CH₂Cl₂). The fractions containing product were evaporated to dryness under vacuum to yield compound 42, 81% yield as a light yellow solid. ¹H NMR (CDCl₃, 300 MHz) δ 2.62-2.65 (m, 4H), 3.15 (s, 2H), 3.77-3.80 (m, 4H), 6.59 (bs, 1H), 7.09-7.13 (m, 1H), 7.28-7.32 (m, 2H), 7.41 (dd, 1H, J, 5.1, 3.0), 7.59 (dd, 1H, J, 5.1, 1.2), 7.77 (dd, 1H, J, 3.0, 1.2), 7.92 (s, 1H), 8.27 (d, 1H, J, 1.3), 8.51 (d, 1H, J, 1.3), 9.08 (bs, 1H). ¹HNMR (400 MHz, DMSO-d₆) δ 9.10 (br s, 1H, NH), 8.54 (s, 1H), 8.32 (s, 1H), 7.95 (m, 1H), 7.81 (m, 1H), 7.62 (d, 1H), 7.45 (d, 1H), 7.32 (m, 2H), 7.10 (m, 1H), 6.60 (br s, 1H, NH), 3.82 (m, 4H), 3.18 (s, 2H), 2.67 (m, 4H). MS m/z 396 [M⁺+1]. Yield 83

### Example 43

### 2-(2-Morpholin-4-yl-ethylamino)-N-[3-(5-thiophen-3-yl-pyrazin-2-ylamino)-phenyl]-acetamide

A solution of R (57 mg, 0.17 mmol) and J (156 mg, 1.2 mmol) in acetonitrile (10 mL) was heated to reflux for 4.5 h under N₂. The resulting mixture was evaporated to dryness. The residue was purified using column chromatography (silica gel, 3% MeOH/CH₂Cl₂). The fractions containing product were evaporated to dryness under vacuum to yield compound 43 34% yield as a light yellow foam. ¹H NMR (CDCl₃, 300 MHz) δ 2.45-2.50 (m, 4H), 2.50-2.53 (m, 2H), 2.76-2.80 (m, 2H), 3.40 (s, 2H), 3.70-3.73 (m, 4H), 6.62 (s, 1H), 7.14-7.19 (m, 1H), 7.27-7.32 (m, 2H), 7.41 (dd, 1H, J, 4.8, 3.0), 7.58 (dd, 1H, J, 4.8, 1.2), 7.77 (dd, 1H, J, 3.0, 1.2), 7.94-7.95 (m, 1H), 8.27 (d, 1H, J, 1.4), 8.51 (d, 1H, J, 1.4), 9.43 (bs, 1H).

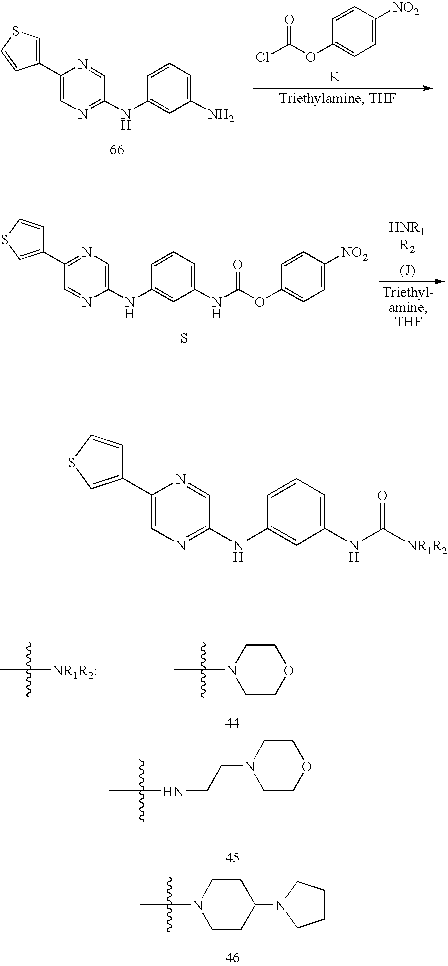

### 4-nitrophenyl 3-(5-(thiophen-3-yl)pyrazin-2-ylamino)phenylcarbamate (Compound S)

To a solution of K (96 mg, 0.46 mmol) and triethylamine (64 μL, 0.46 mmol) in anhydrous THF (4 mL), was added a solution of 66 (123 mg, 0.46 mmol) in THF (5 mL) dropwise at 0° C. to 5° C. under N₂. After addition, the reaction mixture was allowed to warm up to room temperature and stirred at room temperature for 2 h. The resulting mixture was evaporated to dryness. The residue was purified using column chromatography (silica gel, 6:4 hexane/EtOAc). The fractions containing product were evaporated to dryness under vacuum to yield compound S as a yellow solid (84 mg, 0.19 mmol, 42%). ¹H NMR (CDCl₃, 300 MHz) δ 3.97 (bs, 1H), 6.60 (bs, 1H), 7.01 (d, 1H, J, 8.4), 7.21 (d, 1H, J, 8.5), 7.32-7.36 (m, 1H), 7.41 (d, 2H, J, 9.0), 7.42 (dd, 1H, J, 5.1, 3.0), 7.59 (dd, 1H, J, 4.8, 1.3), 7.78 (dd, 1H, J, 3.0, 1.3), 7.84-7.87 (m, 1H), 8.27 (d, 1H, J, 1.4), 8.30 (d, 2H, J, 9.0), 8.51 (d, 1H, J, 1.4).

### Example 44

### Morpholine-4-carboxylic acid [3-(5-thiophen-3-yl-pyrazin-2-ylamino)-phenyl]-amide

To a solution of S (28 mg, 0.065 mmol) and J (7 μL, 0.078 mmol) in THF (2 mL), triethylamine (11 μL, 0.078 mmol) was added. After stirring at room temperature for 15 h, the resulting mixture was evaporated to dryness. The residue was purified using column chromatography (silica gel, 5% MeOH in 1:1 hexane/EtOAc). The fractions containing product were evaporated to dryness under vacuum to yield compound 48 (19 mg, 0.049 mmol, 75%) as a pale yellow solid. ¹H NMR (CDCl₃, 300 MHz) δ 3.48-3.51 (m, 4H), 3.74-3.77 (m, 4H), 6.33 (s, 1H), 6.57 (s, 1H), 6.98 (d, 1H, J, 9.0), 7.14 (d, 1H, J, 9.0), 7.41 (dd, 1H, J, 4.8, 3.0), 7.58 (dd, 1H, J, 4.8, 1.2), 7.66-7.70 (m, 1H), 7.76 (dd, 1H, J, 3.0, 1.2), 8.27 (d, 1H, J, 1.4), 8.49 (d, 1H, J, 1.4). MS m/z 382 (M⁺+1).

### Example 45

### 1-(2-Morpholin-4-yl-ethyl)-3-[3-(5-thiophen-3-yl-pyrazin-2-ylamino)-phenyl]-urea

To a solution of S (28 mg, 0.065 mmol) and J (10 μL, 0.078 mmol) in THF (2 mL), triethylamine (11 μL, 0.078 mmol) was added. After stirring at room temperature for 15 h, the resulting mixture was evaporated to dryness. The residue was purified using column chromatography (silica gel, 5% MeOH in 1:1 hexane/EtOAc). The fractions containing product were evaporated to dryness under vacuum to yield compound 45 in 75% yield as a yellow foam. ¹H NMR (DMSO-d₆, 300 MHz) δ 2.37-2.41 (m, 6H), 3.18-3.24 (m, 2H), 3.58-3.61 (m, 4H), 6.07 (t, 1H, J, 5.4), 7.00 (d, 1H, J, 8.1), 7.13 (t, 1H, J, 7.8), 7.31 (d, 1H, J, 8.1), 7.64 (dd, 1H, J, 5.0, 3.0), 7.68 (dd, 1H, J, 4.8, 1.3), 7.77-7.78 (m, 1H), 7.98 (dd, 1H, J, 3.0, 1.3), 8.26 (d, 1H, J, 1.3), 8.59 (s, 1H), 8.62 (d, 1H, J, 1.3), 9.50 (s, 1H). MS m/z 425 (M⁺+1). ¹HNMR (400 MHz, DMSO-d₆) δ 9.53 (s, 1H), 8.64 (d, 2H), 8.30 (s, 1H), 8.03 (s, 1H), 7.80 (s, 1H), 7.70 (m, 1H), 7.63 (m, 1H), 7.34 (d, 1H), 7.15 (t, 1H), 7.01 (d, 1H), 6.11 (brt, 1H, NH), 3.62 (m, 4H), 3.24 (m, 2H), 2.40 (m, 6H). MS m/z 425 [M⁺+1].

Yield 75%.

### Example 46

### 4-Pyrrolidin-1-yl-piperidine-1-carboxylic acid [3-(5-thiophen-3-yl-pyrazin-2-ylamino)-phenyl]-amide

To a solution of S (28 mg, 0.065 mmol) and J (12 μL, 0.078 mmol) in THF (2 mL), triethylamine (11 μL, 0.078 mmol) was added. After stirring at room temperature for 15 h, the resulting mixture was evaporated to dryness. The residue was purified using column chromatography (silica gel, 5% MeOH in 1:1 hexane/EtOAc). The fractions containing product were evaporated to dryness under vacuum to yield compound 46 in 65% yield as a light yellow foam. ¹H NMR (CDCl₃, 300 MHz) δ1.70-1.90 (m, 5H), 1.93-1.98(m, 2H), 2.19-2.24 (m, 1H), 2.51-2.65 (m, 5H), 2.95-3.05 (m, 2H), 3.99-4.03 (m, 2H), 6.38 (s, 1H), 6.59 (s, 1H), 6.96 (d, 1H, J, 7.8), 7.13 (d, 1H, J, 7.2), 7.22 (s, 1H), 7.40 (dd, 1H, J, 7.8, 3.0), 7.58 (dd, 1H, J, 5.1, 1.2), 7.64-7.66 (m, 1H), 7.76 (dd, 1H, J, 3.0, 1.2), 8.26 (d, 1H, J, 1.4), 8.49 (d, 1H, J, 1.4). MS m/z 449 (M⁺+1). ¹HNMR (400 MHz, DMSO-d₆) δ 8.51 (s, 1H), 8.30 (s, 1H), 7.76 (s, 1H), 7.67 (s, 1H), 7.59 (m, 1H), 7.43 (m, 1H), 7.23 (m, 1H), 7.15 (d, 1H), 6.97 (d, 1H), 6.51 (br s, 1H, NH), 6.39 (br s, 1H, NH), 4.00 (m, 2H), 3.01 (t, 2H), 2.61 (m, 4H), 2.24 (m, 1H), 1.97 (m, 2H), 1.84 (m, 4H), 1.59 (m, 2H). MS m/z 449 [M⁺+1]. Yield 65%.

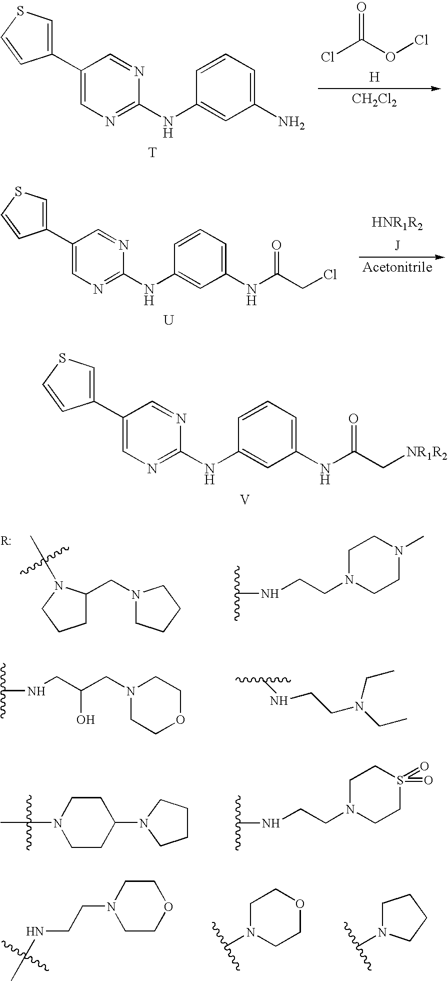

### 2-Chloro-N-[3-(5-thiophen-3-yl-pyrimidin-2-ylamino)-phonyl]-acetamide (U)

To a solution of N-(5-Thiophen-3-yl-pyrimidin-2-yl)-benzene-1,3-diamine (100 mg, 0.37 mmol) in CH₂Cl₂ was added H (42 mg, 0.37 mmol) via micropipette. TLC analysis showed that all starting material had been consumed at which point the solvent was removed in vacuo to provide a yellow solid which was taken up in ethyl acetate (200 mL) and washed with 2 N Na₂CO₃ (2×50 mL). The organics were removed in vacuo and the residue purified via column chromatography (silica gel, 1:3 hexane/EtOAc) The fractions containing product were evaporated to dryness under vacuum to yield compound U in 88% yield as a white crystalline solid. ¹H NMR (300 MHz, CDCl₃) δ 3.5 (s, 1H), 4.15 (s, 2H), 7.1-7.4 (m, 6H), 8.0 (d, 1H), 8.21 (s, 1H), 8.77 (d, 2H). MS m/z 345 [M⁺+1].

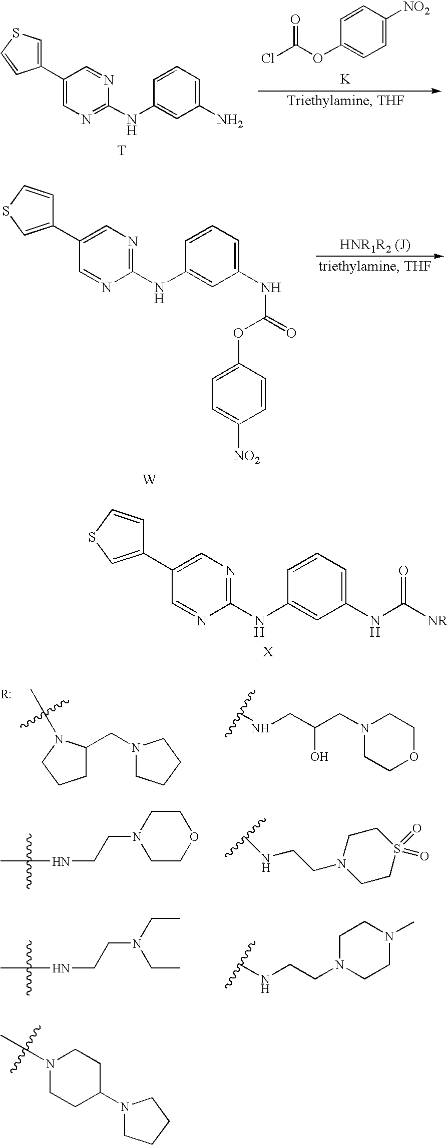

### [3-(5-Thiophen-3-yl-pyrimidin-2-ylamino)-phenyl]-carbamic acid 4-nitro-phenyl ester (W)

To a solution of N-(5-Thiophen-3-yl-pyrimidin-2-yl)-benzene-1,3-diamine (1 g, 3.7 mmol) in dry CH₂Cl₂ and pyridine (300 μL, 3.7 mmol) was added K (750 mg, 3.7 mmol) and then stirred at RT for 13 hours. The solution was cooled to 0° C., diluted with CH₂Cl₂ (100 mL) and washed with NaHCO₃ (1×100 mL), water (1×100 mL) and brine (1×100 mL). The organics were removed in vacuo yielding W in 43% yield as a yellow solid which required no further purification. ¹H NMR (300 MHz, DMSO-d₆) δ 7.11 (d, 1H), 7.28 (t, 1H), 7.43 (d, 1H), 7.55 (d, 2H), 7.58 (dt, 2H), 7.72-7.74 (m, 2H), 7.85 (d, 1H), 8.15 (d, 1H), 8.44 (d, 2H), 9.10 (s, 2H), 9.85 (s, 1H), 10.41 (s, 1H).

### Example 47

### N-(3-(5-(thiophen-3-yl)pyrimidin-2-ylamino)phenyl)-2-(2-((pyrrolidin-1-yl)methyl)pyrrolidin-1-yl)acetamide

To a stirring solution of U (200 mg, 0.58 mmol) in CH₃CN (100 mL) was added J (570 μL, 3.48 mmol) followed by diisopropylethylamine (300 μL, 1.74 mmol). The solution was brought to reflux and monitored by tlc analysis. After 13 hours the solvent was removed in vacuo and the residue purified via column chromatography (silica gel, 9:1 CH₂Cl₂/MeOH). The fractions containing product were evaporated to dryness under vacuum to yield compound 47 in 56% yield as a white crystalline solid. ¹H NMR (300 MHz, CDCl₃) δ 1.8-2.1 (m, 9H), 2.3-3.8 (m, 1H), 7.3-7.5 (m, 6H), 8.21 (d, 1H), 8.6 (s, 1H), 9.8 (brs, 1H). MS m/z 463 [M⁺+1].

### Example 48

### 2-pyrrolidin-1-ylmethyl-pyrrolidine-1-carboxylic acid [3-(5-thien-3-ylpyrimidin-2-yl)amino)-phenyl]-amide

To a stirring solution of W (100 mg, 0.23 mmol) in CH₂Cl₂ (20 mL) was added J (38 μL, 0.23 mmol) followed by triethylamine (32 mL, 0.23 mmol). The solution was stirred at room temperature for 12 hours where the reaction was diluted with CH₂Cl₂ (50 mL) and washed with 2N NaOH (2×50 mL), water (2×50 mL) and brine (2×50 mL). The organics were dried over Na₂SO₄ and concentrated in vacuo. The residue was purified using column chromatography (silica gel, 5% MeOH in CH₂Cl₂). The fractions containing product were evaporated to dryness under vacuum to yield compound 48 in 59% yield as a white solid. ¹H NMR (300 MHz, CDCl₃) δ 1.68-2.18 (m, 8H), 2.52-2.57 (m, 2H), 2.63-2.39 (m, 4H), 3.69-3.72 (m, 1H), 4.86-4.88 (m, 1H), 7.17-7.19 (m, 1H), 7.41-7.54 (m, 2H), 7.62 (d, 1H), 7.81 (d, 1H), 7.91 (d, 1H), 7.95 (d, 1H), 8.69 (s, 2H) MS m/z 449 [M⁺+1].

### Example 49

### 1-(2-morpholin-4-yl-ethyl)-3-[3-(5-thien-3-ylpyrimidin-2-yl)amino)-phenyl]-urea

To a stirring solution of W (100 mg, 0.23 mmol) in CH₂Cl₂ (20 mL) was added J (29 mg, 0.23 mmol) followed by triethylamine (32 μL, 0.23 mmol). The solution was stirred at room temperature for 12 hours where the reaction was diluted with CH₂Cl₂ (50 mL) and washed with 2N NaOH (2×50 mL), water (2×50 mL) and brine (2×50 mL). The organics were dried over Na₂SO₄ and concentrated in vacuo. The residue was purified using column chromatography (silica gel, 5% MeOH in CH₂Cl₂). The fractions containing product were evaporated to dryness under vacuum to yield compound 49 in 44% yield as a white solid. ¹H NMR (300 MHz, CD₃OD) δ 2.52-3.36 (m, 6H), 3.29-3.36 (m, 6H), 7.01-7.04 (m, 1H), 7.15-7.28 (m, 3H), 7.42-7.65 (m, 2H), 7.85-7.86 (m, 2H), 8.71-8.72 (s, 2H). MS m/z 425 [M⁺+1].

### Example 50

### 1-(2-diethylamino-ethyl)-3-[3-(5-thien-3-ylpyrimidin-2-yl)amino)-phenyl]-urea

To a stirring solution of W (100 mg, 0.23 mmol) in CH₂Cl₂ (20 mL) was added J (27 mg, 0.23 mmol) followed by triethylamine (32 mL, 0.23 mmol). The solution was stirred at room temperature for 12 hours where the reaction was diluted with CH₂Cl₂ (50 mL) and washed with 2N NaOH (2×50 mL), water (2×50 mL) and brine (2×50 mL). The organics were dried over Na₂SO₄ and concentrated in vacuo. The residue was purified using column chromatography (silica gel, 5% MeOH in CH₂Cl₂). The fractions containing product were evaporated to dryness under vacuum to yield compound 50 in 59% yield as a white solid. ¹H NMR (300 MHz, CD₃OD) δ 1.08 (t, J, 7.1 Hz, 6H), 2.64 (q, J, 7.1 Hz, 2H), 3.29-3.34 (m, 4H), 7.04-7.05 (d, 1H), 7.25-7.26 (t, 1H), 7.41-7.42 (d, 1H), 7.43-7.44 (d, 1H), 7.51-7.52 (d, 1H), 7.62-7.63 (d, 1H), 7.85-7.85 (d, 1H), 8.71 (s, 2H). MS m/z 411 [M⁺+1].

### Example 51

### 1-(2-hydroxy-3-morpholin-4-yl-propyl)-3-[3-(5-thiophen-3-yl-pyrimidin-2-ylamino)-phenyl]-urea

To a stirring solution of W (100 mg, 0.23 mmol) in CH₂Cl₂ (20 mL) was added J (41 mg, 0.23 mmol) followed by triethylamine (32 μL, 0.23 mmol). The solution was stirred at room temperature for 12 hours where the reaction was diluted with CH₂Cl₂ (50 mL) and washed with 2N NaOH (2×50 mL), water (2×50 mL) and brine (2×50 mL). The organics were dried over Na₂SO₄ and concentrated in vacuo. The residue was purified using column chromatography (silica gel, 5% MeOH in CH₂Cl₂). The fractions containing product were evaporated to dryness under vacuum to yield compound 51 in 24% yield as a white solid. ¹H NMR (300 MHz, CD₃OD) δ 2.41 (d, 1H), 2.51-2.53 (m, 6H), 3.67-3.70 (m, 6H), 3.92-4.11 (m, 1H), 0.04-7.05 (d, 1H), 7.25-7.26 (t, 1H), 7.41-7.42 (d, 1H), 7.43-7.44 (d, 1H), 7.51-7.52 (d, 1H), 7.621-7.63 (d, 1H), 7.85-7.86 (d, 1H), 8.71 (s, 2H). MS m/z 455 [M⁺+1].

### Example 52

### 1-[2-(1,1-dioxo-1λ⁶-thiomorpholin-4-yl)ethyl]-3-[3-(5-thiophen-3-yl-pyrimidin-2-ylamino)-phenyl]-urea

To a stirring solution of W (100 mg, 0.23 mmol) in CH₂Cl₂ (20 mL) was added J (41 mg, 0.23 mmol) followed by triethylamine (32 μL, 0.23 mmol). The solution was stirred at room temperature for 12 hours where the reaction was diluted with CH₂CO₂ (50 mL) and washed with 2N NaOH (2×50 mL), water (2×50 mL) and brine (2×50 mL). The organics were dried over Na₂SO₄ and concentrated in vacuo. The residue was purified using column chromatography (silica gel, 5% MeOH in CH₂Cl₂). The fractions containing product were evaporated to dryness under vacuum to yield compound 52 in 50% yield as a white solid. ¹H NMR (300 MHz, CD₃OD) δ 2.68 (t, J=6.1 Hz, 2H), 3.06-3.35 (m, 4H), 4.79-4.87 (m, 6H), 7.01-7.02 (m, 1H), 7.04-7.05 (m, 2H), 7.43 (d, 1H), 7.45 (d, 1H), 7.64 (d, 1H), 7.88 (d, 1H), 8.72 (s, 2H). MS m/z 473 [M⁺+1].

### Example 53

### 1-[2-(4-methyl-piperazin-1-yl)-ethyl]-3-[3-(5-thiophen-3-yl-pyrimidin-2-ylamino)-phenyl]-urea

To a stirring solution of W (100 mg, 0.23 mmol) in CH₂Cl₂ (20 mL) was added J (33 mg, 0.23 mmol) followed by triethylamine (32 μL, 0.23 mmol). The solution was stirred at room temperature for 12 hours where the reaction was diluted with CH₂Cl₂ (50 mL) and washed with 2N NaOH (2×50 mL), water (2×50 mL) and brine (2×50 mL). The organics were dried over Na₂SO₄ and concentrated in vacuo. The residue was purified using column chromatography (silica gel, 5% MeOH in CH₂Cl₂). The fractions containing product were evaporated to dryness under vacuum to yield compound 53 in 45% yield as a white solid. ¹H NMR (300 MHz, CD₃OD) δ 2.27 (s, 3H), 2.33-2.55 (m, 6H), 7.01-7.02 (m, 1H), 7.04-7.05 (m, 2H), 7.43 (d, 1H), 7.45 (d, 1H), 7.64 (d, 1H), 7.86 (d, 1H), 8.72 (s, 2H). MS m/z 438 [M⁺+1].

### Example 54

### 4-pyrrolidin-1-yl-piperidine-1-carboxylic acid [3-(5-thiophen-3-yl-pyrimidin-2-ylamino)-phenyl]-amide

To a stirring solution of W (100 mg, 0.23 mmol) in CH₂Cl₂ (20 mL) was added J (35 mg, 0.23 mmol) followed by triethylamine (32 μL, 0.23 mmol). The solution was stirred at room temperature for 12 hours where the reaction was diluted with CH₂Cl₂ (50 mL) and washed with 2N NaOH (2×50 mL), water (2×50 mL) and brine (2×50 mL). The organics were dried over Na₂SO₄ and concentrated in vacuo. The residue was purified using column chromatography (silica gel, 5% MeOH in CH₂Cl₂). The fractions containing product were evaporated to dryness under vacuum to yield compound 54 in 72% yield as a white solid. ¹H NMR (300 MHz, CD₃OD) δ 1.06 (t, J, 1.5 Hz, 1H), 1.43-1.48 (m, 2H), 1.80 (brs, 3H), 2.28-2.29 (m, 1H), 2.62-2.64 (m, 3H), 3.30 (t, 2H), 4.18 (d, J, 14 Hz, 2H), 7.00 (d, J, 7.7 Hz, 1H), 7.18 (t, J, 8 Hz, 1H), 7.31 (d, J, 7.8 Hz, 1H), 7.41 (d, J, 1.3 Hz, 1H), 7.43 (d, J, 1.3 Hz, 1H), 7.62 (d, J, 1.4 Hz, 1H), 7.83 (d, J, 1.9 Hz, 1H), 8.70 (d, J, 1.5 Hz, 2H). MS m/z 449 [M⁺+1].

### Example 55

### 2-(4-pyrrolidin-1-yl-Piperidine-1-yl)-N-[3-(5-thiophen-3-yl-pyrimidin-2-ylamino)-phenyl]-acetamide

To a stirring solution of U (200 mg, 0.58 mmol) in CH₃CN (100 mL) was added J (535 mg, 3.48 mmol) followed by diisopropylethylamine (300 μL, 1.74 mmol). The solution was brought to reflux and monitored by tlc analysis. After 13 hours the solvent was removed in vacuo and the residue purified via column chromatography (silica gel, 9:1 CH₂Cl₂/MeOH). The fractions containing product were evaporated to dryness under vacuum to yield compound 55 in 52% yield as a white crystalline solid. ¹H NMR (300 MHz, CDCl₃) δ 1.60-2.02 (brm, 10H), 2.21-2.41 (brt, 2H), 2.4-2.8 (brs, 4H), 3.0-3.10 (brd, 2H), 3.12 (s, 2H), 7.20-7.45 (m, 10H), 8.08 (s, 1H), 8.70 (s, 2H), 8.5 (s, 1H). MS m/z 463 [M⁺+1].

### Example 56

### 2-(2-morpholin-4-yl-ethylamino)-N-[3-(5-thiophen-3-yl-pyrimidin-2-ylamino)-phenyl]-acetamide

To a stirring solution of U (200 mg, 0.58 mmol) in CH₃CN (100 mL) was added J (452 mg, 3.48 mmol) followed by diisopropylethylamine (300 μL, 1.74 mmol). The solution was brought to reflux and monitored by tlc analysis. After 13 hours the solvent was removed in vacuo and the residue purified via column chromatography (silica gel, 9:1 CH₂Cl₂/MeOH). The fractions containing product were evaporated to dryness under vacuum to yield compound 56 in 88% yield as a white crystalline solid. ¹H NMR (300 MHz, CDCl₃) δ 2.35-2.45 (m, 6H), 2.51-2.53 (m, 2H), 3.40 (s, 2H), 3.70-3.73 (m, 6H), 7.2-7.4 (m, 8H), 8.1 (s, 1H), 8.67 (s, 2H). MS m/z 439 [M⁺+1].

### Example 57

### 2-(2-diethylamino-ethylamino)-N-[3-(5-thiophen-3-yl-pyrimidin-2-ylamino)-phenyl]-acetamide

To a stirring solution of U (200 mg, 0.58 mmol) in CH₃CN (100 mL) was added J (403 μL, 3.48 mmol) followed by diisopropylethylamine (300 μL, 1.74 mmol). The solution was brought to reflux and monitored by tlc analysis. After 13 hours the solvent was removed in vacuo and the residue purified via column chromatography (silica gel, 9:1 CH₂Cl₂/MeOH). The fractions containing product were evaporated to dryness under vacuum to yield compound 57 in 72% yield as a white crystalline solid. ¹H NMR (300 MHz, CDCl₃) δ 1.21 (t, J, 7.1 Hz, 6H), 2.8-3.4 (m, 8H), 3.46 (s, 2H), 7.2-7.4 (m, 6H), 8.12 (d, 1H), 9.60 (s, 2H), 9.80 (s, 1H). MS m/z 425 [M⁺+1].

### Example 58

### 2-[2-(4-methyl-piperazin-1-yl)-ethylamino]-N-[3-(5-thiophen-3-yl-pyrimidin-2-ylamino)-phenyl]-acetamide

To a stirring solution of U (200 mg, 0.58 mmol) in CH₃CN (100 mL) was added J (486 mg, 3.48 mmol) followed by diisopropylethylamine (300 μL, 1.74 mmol). The solution was brought to reflux and monitored by tlc analysis. After 13 hours the solvent was removed in vacuo and the residue purified via column chromatography (silica gel, 9:1 CH₂Cl₂/MeOH). The fractions containing product were evaporated to dryness under vacuum to yield compound 58 in 44% yield as a white crystalline solid. ¹H NMR (300 MHz, CDCl₃) δ 2.27 (s, 3H), 2.27-2.46 (m, 10H), 2.5-2.54 (m, 2H), 3.39 (s, 2H), 7.2-7.46 (m, 1H), 8.09 (s, 1H), 8.67 (s, 2H), 9.42 (s, 1H). MS m/z 452 [M⁺+1].

### Example 59

### 2-(2-hydroxy-3-morpholin-4-yl-propylamino)-N-[3-(5-thiophen-3-yl-pyrimidin-2-ylamino)-phenyl]-acetamide

To a stirring solution of U (200 mg, 0.58 mmol) in CH₃CN (100 mL) was added J (544 mg, 3.48 mmol) followed by diisopropylethylamine (300 μL, 1.74 mmol). The solution was brought to reflux and monitored by tlc analysis. After 13 hours the solvent was removed in vacuo and the residue purified via column chromatography (silica gel, 9:1 CH₂Cl₂/MeOH). The fractions containing product were evaporated to dryness under vacuum to yield compound 59 in 50% yield as a white crystalline solid. ¹H NMR (300 MHz, CDCl₃) δ 2.37-2.67 (m, 12H), 3.42-3.46 (m, 3H), 3.69-3.72 (m, 6H), 7.25-7.43 (m, 8H), 8.09 (s, 1H), 8.67 (s, 1H). MS m/z 469 [M⁺+1].

### Example 60

### 2-[2-(1,1-dioxo-1λ⁶-thiomorpholin-4-yl)-ethylamino]-N-[3-(5-thiophen-3-yl-pyrimidin-2-ylamino)-Phenyl]-acetamide

To a stirring solution of U (200 mg, 0.58 mmol) in CH₃CN (100 mL) was added J (619 mg, 3.48 mmol) followed by diisopropylethylamine (300 μL, 1.74 mmol). The solution was brought to reflux and monitored by tlc analysis. After 13 hours the solvent was removed in vacuo and the residue purified via column chromatography (silica gel, 9:1 CH₂Cl₂/MeOH). The fractions containing product were evaporated to dryness under vacuum to yield compound 60 in 39% yield as a white crystalline solid. ¹H NMR (300 MHz, CDCl₃) δ 2.67-2.79 (m, 4H), 3.05 (brs, 8H), 3.42 (s, 2H), 7.16-7.70 (m, 8H), 8.10 (s, 1H), 8.68 (s, 2H), 9.22 (s, 1H). MS m/z 487 [M⁺+1].

### Example 61

### 2-morpholin-4-yl-N-[3-(5-thiophen-3-yl-pyrimidin-2-ylamino)-phenyl]-acetamide

To a stirring solution of U (200 mg, 0.58 mmol) in CH₃CN (100 mL) was added J (150 μL, 3.48 mmol) followed by diisopropylethylamine (300 μL, 1.74 mmol). The solution was brought to reflux and monitored by tlc analysis. After 13 hours the solvent was removed in vacuo and the residue purified via column chromatography (silica gel, 9:1 CH₂Cl₂/MeOH). The fractions containing product were evaporated to dryness under vacuum to yield compound 61 in 60% yield as a white crystalline solid. ¹H NMR (300 MHz, CDCl₃) δ 2.63 (t, J, 4.5 Hz, 4H), 3.15 (s, 2H), 3.79 (t, J, 4.5 Hz, 4H), 7.1-7.6 (m, 8H), 8.01 (s, 1H), 8.11 (s, 2H), 8.69 (s, 1H). MS m/z 396 [M⁺+1].

### Example 62

### 2-Pyrrolidin-1-yl-N-[3-(5-thiophen-3-yl-pyrimidin-2-ylamino)-phenyl]-acetamide

To a stirring solution of U (200 mg, 0.58 mmol) in CH₃CN (100 mL) was added J (140 μL, 3.48 mmol) followed by diisopropylethylamine (300 μL, 1.74 mmol). The solution was brought to reflux and monitored by tlc analysis. After 13 hours the solvent was removed in vacuo and the residue purified via column chromatography (silica gel, 9:1 CH₂Cl₂/MeOH). The fractions containing product were evaporated to dryness under vacuum to yield compound 62 in 89% yield as a white crystalline solid. ¹H NMR (300 MHz, CDCl₃) δ 1.83 (t, J, 4.5 Hz, 4H), 2.67 (t, J, 4.5 Hz, 4H), 3.2 (s, 2H), 7.1-7.6 (m, 8H), 8.01 (s, 1H), 8.11 (s, 2H), 8.69 (s, 1H). MS m/z 380 [M⁺+1].

### Biological Examples

### A. Assay Procedures

The following assays may be used to determine the level of activity and effect of the different compounds of the present invention on one or more of the PKs. Similar assays can be designed along the same lines for any PK using techniques well known in the art.

The assays described herein are performed in an ELISA (Enzyme-Linked Immunosorbent Sandwich Assay) format (Voller, et al., 1980, “Enzyme-Linked Immunosorbent Assay,” Manual of Clinical Immunology, 2d ed., Rose and Friedman, Am. Soc. Of Microbiology, Washington, D.C., pp. 359-371). The general procedure is as follows: a compound is introduced to cells expressing the test kinase, either naturally or recombinantly, for a selected period of time after which, if the test kinase is a receptor, a ligand known to activate the receptor is added. The cells are lysed and the lysate is transferred to the wells of an ELISA plate previously coated with a specific antibody recognizing the substrate of the enzymatic phosphorylation reaction. Non-substrate components of the cell lysate are washed away and the amount of phosphorylation on the substrate is detected with an antibody specifically recognizing phosphotyrosine compared with control cells that were not contacted with a test compound.

The presently preferred protocols for conducting the ELISA experiments for specific PKs is provided below. However, adaptation of these protocols for determining the activity of compounds against other RTKs, as well as for CTKs and STKs, is well within the scope of knowledge of those skilled in the art. Other assays described herein measure the amount of DNA made in response to activation of a test kinase, which is a general measure of a proliferative response. The general procedure for this assay is as follows: a compound is introduced to cells expressing the test kinase, either naturally or recombinantly, for a selected period of time after which, if the test kinase is a receptor, a ligand known to activate the receptor is added. After incubation at least overnight, a DNA labeling reagent such as 5-bromodeoxyuridine (BrdU) or H³-thymidine is added. The amount of labeled DNA is detected with either an anti-BrdU antibody or by measuring radioactivity and is compared to control cells not contacted with a test compound.

GST-FLK-1 Bioassay

This assay analyzes the tyrosine kinase activity of GST-Flk1 on poly(glu,tyr) peptides.

Materials and Reagents:

1. Corning 96-well ELISA plates (Corning Catalog No. 5805-96).
2. poly(glu,tyr) 4:1, lyophilizate (Sigma Catalog #P0275).
3. Preparation of poly(glu,tyr)(pEY) coated assay plates: Coat 2 ug/well of poly(glu,tyr)(pEY) in 100 μL PBS, hold at room temperature for 2 hours or at 4° C. overnight. Cover plates well to prevent evaporation.
4. PBS Buffer: for 1 L, mix 0.2 g KH₂PO₄, 1.15 g Na₂HPO₄, 0.2 g KCl and 8 g NaCl in approx. 900 ml dH₂O. When all reagents have dissolved, adjust the pH to 7.2 with HCl. Bring total volume to 1 L with dH₂O.
5. PBST Buffer: to 1 L of PBS Buffer, add 1.0 ml Tween-20.
6. TBB—Blocking Buffer: for 1 L, mix 1.21 g TRIS, 8.77 g NaCl, 1 ml TWEEN-20 in approximately 900 ml dH₂O. Adjust pH to 7.2 with HCl. Add 10 g BSA, stir to dissolve. Bring total volume to 1 L with dH₂O. Filter to remove particulate matter.
7. 1% BSA in PBS: To make a 1× working solution, add 10 g BSA to approx. 990 ml PBS buffer, stir to dissolve. Adjust total volume to 1 L with PBS buffer, filter to remove particulate matter.
8. 50 mM Hepes pH 7.5.
9. GST-Flk1cd purified from sf9 recombinant baculovirus transformation (SUGEN, Inc.).
10. 4% DMSO in dH₂O.
11. 10 mMATP in dH₂O.
12. 40 mM MnCl₂
13. Kinase Dilution Buffer (KDB): mix 10 ml Hepes (pH 7.5), 1 ml 5M NaCl, 40 μL 100 mM sodium orthovanadate and 0.4 ml of 5% BSA in dH₂O with 88.56 ml dH₂O.
14. NUNC 96-well V bottom polypropylene plates, Applied Scientific Catalog #AS-72092
15. EDTA: mix 14.12 g ethylenediaminetetraacetic acid (EDTA) to approx. 70 ml dH₂O. Add 10 N NaOH until EDTA dissolves. Adjust pH to 8.0. Adjust total volume to 100 ml with dH₂O.
16. 1⁰ Antibody Dilution Buffer: mix 10 ml of 5% BSA in PBS buffer with 89.5 ml TBST.
17. Anti-phosphotyrosine monoclonal antibody conjugated to horseradish peroxidase (PY99 HRP, Santa Cruz Biotech).
18. 2,2′-Azinobis(3-ethylbenzthiazoline-6-sulfonic acid (ABTS, Moss, Cat. No. ABST).
19. 10% SDS.
   Procedure:
1. Coat Corning 96-well ELISA plates with 2 μg of polyEY peptide in sterile PBS as described in step 3 of Materials and Reagents.
2. Remove unbound liquid from wells by inverting plate. Wash once with TBST. Pat the plate on a paper towel to remove excess liquid.
3. Add 100 μl of 1% BSA in PBS to each well. Incubate, with shaking, for 1 hr. at room temperature.
4. Repeat step 2.
5. Soak wells with 50 mM HEPES (pH7.5) (150 μl/well).
6. Dilute test compound with dH₂O/4% DMSO to 4 times the desired final assay concentration in 96-well polypropylene plates.
7. Add 25 μl diluted test compound to ELISA plate. In control wells, place 25 μl of dH₂O/4% DMSO.
8. Add 25 μl of 40 mM MnCl₂ with 4×ATP (2 μM) to each well.
9. Add 25 μl 0.5M EDTA to negative control wells.
10. Dilute GST-Flk1 to 0.005 μg (5 ng)/well with KDB.
11. Add 50 μl of diluted enzyme to each well.
12. Incubate, with shaking, for 15 minutes at room temperature.
13. Stop reaction by adding 50 μl of 250 mM EDTA (pH 8.0).
14. Wash 3× with TBST and pat plate on paper towel to remove excess liquid.
15. Add 100 μl per well anti-phosphotyrosine HRP conjugate, 1:5,000 dilution in antibody dilution buffer. Incubate, with shaking, for 90 min. at room temperature.
16. Wash as in step 14.
17. Add 100 μl of room temperature ABTS solution to each well.
18. Incubate, with shaking, for 10 to 15 minutes. Remove any bubbles.
19. Stop reaction by adding 20 μl of 10% SDS to each well.
20. Read results on Dynatech MR7000ELISA reader with test filter at 410 nM and reference filter at 630 nM.
   PDGFR Bioassay

This assay is used to the in vitro kinase activity of PDGFR in an ELISA assay.

Materials and Reagents:

1. Corning 96-well Elisa plates
2. 28D4C10 monoclonal anti-PDGFR antibody (SUGEN, Inc.).
3. PBS.
4. TBST Buffer.
5. Blocking Buffer (same as for EGFR bioassay).
6. PDGFR-β expressing NIH 3T3 cell lysate (SUGEN, Inc.).
7. TBS Buffer.
8. TBS+10% DMSO.
9. ATP.
10. MnCl₂.
11. Kinase buffer phosphorylation mix: for 10 ml, mix 250 μl 1 M TRIS, 200 μl 5M NaCl, 100 μl 1 M MnCl₂ and 50 g 100 mM Triton X-100 in enough dH₂O to make 10 ml.
12. NUNC 96-well V bottom polypropylene plates.
13. EDTA.
14. Rabbit polyclonal anti-phosphotyrosine serum (SUGEN, Inc.).
15. Goat anti-rabbit IgG peroxidase conjugate (Biosource Cat. No. AL10404).
16. ABTS.
17. Hydrogen peroxide, 30% solution.
18. ABTS/H₂O₂.
19. 0.2 M HCl.
   Procedure:
1. Coat Corning 96 well ELISA plates with 0.5 μg 28D4C10 in 100 μl PBS per well, store overnight at 4° C.
2. Remove unbound 28D4C10 from wells by inverting plate to remove liquid. Wash 1× with dH₂O. Pat the plate on a paper towel to remove excess liquid.
3. Add 150 μl of Blocking Buffer to each well. Incubate for 30 min. at room temperature with shaking.
4. Wash plate 3× with deionized water, then once with TBST. Pat plate on a paper towel to remove excess liquid and bubbles.
5. Dilute lysate in HNTG (10 μg lysate/100b HNTG).
6. Add 100 μl of diluted lysate to each well. Shake at room temperature for 60 min.
7. Wash plates as described in Step 4.
8. Add 80 μl working kinase buffer mix to ELISA plate containing captured PDGFR.
9. Dilute test compound 1:10 in TBS in 96-well polypropylene plates.
10. Add 10 μl diluted test compound to ELISA plate. To control wells, add 10 μl TBS+10% DMSO. Incubate with shaking for 30 minutes at room temperature.
11. Add 10 μl ATP directly to all wells except negative control well (final well volume should be approximately 100 μl with 20 μM ATP in each well.) Incubate 30 minutes with shaking.
12. Stop reaction by adding 10 μl of EDTA solution to each well.
13. Wash 4× with deionized water, twice with TBST.
14. Add 100 μl anti-phosphotyrosine (1:3000 dilution in TBST) per well. Incubate with shaking for 30-45 min. at room temperature.
15. Wash as in Step 4.
16. Add 100 μl Biosource Goat anti-rabbit IgG peroxidase conjugate (1:2000 dilution in TBST) to each well. Incubate with shaking for 30 min. at room temperature.
17. Wash as in Step 4.
18. Add 100 μl of ABTS/H₂O₂ solution to each well.
19. Incubate 10 to 30 minutes with shaking. Remove any bubbles.
20. If necessary stop reaction with the addition of 100 μl 0.2 M HCl per well.
21. Read assay on Dynatech MR7000ELISA reader with test filter at 410 nM and reference filter at 630 nM.

All patents and publications are herein incorporated by reference to the same extent as if each individual publication was specifically and individually indicated to be incorporated by reference.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described.

1. A compound of the structure: 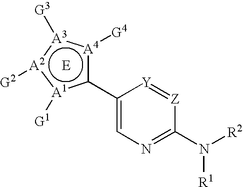
wherein:
one of Y and Z is N and the other is C;
in the ring E, one of A¹, A², A³ and A⁴ is S and the others are C; and
each G¹, G², G³ and G⁴ is independently H or an R⁵ group, except that the one of A1, A2, A³ and A⁴ that is S has no G group attached, and two adjacent G groups can optionally combine to form a 5 or 6 membered aryl, heteroaryl, aliphatic or heteroaliphatic ring;
R¹ is H or C₁₋₆ alkyl;
R² is (i) a six membered aryl or five or six membered heteroaryl, optionally fused with another five or six membered aryl or heteroaryl to form a naphthalene, indene, pentalene or a fused bicyclic heteroaryl, or (ii) —C(═O)NH₂; and wherein each hydrogen in R² independently is optionally substituted by C₁₋₆ alkyl, C₁₋₆ alkenyl, C₁₋₆ akynyl, C₃₋₁₂ cycloalkyl, C₆₋₁₂ aryl, 6 to 12-membered heteroaryl, 3 to 12-membered heteroalicyclic, halogen, hydroxy, C₁₋₆ alkoxy, trihalomethanecarbonyl, sulfonyl, trihalomethanesulfonyl, C-carboxyl, O-carboxyl, C-amido, —OR³—COR³, —CONR³R⁴, —COOR³, —NR³R⁴, —CN, —NO₂, —S(O)ₙR³, —S(O₂)NR³R⁴, —NR³R⁴, perfluoro-C₁₋₆ alkyl, —NR³ONR³R⁴, —NR³OR⁴ or —NR³S(O)₂R⁴;
each R³ and R⁴ is independently hydrogen, C₁₋₆ alkyl, C₃₋₁₂ cycloalkyl, C₆₋₁₂ aryl, carbonyl, acetyl, sulfonyl, or trifluoromethanesulfonyl, and R³ and R⁴ are optionally combined to form a 5 or 6-membered heteroalicyclic group;
each R⁵ is independently C₁₋₆ alkyl, C₁₋₆ alkenyl, C₁₋₆ akynyl, C₃₋₁₂ cycloalkyl, C₆₋₁₂ aryl, 6 to 12-membered heteroaryl, 3 to 12-membered heteroalicyclic, halogen, hydroxy, C₁₋₆ alkoxy, trihalomethanecarbonyl, sulfonyl, trihalomethanesulfonyl, C-carboxyl, O-carboxyl, C-amido, —OR³, —COR³, —CONR³R⁴, —COOR³, —NR³R⁴, —CN, —NO₂, —S(O)ₙR³, —S(O₂)NR³R⁴, —NR³R⁴, perfluoro-C₁₋₆ alkyl, —NR³ONR³R⁴, —NR³OR⁴ or —NR³S(O)₂R⁴; and
n is 0, 1 or 2,
or a pharmaceutically acceptable salt, solvate or hydrate thereof.

2. The compound of claim 1, wherein R¹═ is H.

3. The compound of claim 1, wherein R² is C(═O)NH₂, and one of the hydrogen atoms in R² is optionally substituted by C₁₋₆ alkyl, C₁₋₆ alkenyl, C₁₋₆ akynyl, C₃₋₁₂ cycloalkyl, C₆₋₁₂ aryl, 6 to 12-membered heteroaryl, 3 to 12-membered heteroalicyclic, halogen, hydroxy, C₁₋₆ alkoxy, trihalomethanecarbonyl, sulfonyl, trihalomethanesulfonyl, C-carboxyl, O-carboxyl, C-amido, —OR³, —COR³, —CONR³R⁴, —COOR³, —NR³R⁴, —CN, —NO₂, —S(O)ₙR³, —S(O₂)NR³R⁴, —NR³R⁴, perfluoro-C₁₋₆ alkyl, —NR³ONR³R⁴, —NR³OR⁴ or —NR³S(O)₂R⁴.

4. The compound of claim 1, wherein G¹, G², G³ and G⁴ are H.

5. The compound of claim 1, wherein R² is a substituted or unsubstituted phenyl.

6. The compound of claim 1, wherein R² is a phenyl substituted at the 3 or 4 position.

7. The compound of claim 1, wherein Z is N and Y is C.

8. The compound of claim 1, wherein Z is C and Y is N.

9. The compound of claim 1, wherein R² is chosen from thiophene, pyrrole, pyrazole, imidazole, 1,2,3-triazole, 1,2,4-triazole, oxazole, isoxazole, thiazole, isothiazole, 2-sulfonylfuran, 1,2,3-oxadiazole, 1,2,4-oxadiazole, 1,2,5-oxadiazole, 1,3,4-oxadiazole, 1,2,3,4-oxatriazole, 1,2,3,5-oxatriazole, 1,2,3-thiadiazole, 1,2,4-thiadiazole, 1,2,5-thiadiazole, 1,3,4-thiadiazole, 1,2,3,4-thiatriazole, 1,2,3,5-thiatriazole, tetrazole, pyridine, pyrazine, pyrimidine, pyridazine, pyran, benzothiophene, isobenzothiophene, benzofuran, isobenzofuran, chromene, isochromene, indolizine, isoindole, 3H-indole, indole, indazole, purine, 4H-quinolizine, isoquinole, quinole, phthalazine, naphthyridine, quinoxaline, quinazoline, cinnoline, and pteridine.

10. A compound of the structure: 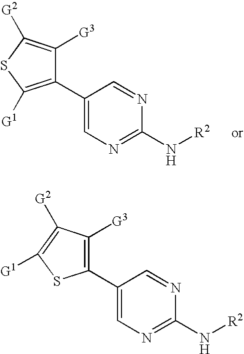
wherein:
each G¹, G² and G³ is independently H or an R⁵ group, and two adjacent G groups can optionally combine to form a 5 or 6 membered aryl, heteroaryl, aliphatic or heteroaliphatic ring;
R² is phenyl or —C(═O)NH₂; and each hydrogen in R² independently is optionally substituted by C₁₋₆ alkyl, C₁₋₆ alkenyl, C₁₋₆ akynyl, C₃₋₁₂ cycloalkyl, C₆₋₁₂ aryl, 6 to 12-membered heteroaryl, 3 to 12-membered heteroalicyclic, halogen, hydroxy, C₁₋₆ alkoxy, trihalomethanecarbonyl, sulfonyl, trihalomethanesulfonyl, C-carboxyl, O-carboxyl, C-amido, —OR³, —COR³, —CONR³R⁴, —COOR³, —NR³R⁴, —CN, —NO₂, —S(O)ₙR³, —S(O₂)NR³R⁴, —NR³R⁴, perfluoro-C₆₋₄ alkyl, —NR³ONR³R⁴, —NR³OR⁴ or —NR³S(O)₂R⁴;
each R³ and R⁴ is independently hydrogen, C₁₋₆ alkyl, C₃₋₁₂ cycloalkyl, C₆₋₁₂ aryl, carbonyl, acetyl, sulfonyl, or trifluoromethanesulfonyl, and R³ and R⁴ are optionally combined to form a 5 or 6-membered heteroalicyclic group;
each R⁵ is independently C₁₋₆ alkyl, C₁₋₆ alkenyl, C₁₄ akynyl, C₃₋₁₂ cycloalkyl, C₆₋₁₂ aryl, 6 to 12-membered heteroaryl, 3 to 12-membered heteroalicyclic, halogen, hydroxy, C₁ alkoxy, trihalomethanecarbonyl, sulfonyl, trihalomethanesulfonyl, C-carboxyl, O-carboxyl, C-amido, —OR³—COR³, —CONR³R⁴, —COOR³, —NR³R⁴, —CN, —NO₂, —S(O)ₙR³, —S(O₂)NR³R⁴, —NR³R⁴, perfluoro-C₁₋₆ alkyl, —NR³ONR³R⁴—NR³OR⁴ or —NR³S(O)₂R⁴; and
n is 0, 1 or 2,
or a pharmaceutically acceptable salt, solvate or hydrate thereof.

11. The compound of claim 10, wherein R² is C(═O)NH₂, and one of the hydrogen atoms in R² is optionally substituted by C₁₋₆ alkyl, C₁₋₆ alkenyl, C₁₋₆ akynyl, C₃₋₁₂ cycloalkyl, C₆₋₁₂ aryl, 6 to 12-membered heteroaryl, 3 to 12-membered heteroalicyclic, halogen, hydroxy, C₁₋₆ alkoxy, trihalomethanecarbonyl, sulfonyl, trihalomethanesulfonyl, C-carboxyl, O-carboxyl, C-amido, —OR³, —COR³, —CONR³R⁴, —COOR³, —NR³R⁴, —CN, —NO₂, —S(O)ₙR³—S(O₂)NR³R⁴, —NR³R⁴, perfluoro-C₁₋₆ alkyl, —NR³ONR³R⁴, —NR³OR⁴ or —NR³S(O)₂R⁴.

12. The compound of claim 10, wherein R² is a substituted or unsubstituted phenyl.

13. A compound selected from the group consisting of: 4-[(5-thien-3-ylpyrimidin-2-yl)amino]phenol; N-{4-[(5-thien-3-ylpyrimidin-2-yl)amino]phenyl}acetamide; N-(4-morpholin-4-ylphenyl)-5-thien-3-ylpyrimidin-2-amine; 4-amino-N-{4-[(5-thien-3-ylpyrimidin-2-yl)amino]phenyl}benzamide; N-(4-methoxyphenyl)-5-thien-3-ylpyrimidin-2-amine; N-(5-thien-3-ylpyrimidin-2-yl)benzene-1,3-diamine; 3-[(5-thien-3-ylpyrimidin-2-yl)amino]benzoic acid; N-(5-thien-3-ylpyrimidin-2-yl)benzene-1,4-diamine; N-(4-methoxyphenyl)-N′-(5-thien-3-ylpyrimidin-2-yl)urea; N-(4-fluorophenyl)-N′-(5-thien-3-ylpyrimidin-2-yl)urea; 4-[(4-methylpiperazin-1-yl)methyl]-N-{3-[(5-thien-3-ylpyrimidin-2-yl)amino]phenyl}benzamide; 4-[(4-methylpiperazin-1-yl)methyl]-N-{4-[(5-thien-3-ylpyrimidin-2-yl)amino]phenyl}benzamide; N-[4-(5-thien-2-ylpyrimidin-2-ylamino]phenyl}acetamide; N-{3-[(5-thien-2-ylpyrimidin-2-yl)amino]phenyl}acetamide; 4-(5-thiophen-2-yl-pyrimidin-2-ylamino)-phenol; 4-amino-N-{4-[(5-thien-2-ylpyrimidin-2-yl)amino]phenyl}benzamide; 4-[(5-thien-2-ylpyrimidin-2-yl)amino]benzoic acid; N-phenyl-3-[(5-thien-2-ylpyrimidin-2-yl)amino]benzamide; 1-(5-{2-[(3-aminophenyl)amino]pyrimidin-5-yl}thien-2-yl)ethanone; N-[5-(1-benzothien-3-yl)pyrimidin-2-yl]benzene-1,3-diamine; N-(3-(5-(thiophen-3-yl)pyrimidin-2-ylamino)phenyl)-2-(3-((pyrrolidin-1-yl)methyl)pyrrolidin-1-yl)acetamide; 2-[2-(pyrrolidin-1-ylmethyl)pyrrolidin-1-yl]-N-{4-[(5-thien-3-ylpyrimidin-2-yl)amino]phenyl}acetamide; 2-(4-methylpiperazin-1-yl)-N-{4-[(5-thien-3-ylpyrimidin-2-yl)amino]phenyl}acetamide; 2-(4-pyrrolidin-1-ylpiperidin-1-yl)-N-{4-[(5-thien-3-ylpyrimidin-2-yl)amino]phenyl}acetamide; 2-(2-morpholinoethylamino)-N-(4-(5-(thiophen-3-yl)pyrimidin-2-ylamino)phenyl)acetamide; 2-morpholin-4-yl-N-{4-[(5-thien-3-ylpyrimidin-2-yl)amino]phenyl}acetamide; N-(4-(5-(thiophen-3-yl)pyrimidin-2-ylamino)phenyl)-2-(diethylamino)acetamide; 2-(4-hydroxypiperidin-1-yl)-N-{4-[(5-thien-3-ylpyrimidin-2-yl)amino]phenyl}acetamide; 2-pyrrolidin-1-yl-N-{4-[(5-thien-3-ylpyrimidin-2-yl)amino]phenyl}acetamide; 4-pyrrolidin-1-yl-N-{4-[(5-thien-3-ylpyrimidin-2-yl)amino]phenyl}piperidine-1-carboxamide; N-(2-morpholin-4-ylethyl)-N′-{4-[(5-thien-3-ylpyrimidin-2-yl)amino]phenyl}urea; N-[2-(diethylamino)ethyl]-N′-{4-[(5-thien-3-ylpyrimidin-2-yl)amino]phenyl}urea; 3-(pyrrolidin-1-ylmethyl)-N-{4-[(5-thien-3-ylpyrimidin-2-yl)amino]phenyl}pyrrolidine-1-carboxamide; N-{4-[(5-thien-3-ylpyrimidin-2-yl)amino]phenyl}morpholine-4-carboxamide; 4-methyl-N-{4-[(5-thien-3-ylpyrimidin-2-yl)amino]phenyl}piperazine-1-carboxamide; N-{3-[(5-thien-3-ylpyrimidin-2-yl)amino]phenyl}acetamide; N-isopropyl-N′-(5-thien-3-ylpyrimidin-2-yl)benzene-1,3-diamine; N,N-diethyl-N′-(5-thien-3-ylpyrimidin-2-yl)benzene-1,3-diamine; N-(3-morpholin-4-ylphenyl)-5-thien-3-ylpyrimidin-2-amine; 4-(4-methyl-piperazin-1-ylmethyl)-N-[2-methyl-5-(5-thiophen-3-yl-pyrimidin-2-ylamino)-phenyl]-benzamide; N-(3-(5-(thiophen-3-yl)pyrazin-2-ylamino)phenyl)-2-(diethylamino)acetamide; 2-morpholin-4-yl-N-{3-[(5-thien-3-ylpyrazin-2-yl)amino]phenyl}acetamide; N-{3-[(5-thien-3-ylpyrazin-2-yl)amino]phenyl}morpholine-4-carboxamide; N-(2-morpholin-4-ylethyl)-N′-{3-[(5-thien-3-ylpyrazin-2-yl)amino]phenyl}urea; 4-pyrrolidin-1-yl-N-{3-[(5-thien-3-ylpyrazin-2-yl)amino]phenyl}piperidine-1-carboxamide; N-(3-(5-(thiophen-3-yl)pyrimidin-2-yl amino)phenyl)-2-(3-(pyrrolidin-1-yl)methyl)pyrrolidin-1-yl)acetamide; 4-amino-N-{4-[(5-thien-2-ylpyrimidin-2-yl)amino]phenyl}benzamide; 3-pyrrolidin-1-ylmethyl-pyrrolidine-1-carboxylic acid [3-(5-thien-3-ylpyrimidin-2-yl)amino)-phenyl]-amide; 1-(2-morpholin-4-yl-ethyl)-3-[3-(5-thien-3-ylpyrimidin-2-yl)amino)-phenyl]-urea; 1-(2-diethylamino-ethyl)-3-[3-(5-thien-3-ylpyrimidin-2-yl)amino)-phenyl]-urea; 1-(2-hydroxy-3-morpholin-4-yl-propyl)-3-[3-(5-thiophen-3-yl-pyrimidin-2-ylamino)-phenyl]-urea; 1-[2-(1,1-dioxo-1λ⁶-thiomorpholin-4-yl)ethyl]-3-[3-(5-thiophen-3-yl-pyrimidin-2-ylamino)-phenyl]-urea; 1-[2-(4-methyl-piperazin-1-yl)-ethyl]-3-[3-(5-thiophen-3-yl-pyrimidin-2-ylamino)-phenyl]-urea; 4-pyrrolidin-1-yl-piperidine-1-carboxylic acid [3-(5-thiophen-3-yl-pyrimidin-2-ylamino)-phenyl]-amide; 2-(4-pyrrolidin-1-yl-piperidine-1-yl)-N-[3-(5-thiophen-3-yl-pyrimidin-2-ylamino)-phenyl]-acetamide; 2-(2-morpholin-4-yl-ethylamino)-N-[3-(5-thiophen-3-yl-pyrimidin-2-ylamino)-phenyl]-acetamide; 2-(2-diethylamino-ethylamino)-N-[3-(5-thiophen-3-yl-pyrimidin-2-ylamino)-phenyl]-acetamide; 2-[2-(4-methyl-piperazin-1-yl)-ethylamino]-N-[3-(5-thiophen-3-yl-pyrimidin-2-ylamino)-phenyl]-acetamide; 2-(2-hydroxy-3-morpholin-4-yl-propylamino)-N-[3-(5-thiophen-3-yl-pyrimidin-2-ylamino)-phenyl]-acetamide; 2-[2-(1,1-dioxo-1λ⁶-thiomorpholin-4-yl)-ethylamino]-N-[3-(5-thiophen-3-yl-pyrimidin-2-ylamino)-phenyl]-acetamide; N-[4-(5-thiophen-2-yl-pyrimidin-2-ylamino)-phenyl]-acetamide; 2-morpholin-4-yl-N-[3-(5-thiophen-3-yl-pyrimidin-2-ylamino)-phenyl]-acetamide; 2-pyrrolidin-1-yl-N-[3-(5-thiophen-3-yl-pyrimidin-2-ylamino)-phenyl]-acetamide; 5-thiophen-2-yl-pyrimidin-2-ylamine; and 5-thiophen-3-yl-pyrimidin-2-ylamine, or a pharmaceutically acceptable salt, solvate or hydate thereof.

14. A pharmaceutical composition comprising the compound of claim 1 and a pharmaceutically acceptable carrier.

15. (canceled)

16. A method for treating FLK-1 or PDGFR mediated disorder in a mammal, the method comprising administering to the mammal a therapeutically effective amount of the composition of claim 14.

17. The method of claim 16, wherein the disorder is a cancer selected from the group consisting of squamous cell carcinoma, astrocytoma, Kaposi's sarcoma, glioblastoma, lung cancer, bladder cancer, head and neck cancer, melanoma, ovarian cancer, prostate cancer, breast cancer, small-cell lung cancer, glioma, colorectal cancer, genitourinary cancer and gastrointestinal cancer.

18. The method of claim 16, wherein the disorder is selected from the group consisting of diabetes, an autoimmune disorder, a hyperproliferation disorder, restenosis, fibrosis, psoriasis, von Heppel-Lindau disease, osteoarthritis, rheumatoid arthritis, angiogenesis, an inflammatory disorder, an immunological disorder and a cardiovascular disorder.
